(19) United States
(12) Patent Application Publication
(10) Pub. No.: US 2016/0258855 A1
(43) Pub. Date: Sep. 8, 2016

(54) Methods and Apparatus to Determine Diffusion Properties of Porous Structures for Drug Delivery

(71) Applicant: ForSight Vision4, Inc., Menlo Park, CA (US)

(72) Inventors: Kathleen Cogan Farinas, Menlo Park, CA (US); Cary Reich, Menlo Park, CA (US); Randolph E. Campbell, Menlo Park, CA (US); Signe Erickson, Menlo Park, CA (US); Michael S. Barrett, Menlo Park, CA (US)

(21) Appl. No.: 15/060,467

(22) Filed: Mar. 3, 2016

Related U.S. Application Data
(63) Continuation of application No. 13/884,343, filed on Oct. 14, 2013, now abandoned.
(60) Provisional application No. 61/412,642, filed on Nov. 11, 2010.

(51) Int. Cl. G01N 15/08; A61F 9/00; G01N 13/00; A61M 31/00

(52) U.S. Cl. CPC G01N 15/082; A61M 31/002; A61F 9/0017; G01N 13/00; G01N 2013/003

(57) ABSTRACT

Disclosed herein are improved therapeutic devices and methods and improved porous structures and measurement apparatus for use with therapeutic devices. In many embodiments, a porous structure is measured based on diffusion of the gas through the porous structure. The gas measurement may comprise an amount of gas measured to determine a resistance of the porous structure to diffusion. The diffusion of the gas through the porous structure can be used to determine release of a therapeutic agent through the porous structure, such that targeted amounts of therapeutic agent can be released for extended times and such that therapeutic device reservoir volume and porous frit structure can be tuned to release the therapeutic agent for an extended time above a target amount for the extended time.

### CROSS-REFERENCES TO RELATED APPLICATIONS

The present PCT application claims priority to U.S. Pat. App. Ser. No. 61/412,642 filed Nov. 11, 2010, entitled “Methods and Apparatus to Determine Porous Structures for Drug Delivery”, the full disclosure of which is incorporated herein by reference.

### STATEMENT AS TO RIGHTS TO INVENTIONS MADE UNDER FEDERALLY SPONSORED RESEARCH AND DEVELOPMENT

NOT APPLICAPLE

### BACKGROUND

This disclosure relates to the measurement and identification of porous structures for the release of therapeutic agents.

At least some of the prior methods and apparatus to determine the release rate of drugs from porous structures can be less than ideal in at least some instances. Although manufacturing processes can be controlled to provide porous structures, in at least some instances there can be at least some variability in the diffusion properties among manufactured porous structures. Although gas flow rates can be used to characterize at least some porous structures, in at least some instances at least some of the gas flow measurements can be less than ideal to determine diffusion properties of porous structures in at least some instances.

Work in relation to embodiments as described herein suggests that gas flow can be affected by the shape and size of channels and material used to form porous structures in at least some instances, and it may be helpful to have improved methods and apparatus to determine diffusion characteristics of porous structures before such structures are placed in the patient, for example before placed in the eye of the patient.

In light of the above, it would be desirable to provide improved methods and apparatus to determine properties of porous structures for therapeutic devices that overcome at least some of the above deficiencies.

### SUMMARY

Implementations described herein provide improved therapeutic devices and methods and improved porous structures and measurement apparatus to identify porous structures for use with therapeutic devices. In many implementations, a porous structure is measured based on diffusion of the fluid through the porous structure. The fluid may comprise one or more of a compressible fluid such as a gas or an incompressible fluid such as a liquid. The fluid measurement may comprise an amount of fluid measured to determine a resistance of the porous structure to diffusion, and the diffusion of the fluid through the porous structure may be measured when flow through the porous structure is inhibited. The diffusion of the fluid through the porous structure can be used to determine release of a therapeutic agent through the porous structure, such that targeted amounts of therapeutic agent can be released for extended times and such that therapeutic device reservoir volume and porous frit structure can be tuned to release the therapeutic agent for an extended time above a target amount. Alternatively or in combination, a resistance to gas flow through the porous structure can be measured, and one or more of a material or a channel structure of the porous structure identified, and the porous structure can be provided for use with a therapeutic device based the resistance to gas flow and the one or more of the material or the channel structure of the porous structure. In many implementationss, a container such as a chamber is sized to receive an assembled therapeutic device, and one or more of diffusion or gas flow through the porous structure is measured to determine that the therapeutic device is tuned to release the therapeutic amounts of the therapeutic agent for the extended time.

In a first aspect, described herein are implementations of a method of measuring diffusion of a fluid through a porous structure.

In many implementations, the porous structure is identified for use with a therapeutic device based on the diffusion.

In many implementations, flow of the fluid through the porous structure is inhibited to determine the diffusion.

In many implementations, the porous structure is placed at least partially in a housing of a therapeutic device wherein the diffusion of the first gas through the porous structure is measured.

In many implementations, a release rate of a therapeutic agent through the porous structure is determined based on the diffusion of the fluid through the porous structure.

The fluid may comprise one or more of a compressible fluid, a gas, a substantially incompressible fluid, a liquid, a solution, a solution comprising a solute, a solution comprising a small molecule, an aqueous solution comprising a small molecule, or an aqueous solution comprising a low molecular weight ion, or an aqueous solution comprising hydrogen ions, an acidic aqueous solution, or an alkali aqueous solution. In many implementations, the fluid may comprise the gas, and the gas comprises one or more of an elemental gas, helium gas, helium gas, nitrogen gas, oxygen gas, a noble gas, neon gas, argon gas, xenon gas, krypton gas, a compound gas molecule comprising a plurality of elements, carbon dioxide, nitrous oxide, a mixture of gas, or air.

In many implementations, the porous structure is coupled to the fluid on a first side of the porous structure and a second fluid on a second side of the structure, and the diffusion is determined by measuring one or more of, an amount of the fluid on the second side of the porous structure, an amount of the fluid on the first side of the porous structure, an amount of the second fluid on the first side of the porous structure, or an amount of the second fluid on the second side of the porous structure.

In many implementations, the fluid comprises a first gas and the second fluid comprises a second gas.

In many implementations, the first gas is contained in a first chamber and has a first amount of pressure and the second gas is contained in a second chamber and has a second amount of pressure and wherein the first amount of pressure is substantially similar to the second amount of pressure such that flow of the first gas and the second gas through the porous structure is substantially inhibited.

In many implementations, the first gas is measured at a first time and a second time to determine a resistance to diffusion of the porous structure.

In a related aspect, implementations provide an apparatus to determine diffusion. A support is configured to receive a porous structure. The apparatus comprises a first source of a first fluid, and a second source of a second fluid. A container comprises the first fluid, and a detector is configured to measure one or more of the first fluid or the second fluid in response to diffusion of the first fluid through the porous structure opposite the second fluid.

In many implementations, a valve is configured to couple the container to the second source of fluid when the container comprises the first fluid.

In many implementations, circuitry, such as a processor or array logic is coupled to the valve and the detector. The processor comprises a computer readable memory having instructions of a computer program embodied thereon to open the valve to couple the container to the second fluid and measure an amount of the one or more of the first fluid or the second fluid in response to the open valve.

In many implementations, the processor instructions are configured to open the valve and measure the amount when the valve has been opened an amount of time of at least about one tenth of a second.

In many implementations, the first fluid comprises a first gas and the second fluid comprises a second gas and wherein the processor has instructions to open a first gas valve coupled to a first source of a first gas to provide gas to the chamber and wherein the instructions are configured to open the valve to couple the second fluid to the container when the first valve is closed.

In many implementations, the processor instructions are configured to provide a time delay between closing a gas valve coupled to the first source of the first fluid and opening the valve that couples the second fluid to the container.

In many implementations, further a second container is coupled to a second source of the second fluid and wherein the valve couples the first container to the second container when opened.

In many implementations, circuitry is coupled to the valve and the detector. The circuitry comprising one or more of a processor or logic circuitry configured to open the valve to accumulate the first fluid in the second container and measure the amount when the first fluid has accumulated in the second chamber and the second fluid has accumulated in the first chamber. The circuitry may comprise logic circuitry, such as programmable array logic circuitry (hereinafter “PAL” circuitry). Alternatively or in combination, the circuitry may comprise the processor. The processor may comprise a computer readable memory having instructions of a computer program embodied thereon to open the valve to accumulate the first fluid in the second container and measure the amount when the first fluid has accumulated in the second chamber and the second fluid has accumulated in the first chamber.

In many implementations, a second valve is configured to couple the second chamber to the detector and wherein the instructions are configured to open the second valve to couple the detector to the second chamber when the first fluid has accumulated in the second chamber. The processor instructions can be configured to open the second valve when the valve is closed so as to inhibit release of the first gas from the first chamber when the second valve is open. Alternatively or in combination, the logic circuitry, such as the PAL circuitry can be configured to open the second valve when the valve is closed so as to inhibit release of the first gas from the first chamber when the second valve is open.

In many implementations, the detector is configured to measure the first gas and wherein the processor instructions are configured to measure an amount of the first gas accumulated in the second chamber.

In many implementations, a pressure coupling device is configured to inhibit flow of the first fluid and the second fluid through the porous structure, the pressure coupling device configured to couple a first pressure of the first container to a second pressure of the second container such that the first pressure corresponds substantially to the second pressure and wherein the pressure coupling device comprises one or more of a diaphragm coupled between the first container or the second container, a pressure equalization column, or atmospheric pressure coupled to the first container and the second container.

In many implementations, one or more of a first pressure sensor is configured to measure a first pressure of the first container or a second pressure sensor to measure a second pressure of the second container.

In many implementations, further comprising one or more of a first temperature sensor to measure a first pressure of the first container or a second temperature sensor to measure a second pressure of the second container.

In many implementations, the support comprises a lower surface of the container.

In many implementations, the support comprises an opening sized to receive the first porous structure.

In many implementations, the support comprises a mount and the mount is sized to receive a housing of a therapeutic device with the porous structure mounted on the therapeutic device for release of a therapeutic agent into an eye and wherein resistance to diffusion of the gas through the porous structure is determined. The mount can be sized and may comprise a material having a thickness so as to inhibit penetration of the first fluid from the container or the second fluid into the container.

In many implementations, the container is sized to receive an assembled therapeutic device having a device chamber and the support is configured to hold the therapeutic device in the container when the container is sealed.

In many implementations, container comprises a plurality of sealable chambers, each chamber sized to hold the therapeutic device when sealed and wherein instructions of a processor are configured to measure one or more of the first gas or the second gas.

In a related aspect, implementations provide a method measuring an assembled therapeutic device. The assembled therapeutic device is placed in a first container, the first container comprising a first fluid, wherein the assembled therapeutic device comprises a device chamber to store a therapeutic agent and the first fluid accumulates in the device chamber. A valve is opened to couple the first container to a second fluid, and an amount of one or more of the first fluid or the second fluid is measured.

In many implementations, a therapeutic agent has a half-life within the device chamber corresponding to a half-life of the first fluid in the device chamber.

In many implementations, the device chamber comprises a substantially constant volume.

In many implementations, the first fluid comprises a first gas and the second fluid comprises a second gas and wherein the first container comprises a first chamber having the assembled drug delivery device placed therein.

In many implementations, the first gas as is accumulated in a second container when the valve is open and wherein the second gas is measured.

In many implementations, the valve is closed and a second valve is opened to couple the second chamber to a detector with a channel extending between the detector and the second chamber and wherein the first gas accumulated in the second chamber is measured. The second valve can be opened when the valve is closed so as to inhibit release of the first gas from the chamber when the second valve is open.

In a related aspect, implementations provide a method. A plurality of assembled therapeutic devices is placed in a plurality of first chambers, the plurality of first chambers comprising a first gas, wherein each of the plurality of assembled therapeutic devices comprises a porous structure and a device chamber to store a therapeutic agent and wherein the first gas accumulates within said each device chamber. A plurality of first valves is opened to couple the plurality of first chambers to a plurality of second chambers comprising a second gas. A second plurality of second valves is opened to couple the plurality of second chambers to a detector. An amount of one or more of the first gas or the second gas is measured with the detector to determine diffusion of the porous structure of said each of the plurality of assembled therapeutic devices.

In another related aspect, implementations provide an apparatus. The apparatus comprises first source of a first gas, and a first plurality of chambers sized to receive a plurality of assembled therapeutic devices, the plurality of chambers coupled to the source of the first gas. A second plurality of chambers coupled to a second source of a second gas. A first plurality valves to couple the first plurality of chamber to the second plurality of chambers. A detector to measure the first gas or the second gas, and a second plurality of valves coupled to the detector and the second plurality of chambers to measure an amount of the first gas or the second gas for each of the second plurality of chambers.

In many implementations, further comprising a processor coupled to the first plurality of valves and the second plurality of valves, the processor comprising a computer readable memory having instructions of the computer program stored thereon, the instructions configured to open the first plurality of valves to couple the first plurality of chambers to the second plurality of chambers when the first plurality of chambers comprises the first gas and the second plurality of chambers comprises the second gas, the instructions configured to open the second plurality of valves to couple the plurality of second chambers to the detector to measure the amount of the first gas or the second gas for each of the second plurality of chambers.

In many implementations, the processor comprises instructions to open and close each of the second plurality of valves sequentially to couple the detector sequentially to each of the plurality of second chambers.

In many implementations, a plurality of channels extends from the detector to the second plurality of valves to couple the detector to the second plurality of chambers.

In another related aspect, implementations provide a method of measuring an assembled therapeutic device. The assembled therapeutic device in a first container, the first container comprising a first solution comprising a first solute, wherein the assembled therapeutic device comprises a device chamber to store a therapeutic agent and the first solution accumulates in the device chamber. A valve is opened to couple the first container to a second container, the second container comprising a second solution comprising a second solute. One or more of the first solute or the second solute is measured.

In another related aspect, implementations provide a method. A first resistance to flow of a first fluid through a porous structure is measured. A second resistance to flow of a second fluid through porous structure is measured. The porous structure is provided for use with a therapeutic device based the first flow and the second flow. The porous structure may be identified for use based on the first flow and the second flow.

In many implementations, the first flow and the second flow correspond to release of the therapeutic agent from the device.

In many implementations, the first flow and the second flow correspond to a volume of a chamber of the therapeutic device to release the therapeutic agent for an extended time.

In many implementations, the first fluid comprises a first viscosity and the second fluid comprises a second viscosity different from the first viscosity.

In many implementations, the first fluid comprises a gas and the second fluid comprises a gas.

In many implementations, the first fluid comprises a liquid and the second fluid comprises a gas.

In another related aspect, implementations provide a method. A resistance to gas flow through a porous structure is measured. One or more of a material or a channel structure of the porous structure is identified. The porous structure is provided for use with a therapeutic device based the resistance to gas flow and the one or more of the material or the channel structure of the porous structure.

In many implementations, the therapeutic device comprises a device chamber volume sized to receive a therapeutic agent and wherein the resistance to gas flow and the one or more of the material or the channel structure correspond volume of the device chamber.

In many implementations, the therapeutic device is at least partially assembled when the resistance to flow is measured such that the gas flows through the chamber and the porous structure.

In another related aspect, implementations provide a method. A therapeutic device is provided, the therapeutic device comprising a device chamber, a penetrable barrier and a porous structure. The therapeutic device is placed in a chamber. A resistance to gas flow through the porous structure is measured when the therapeutic device is placed in the chamber.

In many implementations, the chamber comprises a first pressure and the device chamber comprises a second pressure such that gas flows through the porous structure when the chamber is defined with the penetrable barrier, a housing of the therapeutic device, and the porous structure.

In many implementations, the volume of the device chamber remains substantially constant when the therapeutic device is placed in the chamber and the resistance to gas flow is measured.

In many implementations, the housing and the porous structure each comprise a rigid material such that a volume of the device chamber remains substantially constant.

In many implementations, a valve is opened to couple the chamber, a second chamber with a channel extending from the first chamber to the second chamber and wherein the gas accumulates in the device chamber or the second chamber when the valve is open.

In another related aspect, implementations provide an apparatus. The apparatus comprises a first chamber sized to receive a therapeutic device comprising a device chamber, a penetrable barrier, and a porous structure. A second chamber is coupled to the first chamber, and a channel extends between the first chamber and the second chamber. A valve is located along the channel to couple the first chamber to the second chamber when the valve is open and isolate the first chamber from the second chamber when the valve is closed. A source of gas provides a concentration gradient between the first chamber and the second chamber when the valve is closed. A gas sensor is coupled to one or more of the first chamber or the second chamber to determine diffusion of the gas across the porous structure in response to the concentration gradient when the valve has opened.

In many implementations, the comprises circuitry coupled to the pressure sensor to indentify a tuned response of the device chamber and the porous structure corresponding to a tuned relase of a formulation of a therapeutic agent placed in the device chamber.

### BRIEF DESCRIPTION OF THE DRAWINGS

FIG. 1 shows an eye suitable for incorporation of the therapeutic device in accordance with an implementation;

FIG. 1A-1 shows a therapeutic device implanted at least partially within the eye as in FIG. 1, in accordance with an implementation;

FIG. 2 shows a therapeutic device implanted under the conjunctiva and extending through the sclera to release a therapeutic agent into vitreous humor of the eye so as to treat the retina of the, in accordance with an implementation;

FIG. 3 shows structures of a therapeutic device configured for placement in an eye, in accordance with an implementation;

FIG. 4 shows therapeutic device loaded into an insertion cannula of an insertion apparatus, in accordance with an implementation;

FIG. 5 shows a therapeutic device comprising a reservoir suitable for loading in a cannula, in accordance with an implementation;

FIG. 6A-1 shows a therapeutic device comprising a container having a penetratable barrier disposed on a first end, a porous structure disposed on a second end to release therapeutic agent for an extended period, and a retention structure comprising an extension protruding outward from the container to couple to the sclera and the conjunctiva, in accordance with an implementation;

FIG. 6A-2 shows a therapeutic device as in FIG. 6A-1 comprising a rounded distal end, in accordance with an implementation;

FIG. 6B shows a rigid porous structure configured for sustained release with a device as in FIG. 6A-1, in accordance with an implementation;

FIG. 6B-1 shows interconnecting channels extending from a first side to a second side of the porous structure as in FIG. 6B;

FIG. 6B-2 shows a plurality of paths of the therapeutic agent along the interconnecting channels extending from a first side to a second side of the porous structure as in FIGS. 6B and 6B1;

FIG. 6B-3 shows blockage of the openings with a covering and the plurality of paths of the therapeutic agent along the interconnecting channels extending from a first side to a second side of the porous structure as in FIGS. 6B and 6B-1;

FIG. 6B-4 shows blockage of the openings with particles and the plurality of paths of the therapeutic agent along the interconnecting channels extending from a first side to a second side of the porous structure as in FIGS. 6B and 6B-1;

FIG. 6B-5 shows an effective cross-sectional size and area corresponding to the plurality of paths of the therapeutic agent along the interconnecting channels extending from a first side to a second side of the porous structure as in FIGS. 6B and 6B-1;

FIG. 6C shows a rigid porous structure as in FIG. 6B incorporated into a sclera tack, in accordance with an implementation;

FIG. 6D, shows a rigid porous structure as in FIG. 6B coupled with a reservoir for sustained release, in accordance with an implementation;

FIG. 6E shows a rigid porous structure as in FIG. 6B comprising a hollow body or tube for sustained release, in accordance with an implementation;

FIG. 6F shows a rigid porous structure as in FIG. 6B comprising a non-linear helical structure for sustained release, in accordance with an implementation;

FIG. 6G shows porous nanostructures, in accordance with an implementation;

FIG. 7 shows a therapeutic device coupled to an injector that removes material from the device and injects therapeutic agent into the device, in accordance with an implementation;

FIG. 7A shows a therapeutic device comprising a porous structure and a penetrable barrier as in FIG. 6A-1, with the penetrable barrier coupled to an injector to inject and remove material from the device, in accordance with an implementation;

FIG. 7A-1 shows a therapeutic device coupled to an injector needle comprising a stop that positions the distal end of the needle near the proximal end of the device to flush the reservoir with ejection of liquid formulation through the porous frit structure, in accordance with an implementation;

FIG. 7A-2 shows a therapeutic device comprising a penetrable barrier coupled to an injector to inject and remove material from the device such that the liquid in the reservoir is exchanged with the injected formulation, in accordance with an implementation;

FIG. 7B-1 shows a side cross-sectional view of a therapeutic device comprising a retention structure having a cross-section sized to fit in an elongate incision, in accordance with an implementation;

FIG. 7B-2 shows an isometric view of the therapeutic device as in FIG. 7B-1;

FIG. 7B-3 shows a top view of the therapeutic device as in FIG. 7B-1;

FIG. 7B-4 shows a side cross sectional view along the short side of the retention structure of the therapeutic device as in FIG. 7B-1;

FIG. 7B-5 shows a bottom view of the therapeutic device as in FIG. 7B-1 implanted in the sclera;

FIG. 7B-5A shows a cutting tool comprising a blade having a width corresponding to the perimeter of the barrier and the perimeter of the narrow retention structure portion, in accordance with an implementation;

FIGS. 7B-6A and 7B-6B show distal cross-sectional view and a proximal cross-sectional view, respectively, of a therapeutic device comprising an elongate and non-circular cross-sectional size, in accordance with an implementation;

FIG. 7B-6C shows an isometric view of the therapeutic device having a retention structure with an elongate cross-sectional size, in accordance with an implementation;

FIG. 7B-6D shows a distal end view of the therapeutic device as in FIG. 7B-6C;

FIG. 7B-6E1 shows a side view of the short axis of the narrow neck portion of the therapeutic device as in FIG. 7B-6C;

FIG. 7B-6E2 shows a side view of the long axis of the narrow neck portion of the therapeutic device as in FIG. 7B-6C;

FIG. 7B-6F shows a proximal view of the therapeutic device as in FIGS. 7B-6C;

FIG. 7B-6G to FIG. 7B-6I show exploded assembly drawings for the therapeutic device as in FIGS. 7B-6C to 7B-6F;

FIGS. 8A and 8B show scanning electron microscope images from fractured edges of porous frit structures so as to show the structure of the porous structure to release the therapeutic agent, in accordance with implementations of the present invention;

FIGS. 9A and 9B show scanning electron microscope images from surfaces of porous frit structures, in accordance with an implementation;

FIG. 10 shows a pressure decay test and test apparatus for use with a porous structure so as to identify porous frit structures suitable for use with therapeutic devices in accordance with an implementation;

FIG. 11 shows a pressure flow test and test apparatus suitable for use with a porous structure so as to identify porous frit structures suitable for use with therapeutic devices in accordance with an implementation;

FIGS. 12A and 12A1 show a side cross sectional view and a top view, respectively, of a therapeutic device for placement substantially between the conjunctiva and the sclera, in accordance with an implementation;

FIG. 12A2 shows the therapeutic device implanted with the reservoir between the conjunctiva and the sclera, such that elongate structure extends through the sclera to couple the reservoir chamber to the vitreous humor, in accordance with an implementation;

FIG. 12B shows the porous structure of therapeutic device located in channel near the opening to the chamber of the container, in accordance with an implementation;

FIG. 12C shows the porous structure located within the chamber of container and coupled to the first opening of the elongate structure so as to provide the release rate profile, in accordance with an implementation;

FIG. 12D shows a plurality of injection ports spaced apart so as to inject and exchange the liquid of chamber, in accordance with an implementation;

FIG. 13 shows the elongate structure coupled to the container away from the center of container and near and located near an end of the container, in accordance with an implementation;

FIG. 14A shows a porous frit structure composed of sintered metal powder, in accordance with an implementation;

FIG. 14B shows a porous frit structure having sintered metal fibers, in accordance with an implementation;

FIG. 14C show a scanning electron micrograph (hereinafter “SEM”) of a porous frit structure comprising sintered Ti, in accordance with an implementation;

FIG. 15 shows an apparatus to determine a release rate of a therapeutic agent through a porous structure based on gas diffusion, in accordance with an implementation;

FIG. 16A shows a test apparatus configured to measure diffusion of a fluid through a porous structure, in accordance with an implementation;

FIG. 16A1 shows a test apparatus configured to measure diffusion of a gas through a porous structure in which the porous structure is coupled to a housing of the therapeutic device when the housing is mounted in the test apparatus, in accordance with an implementation;

FIG. 16B shows the assembled therapeutic device placed in the first container, for example first chamber, in accordance with an implementation;

FIG. 16C shows a plurality of assembled therapeutic devices placed in a plurality of containers, for example a plurality of chambers, in accordance with an implementation;

FIG. 17 shows a method of identifying a porous structure of a therapeutic device in accordance with an implementation; and

FIGS. 18A to 18C show a comparison of flow rate data and RRI's for sintered titanium and sintered stainless steel, in accordance with an implementation; and

FIG. 19 shows stability data for a formulation of Lucentis that can be used to identify materials for porous frit structures, in accordance with an implementation.

### DETAILED DESCRIPTION

Embodiments described herein can be used in many ways to characterize porous structure, and can be well suited to provide improved porous structures for the release of therapeutic agents with implantable devices. The porous structures measured and identified for use with therpapeutic devices as described herein can be used to deliver one or more of many therapeutic agents. Although specific reference is made to sintered porous structures for the delivery of macromolecules comprising antibodies or antibody fragments to the posterior segment of the eye, embodiments described herein can be used to identify porous structures for many devices where diffusion through the porous structure can be helpful, such as to deliver one or more of many therapeutic agents to many tissues of the body. For example, embodiments described herein can be used to identify porous structures for the delivery of a therapeutic agent to one or more of the following tissues: intravascular, intra-articular, intrathecal, pericardial, intraluminal and gut.

Examples of porous structures that can be measured with the methods and apparatus as described herein are described in U.S. application Ser. No. 12/696,678, filed 29 Jan. 2010, entitled “Posterior Segment Drug Delivery”, Published as US Pub. No. 2010/0255061 on Oct. 7, 2010 the full disclosure of which is incorporated herein by reference and suitable for combination in accordance with embodiments as described herein.

As used herein, like alpha-numeric references are used to describe like structures, including like structures of the aforementioned U.S. Patent Publication No. 20100255061, the full disclosure of which has been previously incorporated by reference.

As used herein the release rate index encompasses (PA/FL) where P comprises the porosity, A comprises an effective area, F comprises a curve fit parameter corresponding to an effective length and L comprises a length or thickness of the porous structure. The units of the release rate index (RRI) comprise units of mm unless indicated otherwise and can be determine by a person of ordinary skill in the art in accordance with the teachings described hereon.

As used herein, sustained release encompasses release of therapeutic amounts of an active ingredient of a therapeutic agent for an extended period of time. The sustained release may encompass first order release of the active ingredient, zero order release of the active ingredient, or other kinetics of release such as intermediate to zero order and first order, or combinations thereof.

As used herein a therapeutic agent referred to with a trade name encompasses one or more of the formulation of the therapeutic agent commercially available under the tradename, the active ingredient of the commercially available formulation, the generic name of the active ingredient, or the molecule comprising the active ingredient.

As used herein, similar numerals indicate similar structures and/or similar steps.

The therapeutic agent may be contained within a chamber of a container, for example within a reservoir comprising the container and chamber. The therapeutic agent may comprise a formulation such as solution of therapeutic agent, a suspension of a therapeutic agent or a dispersion of a therapeutic agent, for example. Examples of therapeutic agents suitable for use in accordance with embodiments of the therapeutic device are described herein, for example with reference to Table 1A below and elsewhere.

The therapeutic agent may comprise a macromolecule, for example an antibody or antibody fragment. The therapeutic macromolecule may comprise a VEGF inhibitor, for example commercially available Lucentis™. The VEGF (Vascular Endothelial Growth Factor) inhibitor can cause regression of the abnormal blood vessels and improvement of vision when released into the vitreous humor of the eye. Examples of VEGF inhibitors include Lucentis™, Avastin™, Macugen™, and VEGF Trap.

The therapeutic agent may comprise small molecules such as of a corticosteroid and analogues thereof. For example, the therapeutic corticosteroid may comprise one or more of triamcinolone, triamcinolone acetonide, dexamethasone, dexamethasone acetate, fluocinolone, fluocinolone acetate, or analogues thereof. Alternatively or in combination, the small molecules of therapeutic agent may comprise a tyrosine kinase inhibitor comprising one or more of axitinib, bosutinib, cediranib, dasatinib, erlotinib, gefitinib, imatinib, lapatinib, lestaurtinib, nilotinib, semaxanib, sunitinib, toceranib, vandetanib, or vatalanib, for example.

The therapeutic agent may comprise an anti-VEGF therapeutic agent. Anti-VEGF therapies and agents can be used in the treatment of certain cancers and in age-related macular degeneration. Examples of anti-VEGF therapeutic agents suitable for use in accordance with the embodiments described herein include one or more of monoclonal antibodies such as bevacizumab (Avastin™) or antibody derivatives such as ranibizumab (Lucentis™), or small molecules that inhibit the tyrosine kinases stimulated by VEGF such as lapatinib (Tykerb™), sunitinib (Sutent™), sorafenib (Nexavar™), axitinib, or pazopanib.

The therapeutic agent may comprise a therapeutic agent suitable for treatment of dry age related macular degeneration (hereinafter “AMD”) such as one or more of Sirolimus™ (Rapamycin), Copaxone™ (Glatiramer Acetate), Othera™, Complement C5aR blocker, Ciliary Neurotrophic Factor, Fenretinide or Rheopheresis.

The therapeutic agent may comprise a therapeutic agent suitable for treatment of wet AMD such as one or more of REDD14NP (Quark), Sirolimus™ (Rapamycin), ATG003; Regeneron™ (VEGF Trap) or complement inhibitor (POT-4).

The therapeutic agent may comprise a kinase inhibitor such as one or more of bevacizumab (monoclonal antibody), BIBW 2992 (small molecule targeting EGFR/Erb2), cetuximab (monoclonal antibody), imatinib (small molecule), trastuzumab (monoclonal antibody), gefitinib (small molecule), ranibizumab (monoclonal antibody), pegaptanib (small molecule), sorafenib (small molecule), dasatinib (small molecule), sunitinib (small molecule), erlotinib (small molecule), nilotinib (small molecule), lapatinib (small molecule), panitumumab (monoclonal antibody), vandetanib (small molecule) or E7080 (targeting VEGFR2/VEGFR2, small molecule commercially available from Esai, Co.)

The amount of therapeutic agent within the therapeutic device may comprise from about 0.01 mg to about 10 mg, for example Lucentis™, so as to provide therapeutic amounts of the therapeutic agent for the extended time, for example at least 30 days. The extended time may comprise at least 90 days or more, for example at least 180 days or for example at least 1 year, at least 2 years or at least 3 years or more. The target threshold therapeutic concentration of a therapeutic agent such as Lucentis™ in the vitreous may comprise at least a therapeutic concentration of 0.1 ug/mL. For example, the target threshold concentration may comprise from about 0.1 ug/mL to about 5 ug/mL for the extended time, where the upper value is based upon calculations shown in Examples of U.S. Pat. App. Pub. No. 2010/0255061, entitled “Posterior Segment Drug Delivery, the full disclosure of which has been previsously incorporated herein by reference. The target threshold concentration is drug dependent and thus may vary for other therapeutic agents.

The delivery profile may be configured in many ways to obtain a therapeutic benefit from the sustained release device. For example, an amount of the therapeutic agent may be inserted into the container at monthly intervals so as to ensure that the concentration of therapeutic agent is above a safety protocol or an efficacy protocol for the therapeutic agent, for example with monthly or less frequent injections into the container. The sustained release can result in an improved delivery profile and may result in improved results. For example, the concentration of therapeutic agent may remain consistently above a threshold amount, for example 0.1 ug/mL, for the extended time.

The insertion method may comprise inserting a dose into the container of the therapeutic device. For example, a single injection of Lucentis™ may be injected into the therapeutic device.

The duration of sustained delivery of the therapeutic agent may extend for twelve weeks or more, for example four to six months from a single insertion of therapeutic agent into the device when the device is inserted into the eye of the patient.

The therapeutic agent may be delivered in many ways so as to provide a sustained release for the extended time. For example, the therapeutic device may comprise a therapeutic agent and a binding agent. The binding agent may comprise small particles configured to couple releasably or reversibly to the therapeutic agent, such that the therapeutic agent is released for the extended time after injection into the vitreous humor. The particles can be sized such that the particles remain in the vitreous humor of the eye for the extended time.

The therapeutic agent may be delivered with a device implanted in the eye. For example, the drug delivery device can be implanted at least partially within the sclera of the eye, so as to couple the drug delivery device to the sclera of the eye for the extended period of time. The therapeutic device may comprise a drug and a binding agent. The drug and binding agent can be configured to provide the sustained release for the extended time. A membrane or other diffusion barrier or mechanism may be a component of the therapeutic device to release the drug for the extended time.

The lifetime of the therapeutic device and number of injections can be optimized for patient treatment. For example, the device may remain in place for a lifetime of 30 years, for example with AMD patients from about 10 to 15 years. For example, the device may be configured for an implantation duration of at least two years, with 8 injections (once every three months) for sustained release of the therapeutic agent over the two year duration. The device may be configured for implantation of at least 10 years with 40 injections (once every three months) for sustained release of the therapeutic agent.

The therapeutic device can be refilled in many ways. For example, the therapeutic agent can be refilled into the device in the physician's office.

The therapeutic device may comprise many configurations and physical attributes, for example the physical characteristics of the therapeutic device may comprise at least one of a drug delivery device with a suture, positioning and sizing such that vision is not impaired, and biocompatible material. The device may comprise a reservoir capacity from about 0.005 cc to about 0.2 cc, for example from about 0.01 cc to about 0.1 cc, and a device volume of no more than about 2 cc. A vitrectomy may be performed for device volumes larger than 0.1 cc. The length of the device may not interfere with the patient's vision and can be dependent on the shape of the device, as well as the location of the implanted device with respect to the eye. The length of the device may also depend on the angle in which the device is inserted. For example, a length of the device may comprise from about 4 to 6 mm. Since the diameter of the eye is about 24 mm, a device extending no more than about 6 mm from the sclera into the vitreous may have a minimal effect on patient vision.

Embodiments may comprise many combinations of implanted drug delivery devices. The therapeutic device may comprise a drug and binding agent. The device may also comprise at least one of a membrane, an opening, a diffusion barrier, a diffusion mechanism so as to release therapeutic amounts of therapeutic agent for the extended time.

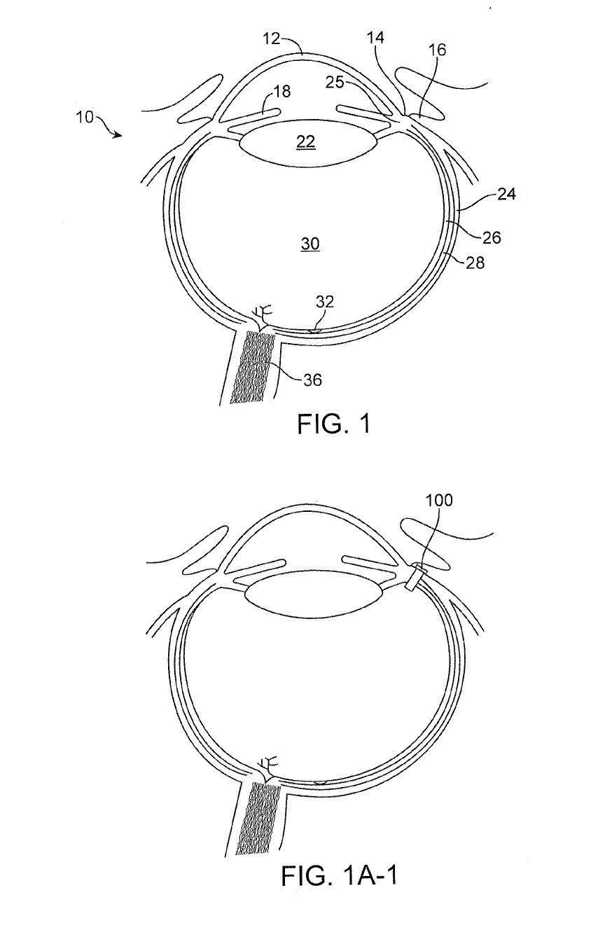

FIG. 1 shows an eye 10 suitable for incorporation of the therapeutic device. The eye has a cornea 12 and a lens 22 configured to form an image on the retina 26. The cornea can extend to a limbus 14 of the eye, and the limbus can connect to a sclera 24 of the eye. A conjunctiva 16 of the eye can be disposed over the sclera. The lens can accommodate to focus on an object seen by the patient. The eye has an iris 18 that may expand and contract in response to light. The eye also comprises a choroid 28 disposed between the sclera 24 and the retina 26. The retina comprises the macula 32. The eye comprises a pars plana 25, which comprises an example of a region of the eye suitable for placement and retention, for example anchoring, of the therapeutic device 100 as described herein. The pars plana region may comprise sclera and conjunctiva disposed between the retina and cornea. The therapeutic device can be positioned so as to extend from the pars plana region into the vitreous humor 30 to release the therapeutic agent. The therapeutic agent can be released into the vitreous humor 30, such that the therapeutic agent arrives at the retina and choroids for therapeutic effect on the macula. The vitreous humor of the eye comprises a liquid disposed between the lens and the retina. The vitreous humor may comprise convection currents to deliver the therapeutic agent to the macula.

FIG. 1A-1 shows a therapeutic device implanted at least partially within the eye as in FIG. 1. The therapeutic device can be implanted at least partially within the eye in many ways as described herein, for example.

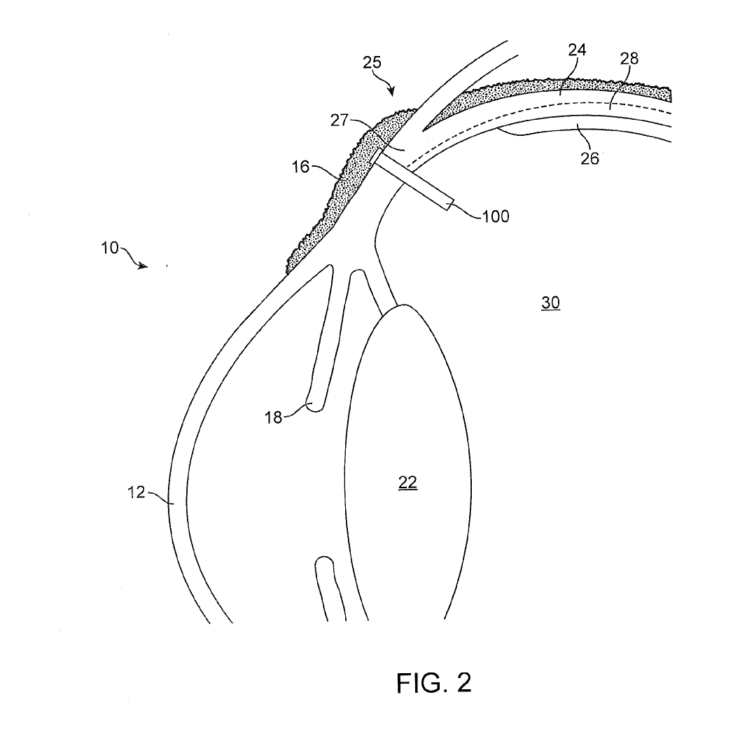

FIG. 2 shows a therapeutic device 100 implanted under the conjunctiva 16 and extending through the sclera 24 to release a therapeutic agent 110 into vitreous humor 30 of the eye 10 so as to treat the retina of the eye. The therapeutic device 100 may comprise a retention structure 120 such as a smooth protrusion configured for placement along the sclera and under the conjunctiva, such that the conjunctiva can cover the therapeutic device and protect the therapeutic device 100. When the therapeutic agent 110 is inserted into the device 100, the conjunctiva may be lifted away, incised, or punctured with a needle to access the therapeutic device. The eye may comprise an insertion of the tendon 27 of the superior rectus muscle to couple the sclera of the eye to the superior rectus muscle. The device 100 may be positioned in many locations of the pars plana region, for example away from tendon 27 and one or more of posterior to the tendon, posterior to the tendon, under the tendon, or with nasal or temporal placement of the therapeutic device.

While the implant can be positioned in the eye in many ways, work in relation to embodiments suggests that placement in the pars plana region can release therapeutic agent into the vitreous to treat the retina, for example therapeutic agent comprising an active ingredient composed of large molecules.

Therapeutic agents 110 suitable for use with device 100 includes many therapeutic agents, for example as listed in Table 1A, herein below. The therapeutic agent 110 of device 100 may comprise one or more of an active ingredient of the therapeutic agent, a formulation of the therapeutic agent, a commercially available formulation of the therapeutic agent, a physician prepared formulation of therapeutic agent, a pharmacist prepared formulation of the therapeutic agent, or a commercially available formulation of therapeutic agent having an excipient. The therapeutic agent may be referred to with generic name or a trade name, for example as shown in Table 1A.

The therapeutic device 100 can be implanted in the eye to treat the eye for as long as is helpful and beneficial to the patient. For example the device can be implanted for at least about 5 years, such as permanently for the life of the patient. Alternatively or in combination, the device can be removed when no longer helpful or beneficial for treatment of the patient.

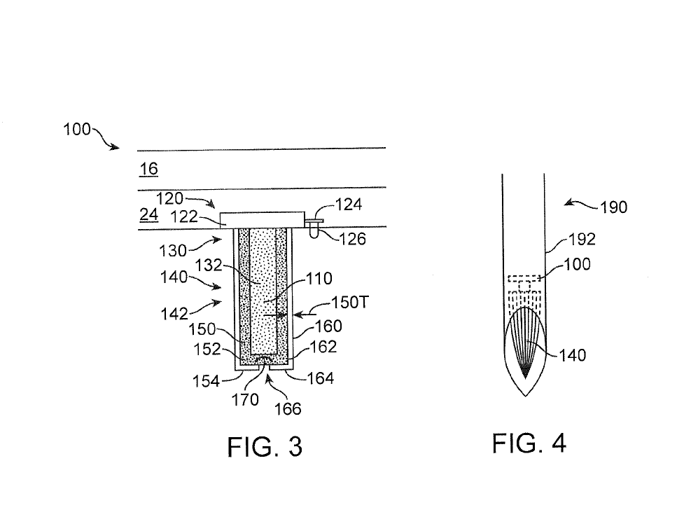

FIG. 3 shows structures of therapeutic device 100 configured for placement in an eye. The device may comprise retention structure 120 to couple the device 100 to the sclera, for example a protrusion disposed on a proximal end of the device. The device 100 may comprise a container 130 affixed to the retention structure 120. An active ingredient, for example therapeutic agent 110, can be contained within a reservoir 140, for example a chamber 132 defined by a container 130 of the device. The container 130 may comprise a porous structure 150 comprising a porous material 152, for example a porous glass frit 154, and a barrier 160 to inhibit release of the therapeutic agent, for example non-permeable membrane 162. The non-permeable membrane 162 may comprise a substantially non-permeable material 164. The non-permeable membrane 162 may comprise an opening 166 sized to release therapeutic amounts of the therapeutic agent 110 for the extended time. The porous structure 150 may comprise a thickness 150T and pore sizes configured in conjunction with the opening 166 so as to release therapeutic amounts of the therapeutic agent for the extended time. The container 130 may comprise reservoir 140 having a chamber with a volume 142 sized to contain a therapeutic quantity of the therapeutic agent 110 for release over the extended time. The device may comprise a needle stop 170. Proteins in the vitreous humor may enter the device and compete for adsorption sites on the porous structure and thereby may contribute to the release of therapeutic agent. The therapeutic agent 110 contained in the reservoir 140 can equilibrate with proteins in the vitreous humor, such that the system is driven towards equilibrium and the therapeutic agent 110 is released in therapeutic amounts.

The non-permeable membrane 162, the porous material 152, the reservoir 140, and the retention structure 120, may comprise many configurations to deliver the therapeutic agent 110. The non-permeable membrane 162 may comprise an annular tube joined by a disc having at least one opening formed thereon to release the therapeutic agent. The porous material 152 may comprise an annular porous glass frit 154 and a circular end disposed thereon. The reservoir 140 may be shape-changing for ease of insertion, i.e., it may assume a thin elongated shape during insertion through the sclera and then assume an extended, ballooned shape, once it is filled with therapeutic agent.

The porous structure 150 can be configured in many ways to release the therapeutic agent in accordance with an intended release profile. For example, the porous structure may comprise a porous structure having a plurality of openings on a first side facing the reservoir and a plurality of openings on a second side facing the vitreous humor, with a plurality of interconnecting channels disposed therebetween so as to couple the openings of the first side with the openings of the second side, for example a sintered rigid material. The porous structure 150 may comprise one or more of a permeable membrane, a semi-permeable membrane, a material having at least one hole disposed therein, nano-channels, nano-channels etched in a rigid material, laser etched nano-channels, a capillary channel, a plurality of capillary channels, one or more tortuous channels, tortuous microchannels, sintered nano-particles, an open cell foam or a hydrogel such as an open cell hydrogel.

FIG. 4 shows therapeutic device 100 loaded into an insertion cannula 192 of an insertion apparatus 190, in which the device 100 comprises an elongate narrow shape for insertion into the sclera, and in which the device is configured to expand to a second elongate wide shape for retention at least partially in the sclera;

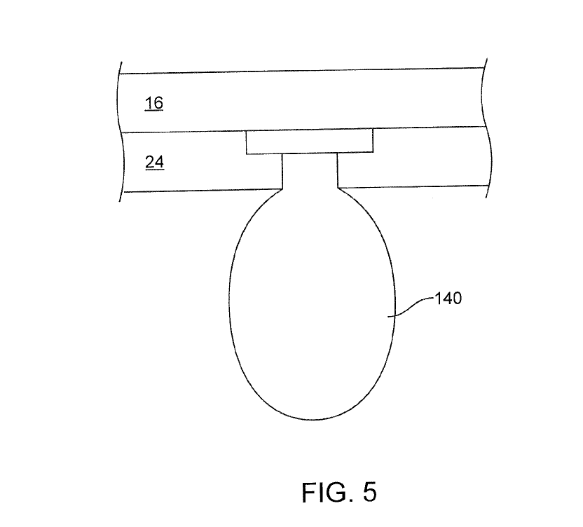

FIG. 5 shows a therapeutic device 100 comprising reservoir 140 suitable for loading in a cannula, in which the reservoir 140 comprises an expanded configuration.

Examples of therapeutic agents 110 that may be delivered by the therapeutic device 100 are described in Table 1A and may include Triamcinolone acetonide, Bimatoprost (Lumigan), Ranibizumab (Lucentis™), Travoprost (Travatan, Alcon), Timolol (Timoptic, Merck), Levobunalol (Betagan, Allergan), Brimonidine (Alphagan, Allergan), Dorzolamide (Trusopt, Merck), Brinzolamide (Azopt, Alcon). Additional examples of therapeutic agents that may be delivered by the therapeutic device include antibiotics such as tetracycline, chlortetracycline, bacitracin, neomycin, polymyxin, gramicidin, cephalexin, oxytetracycline, chloramphenicol kanamycin, rifampicin, ciprofloxacin, tobramycin, gentamycin, erythromycin and penicillin; antifungals such as amphotericin B and miconazole; anti-bacterials such as sulfonamides, sulfadiazine, sulfacetamide, sulfamethizole and sulfisoxazole, nitrofurazone and sodium propionate; antivirals such as idoxuridine, trifluorotymidine, acyclovir, ganciclovir and interferon; antiallergenics such as sodium cromoglycate, antazoline, methapyriline, chlorpheniramine, pyrilamine, cetirizine and prophenpyridamine; anti-inflammatories such as hydrocortisone, hydrocortisone acetate, dexamethasone, dexamethasone 21-phosphate, fluocinolone, medrysone, prednisolone, prednisolone 21-phosphate, prednisolone acetate, fluoromethalone, betamethasone, and triamcinolone; non-steroidal anti-inflammatories such as salicylate, indomethacin, ibuprofen, diclofenac, flurbiprofen and piroxicam; decongestants such as phenylephrine, naphazoline and tetrahydrozoline; miotics and anticholinesterases such as pilocarpine, salicylate, acetylcholine chloride, physostigmine, eserine, carbachol, diisopropyl fluorophosphate, phospholine iodide and demecarium bromide; mydriatics such as atropine sulfate, cyclopentolate, homatropine, scopolamine, tropicamide, eucatropine and hydroxyamphetamine; sypathomimetics such as epinephrine; antineoplastics such as carmustine, cisplatin and fluorouracil; immunological drugs such as vaccines and immune stimulants; hormonal agents such as estrogens, estradiol, progestational, progesterone, insulin, calcitonin, parathyroid hormone and peptide and vasopressin hypothalamus releasing factor; beta adrenergic blockers such as timolol maleate, levobunolol Hcl and betaxolol Hcl; growth factors such as epidermal growth factor, fibroblast growth factor, platelet derived growth factor, transforming growth factor beta, somatotropin and fibronectin; carbonic anhydrase inhibitors such as dichlorophenamide, acetazolamide and methazolamide and other drugs such as prostaglandins, antiprostaglandins and prostaglandin precursors. Other therapeutic agents known to those skilled in the art which are capable of controlled, sustained release into the eye in the manner described herein are also suitable for use in accordance with embodiments described herein.

The therapeutic agent 110 may comprise one or more of the following: Abarelix, Abatacept, Abciximab, Adalimumab, Aldesleukin, Alefacept, Alemtuzumab, Alpha-1-proteinase inhibitor, Alteplase, Anakinra, Anistreplase, Antihemophilic Factor, Antithymocyte globulin, Aprotinin, Arcitumomab, Asparaginase, Basiliximab, Becaplermin, Bevacizumab, Bivalirudin, Botulinum Toxin Type A, Botulinum Toxin Type B, Capromab, Cetrorelix, Cetuximab, Choriogonadotropin alfa, Coagulation Factor IX, Coagulation factor VIIa, Collagenase, Corticotropin, Cosyntropin, Cyclosporine, Daclizumab, Darbepoetin alfa, Defibrotide, Denileukin diftitox, Desmopressin, Dornase Alfa, Drotrecogin alfa, Eculizumab, Efalizumab, Enfuvirtide, Epoetin alfa, Eptifibatide, Etanercept, Exenatide, Felypressin, Filgrastim, Follitropin beta, Galsulfase, Gemtuzumab ozogamicin, Glatiramer Acetate, Glucagon recombinant, Goserelin, Human Serum Albumin, Hyaluronidase, Ibritumomab, Idursulfase, Immune globulin, Infliximab, Insulin Glargine recombinant, Insulin Lyspro recombinant, Insulin recombinant, Insulin, porcine, Interferon Alfa-2a, Recombinant, Interferon Alfa-2b, Recombinant, Interferon alfacon-1, Interferon alfa-n1, Interferon alfa-n3, Interferon beta-1b, Interferon gamma-1b, Lepirudin, Leuprolide, Lutropin alfa, Mecasermin, Menotropins, Muromonab, Natalizumab, Nesiritide, Octreotide, Omalizumab, Oprelvekin, OspA lipoprotein, Oxytocin, Palifermin, Palivizumab, Panitumumab, Pegademase bovine, Pegaptanib, Pegaspargase, Pegfilgrastim, Peginterferon alfa-2a, Peginterferon alfa-2b, Pegvisomant, Pramlintide, Ranibizumab, Rasburicase, Reteplase, Rituximab, Salmon Calcitonin, Sargramostim, Secretin, Sermorelin, Serum albumin iodonated, Somatropin recombinant, Streptokinase, Tenecteplase, Teriparatide, Thyrotropin Alfa, Tositumomab, Trastuzumab, Urofollitropin, Urokinase, or Vasopressin. The molecular weights of the molecules and indications of these therapeutic agents are set for below in Table 1A, below.

The therapeutic agent 110 may comprise one or more of compounds that act by binding members of the immunophilin family of cellular proteins. Such compounds are known as “immunophilin binding compounds.” Immunophilin binding compounds include but are not limited to the “limus” family of compounds. Examples of limus compounds that may be used include but are not limited to cyclophilins and FK506-binding proteins (FKBPs), including sirolimus (rapamycin) and its water soluble analog SDZ-RAD, tacrolimus, everolimus, pimecrolimus, CCI-779 (Wyeth), AP23841 (Ariad), and ABT-578 (Abbott Laboratories).

The limus family of compounds may be used in the compositions, devices and methods for the treatment, prevention, inhibition, delaying the onset of, or causing the regression of angiogenesis-mediated diseases and conditions of the eye, including choroidal neovascularization. The limus family of compounds may be used to prevent, treat, inhibit, delay the onset of, or cause regression of AMD, including wet AMD. Rapamycin may be used to prevent, treat, inhibit, delay the onset of, or cause regression of angiogenesis-mediated diseases and conditions of the eye, including choroidal neovascularization. Rapamycin may be used to prevent, treat, inhibit, delay the onset of, or cause regression of AMD, including wet AMD.

The therapeutic agent 110 may comprise one or more of: pyrrolidine, dithiocarbamate (NF.kappa.B inhibitor); squalamine; TPN 470 analogue and fumagillin; PKC (protein kinase C) inhibitors; Tie-1 and Tie-2 kinase inhibitors; inhibitors of VEGF receptor kinase; proteosome inhibitors such as Velcade™ (bortezomib, for injection; ranibuzumab (Lucentis™ and other antibodies directed to the same target; pegaptanib (Macugen™; vitronectin receptor antagonists, such as cyclic peptide antagonists of vitronectin receptor-type integrins; .alpha.-v/.beta.-3 integrin antagonists; .alpha.-v/.beta.-1 integrin antagonists; thiazolidinediones such as rosiglitazone or troglitazone; interferon, including .gamma.-interferon or interferon targeted to CNV by use of dextran and metal coordination; pigment epithelium derived factor (PEDF); endostatin; angiostatin; tumistatin; canstatin; anecortave acetate; acetonide; triamcinolone; tetrathiomolybdate; RNA silencing or RNA interference (RNAi) of angiogenic factors, including ribozymes that target VEGF expression; Accutane™ (13-cis retinoic acid); ACE inhibitors, including but not limited to quinopril, captopril, and perindozril; inhibitors of mTOR (mammalian target of rapamycin); 3-aminothalidomide; pentoxifylline; 2-methoxyestradiol; colchicines; AMG-1470; cyclooxygenase inhibitors such as nepafenac, rofecoxib, diclofenac, rofecoxib, NS398, celecoxib, vioxx, and (E)-2-alkyl-2(4-methanesulfonylphenyl)-1-phenylethene; t-RNA synthase modulator; metalloprotease 13 inhibitor; acetylcholinesterase inhibitor; potassium channel blockers; endorepellin; purine analog of 6-thioguanine; cyclic peroxide ANO-2; (recombinant) arginine deiminase; epigallocatechin-3-gallate; cerivastatin; analogues of suramin; VEGF trap molecules; apoptosis inhibiting agents; Visudyne™, snET2 and other photo sensitizers, which may be used with photodynamic therapy (PDT); inhibitors of hepatocyte growth factor (antibodies to the growth factor or its receptors, small molecular inhibitors of the c-met tyrosine kinase, truncated versions of HGF e.g., NK4).

The therapeutic agent 110 may comprise a combination with other therapeutic agents and therapies, including but not limited to agents and therapies useful for the treatment of angiogenesis or neovascularization, particularly CNV. Non-limiting examples of such additional agents and therapies include pyrrolidine, dithiocarbamate (NF.kappa.B inhibitor); squalamine; TPN 470 analogue and fumagillin; PKC (protein kinase C) inhibitors; Tie-1 and Tie-2 kinase inhibitors; inhibitors of VEGF receptor kinase; proteosome inhibitors such as Velcade™ (bortezomib, for injection; ranibuzumab (Lucentis™) and other antibodies directed to the same target; pegaptanib (Macugen™); vitronectin receptor antagonists, such as cyclic peptide antagonists of vitronectin receptor-type integrins; .alpha.-v/.beta.-3 integrin antagonists; .alpha.-v/.beta.-1 integrin antagonists; thiazolidinediones such as rosiglitazone or troglitazone; interferon, including .gamma.-interferon or interferon targeted to CNV by use of dextran and metal coordination; pigment epithelium derived factor (PEDF); endostatin; angiostatin; tumistatin; canstatin; anecortave acetate; acetonide; triamcinolone; tetrathiomolybdate; RNA silencing or RNA interference (RNAi) of angiogenic factors, including ribozymes that target VEGF expression; Accutane™ (13-cis retinoic acid); ACE inhibitors, including but not limited to quinopril, captopril, and perindozril; inhibitors of mTOR (mammalian target of rapamycin); 3-aminothalidomide; pentoxifylline; 2-methoxyestradiol; colchicines; AMG-1470; cyclooxygenase inhibitors such as nepafenac, rofecoxib, diclofenac, rofecoxib, NS398, celecoxib, vioxx, and (E)-2-alkyl-2(4-methanesulfonylphenyl)-1-phenylethene; t-RNA synthase modulator; metalloprotease 13 inhibitor; acetylcholinesterase inhibitor; potassium channel blockers; endorepellin; purine analog of 6-thioguanine; cyclic peroxide ANO-2; (recombinant) arginine deiminase; epigallocatechin-3-gallate; cerivastatin; analogues of suramin; VEGF trap molecules; inhibitors of hepatocyte growth factor (antibodies to the growth factor or its receptors, small molecular inhibitors of the c-met tyrosine kinase, truncated versions of HGF e.g., NK4); apoptosis inhibiting agents; Visudyne™ snET2 and other photo sensitizers with photodynamic therapy (PDT); and laser photocoagulation.

The therapeutic agents may be used in conjunction with a pharmaceutically acceptable carrier such as, for example, solids such as starch, gelatin, sugars, natural gums such as acacia, sodium alginate and carboxymethyl cellulose; polymers such as silicone rubber; liquids such as sterile water, saline, dextrose, dextrose in water or saline; condensation products of castor oil and ethylene oxide, liquid glyceryl triester of a lower molecular weight fatty acid; lower alkanols; oils such as corn oil, peanut oil, sesame oil, castor oil, and the like, with emulsifiers such as mono- or di-glyceride of a fatty acid, or a phosphatide such as lecithin, polysorbate 80, and the like; glycols and polyalkylene glycols; aqueous media in the presence of a suspending agent, for example, sodium carboxymethylcellulose, sodium hyaluronate, sodium alginate, poly(vinyl pyrrolidone) and similar compounds, either alone, or with suitable dispensing agents such as lecithin, polyoxyethylene stearate and the like. The carrier may also contain adjuvants such as preserving, stabilizing, wetting, emulsifying agents or other related materials.

The therapeutic device may comprise a container configured to hold at least one therapeutic agent, the container comprising a chamber to hold the at least one therapeutic agent with at least one opening to release the at least one therapeutic agent to the vitreous humor and porous structure 150 placed within the at least one opening. The porous structure 150 may comprise a fixed tortuous, porous material such as a sintered metal, a sintered glass or a sintered polymer with a defined porosity and tortuosity that controls the rate of delivery of the at least one therapeutic agent to the vitreous humor. The rigid porous structures provide certain advantages over capillary tubes, erodible polymers and membranes as a mechanism for controlling the release of a therapeutic agent or agents from the therapeutic device. These advantages include the ability of the rigid porous structure to comprise a needle stop, simpler and more cost effective manufacture, flushability for cleaning or declogging either prior to or after implantation, high efficiency depth filtration of microorganisms provided by the labyrinths of irregular paths within the structure and greater robustness due to greater hardness and thickness of the structure compared to a membrane or erodible polymer matrix. Additionally, when the rigid porous structure is manufactured from a sintered metal, ceramic, glass or certain plastics, it can be subjected to sterilization and cleaning procedures, such as heat or radiation based sterilization and depyrogenation, that might damage polymer and other membranes. In many embodiments, the rigid porous structure may be configured to provide a therapeutically effective, concentration of the therapeutic agent in the vitreous for at least 6 months. This release profile provided by certain configurations of the rigid porous structures enables a smaller device which is preferred in a small organ such as the eye where larger devices may alter or impair vision.

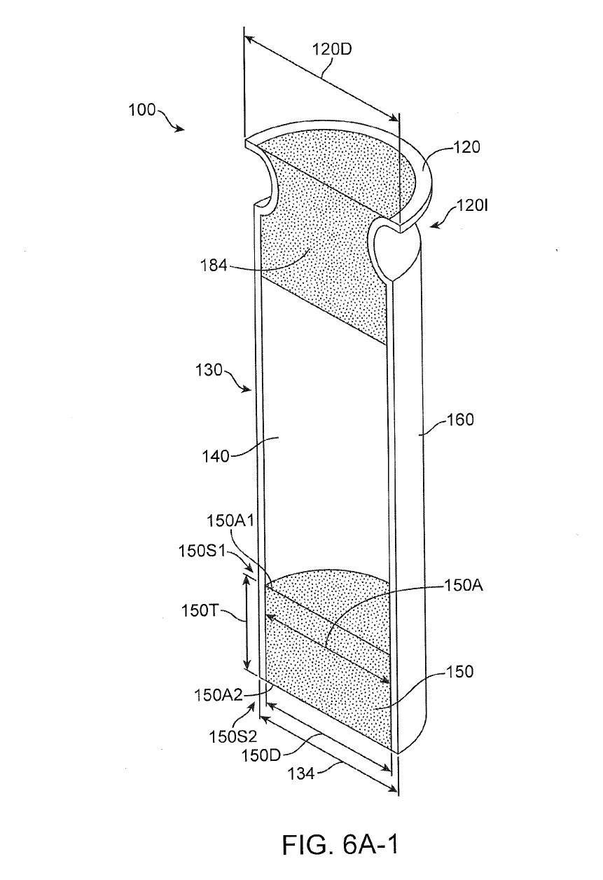

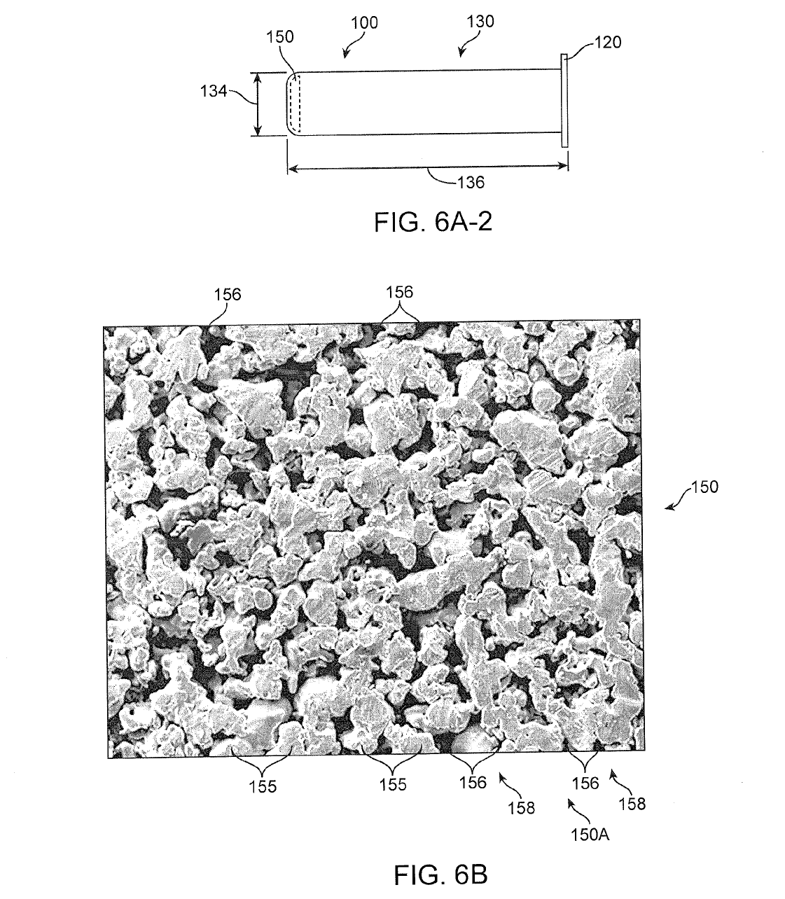

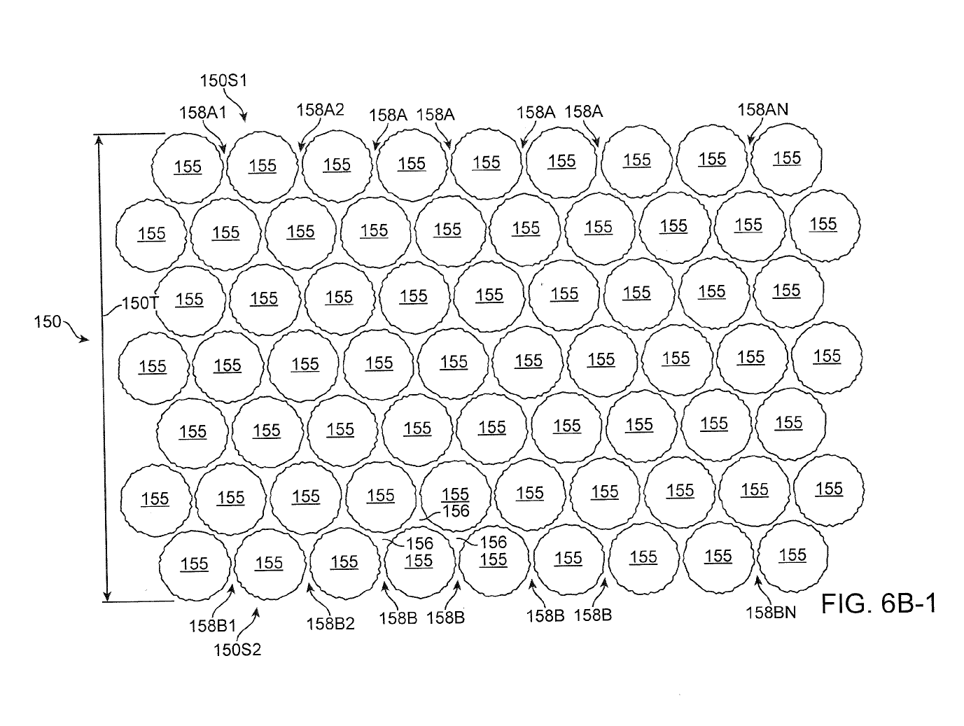

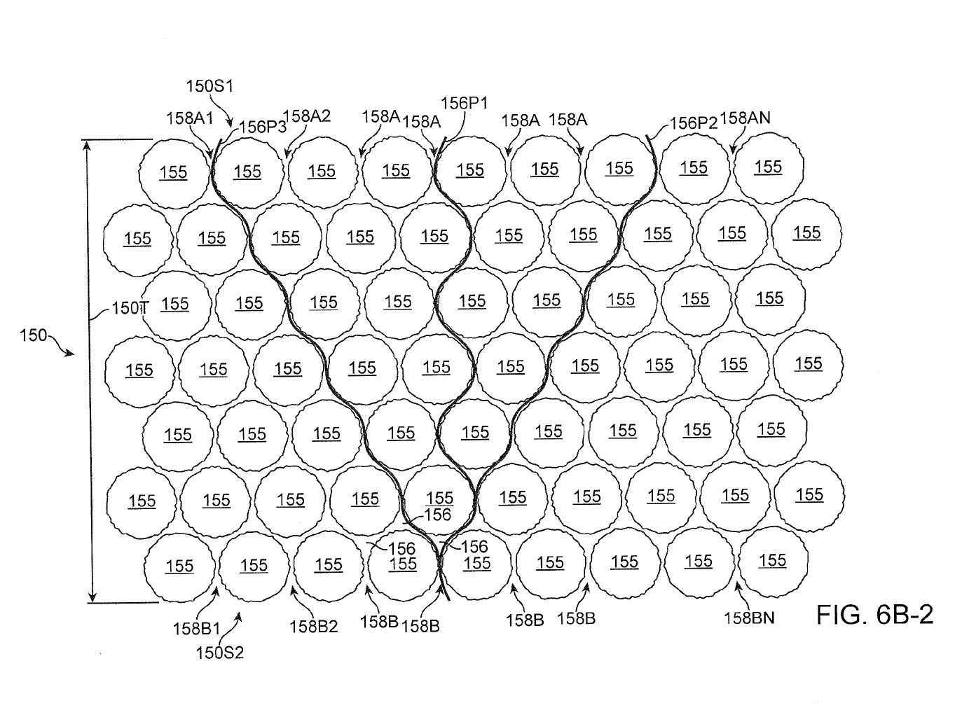

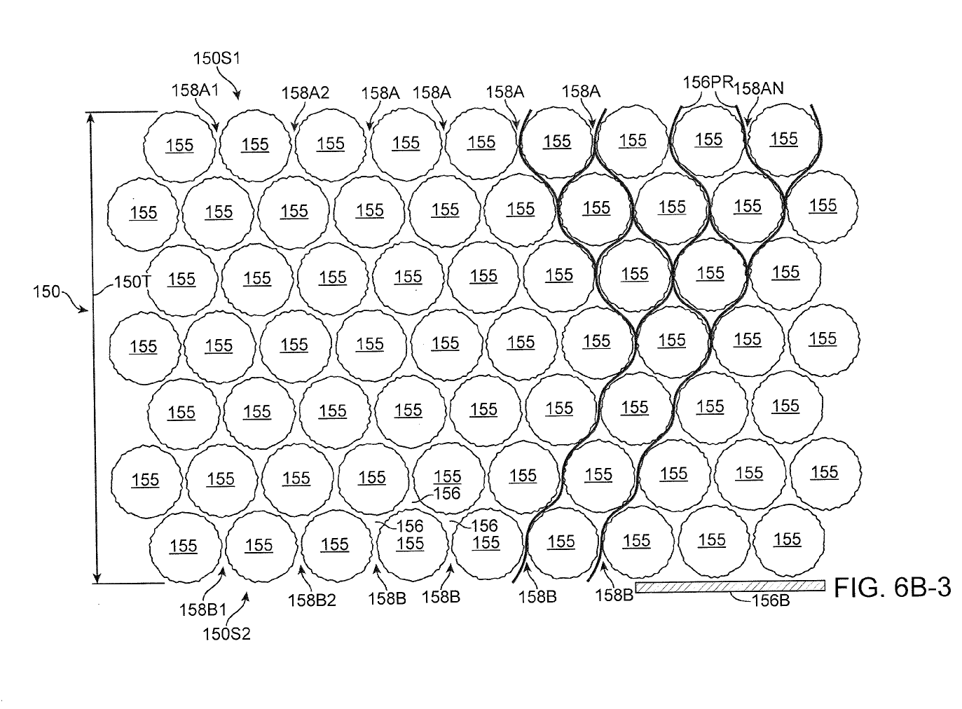

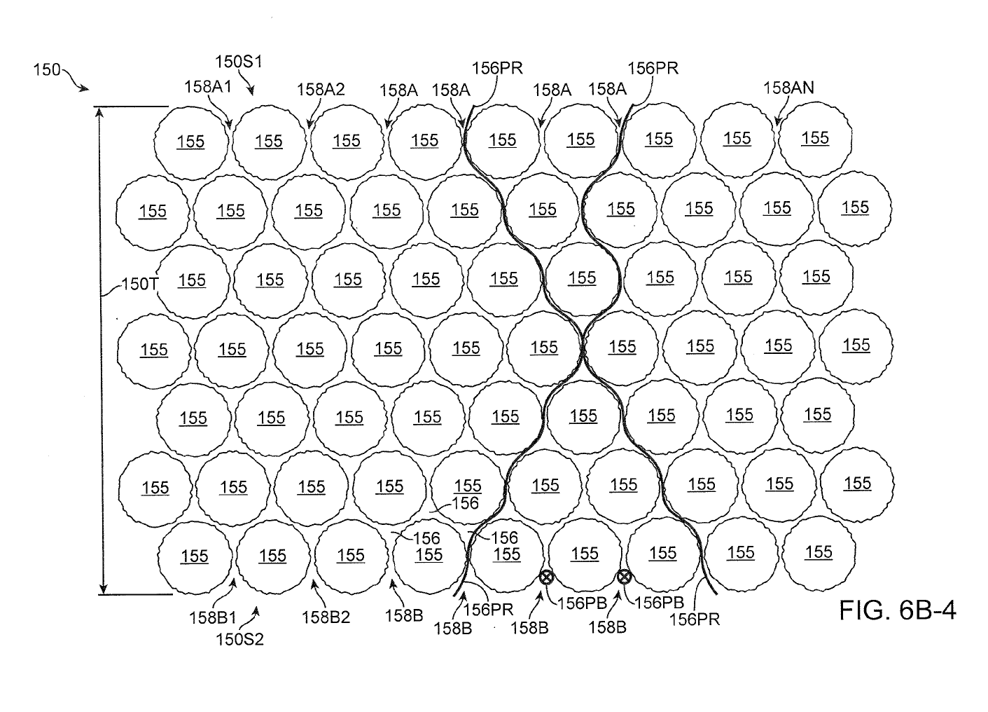

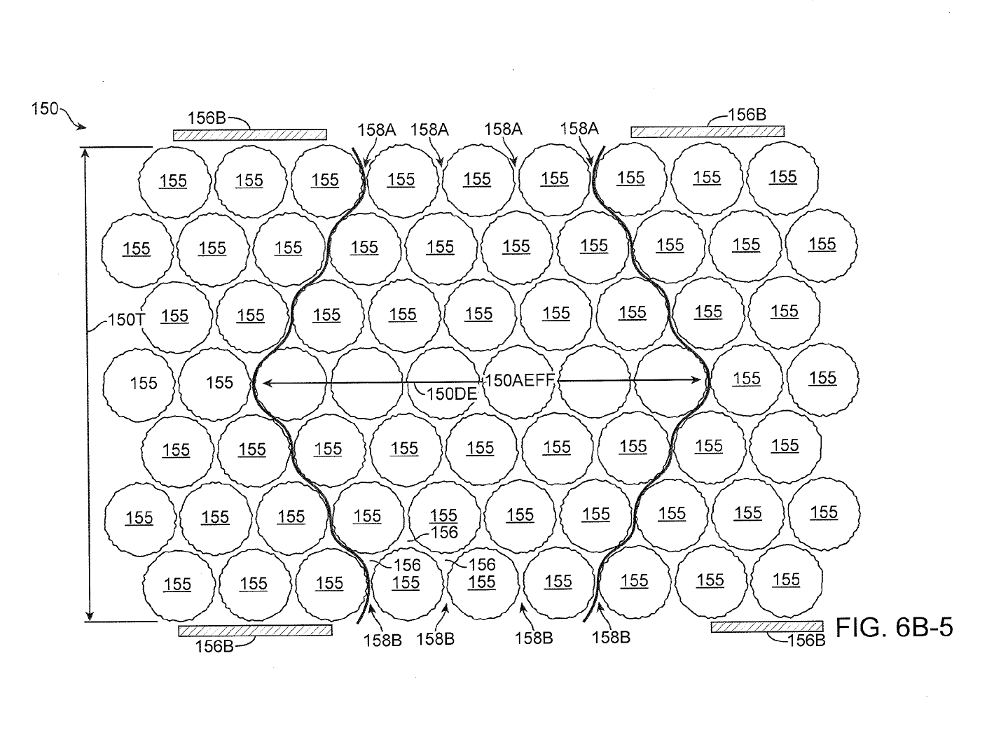

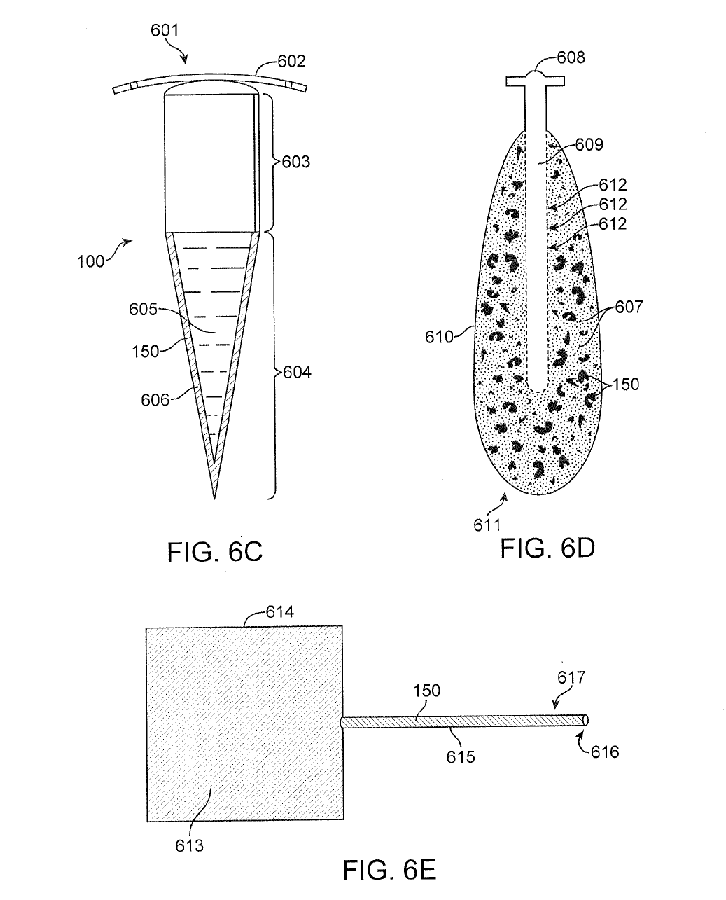

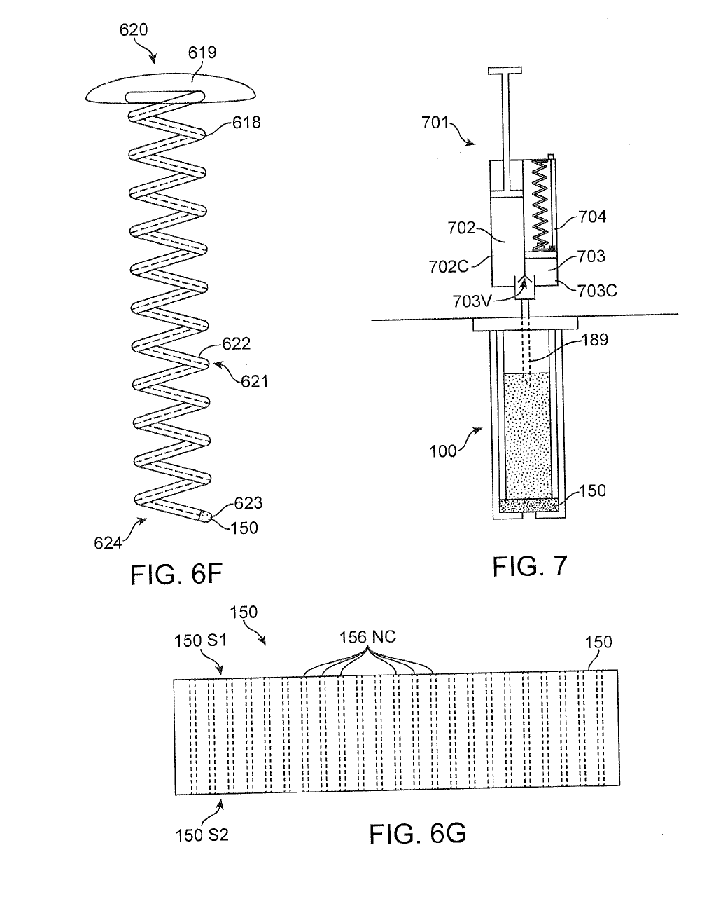

FIG. 6A1 shows a therapeutic device 100 comprising a container 130 having a penetrable barrier 184 disposed on a first end, a porous structure 150 disposed on a second end to release therapeutic agent for an extended period, and a retention structure 120 comprising an extension protruding outward from the container to couple to the sclera and the conjunctiva. The extending protrusion of the retention structure may comprise a diameter 120D. The retention structure may comprise an indentation 1201 sized to receive the sclera. The container may comprise a tubular barrier 160 that defines at least a portion of the reservoir, and the container may comprise a width, for example a diameter 134. The diameter 134 can be sized within a range, for example within a range from about 0.5 to about 4 mm, for example within a range from about 1 to 3 mm and can be about 2 mm, for example. The container may comprise a length 136, sized so as to extend from the conjunctive to the vitreous to release the therapeutic agent into the vitreous. The length 136 can be sized within a range, for example within a range from about 2 to about 14 mm, for example within a range from about 4 to 10 mm and can be about 7 mm, for example. The volume of the reservoir may be substantially determined by an inner cross-sectional area of the tubular structure and distance from the porous structure to the penetrable barrier. The retention structure may comprise an annular extension having a retention structure diameter greater than a diameter of the container. The retention structure may comprise an indentation configured to receive the sclera when the extension extends between the sclera and the conjunctiva. The penetrable barrier may comprise a septum disposed on a proximal end of the container, in which the septum comprises a barrier that can be penetrated with a sharp object, such as a needle for injection of the therapeutic agent. The porous structure may comprise a cross-sectional area 150A sized to release the therapeutic agent for the extended period.

The porous structure 150 may comprise a first side 150S1 coupled to the reservoir and a second side 150S2 to couple to the vitreous. The first side may comprise a first area 150A1 and the second side may comprise a second area 150A2. The porous structure may comprise a thickness 105T. The porous structure may comprise a diameter 150D.

The volume of the reservoir 140 may comprise from about 5 uL to about 2000 uL of therapeutic agent, or for example from about 10 uL to about 200 uL of therapeutic agent.

The therapeutic agent stored in the reservoir of the container comprises at least one of a solid comprising the therapeutic agent, a solution comprising the therapeutic agent, a suspension comprising the therapeutic agent, particles comprising the therapeutic agent adsorbed thereon, or particles reversibly bound to the therapeutic agent. For example, reservoir may comprise a suspension of a cortico-steroid such as triamcinolone acetonide to treat inflammation of the retina. The reservoir may comprise a buffer and a suspension of a therapeutic agent comprising solubility within a range from about 1 ug/mL to about 100 ug/mL, such as from about 1 ug/mL to about 40 ug/mL. For example, the therapeutic agent may comprise a suspension of triamcinolone acetonide having a solubility of approximately 19 ug/mL in the buffer at 37 degrees Centigrade when implanted.

The release rate index may comprise many values, and the release rate index with the suspension may be somewhat higher than for a solution in many embodiments, for example. The release rate index may be no more than about 5, and can be no more than about 2.0, for example no more than about 1.5, and in many embodiments may be no more than about 1.2, so as to release the therapeutic agent with therapeutic amounts for the extended time.

The therapeutic device, including for example, the retention structure and the porous structure, may be sized to pass through a lumen of a catheter.

The porous structure may comprise a needle stop that limits penetration of the needle. The porous structure may comprise a plurality of channels configured for the extended release of the therapeutic agent. The porous structure may comprise a rigid sintered material having characteristics suitable for the sustained release of the material.

FIG. 6A2 shows a therapeutic device as in FIG. 6A comprising a rounded distal end.

FIG. 6B shows a rigid porous structure as in FIG. 6A. The rigid porous structure 158 comprises a plurality of interconnecting channels 156. The porous structure comprises a sintered material composed of interconnected grains 155 of material. The interconnected grains of material define channels that extend through the porous material to release the therapeutic agent. The channels may extend around the sintered grains of material, such that the channels comprise interconnecting channels extending through the porous material.

The rigid porous structure can be configured for injection of the therapeutic agent into the container in many ways. The channels of the rigid porous structure may comprise substantially fixed channels when the therapeutic agent is injected into the reservoir with pressure. The rigid porous structure comprises a hardness parameter within a range from about 160 Vickers to about 500 Vickers. In some embodiments, the rigid porous structure is formed from sintered stainless steel and comprises a hardness parameter within a range from about 200 Vickers to about 240 Vickers. In some embodiments, it is preferred to inhibit ejection of the therapeutic agent through the porous structure during filling or refilling the reservoir of the therapeutic device with a fluid. In these embodiments, the channels of the rigid porous structure comprise a resistance to flow of an injected solution or suspension through a thirty gauge needle such that ejection of said solution or suspension through the rigid porous structure is substantially inhibited when said solution or suspension is injected into the reservoir of the therapeutic device. Additionally, these embodiments may optionally comprise an evacuation vent or an evacuation reservoir under vacuum or both to facilitate filling or refilling of the reservoir.

The reservoir and the porous structure can be configured to release therapeutic amounts of the therapeutic agent in many ways. The reservoir and the porous structure can be configured to release therapeutic amounts of the therapeutic agent corresponding to a concentration of at least about 0.1 ug per ml of vitreous humor for an extended period of at least about three months. The reservoir and the porous structure can be configured to release therapeutic amounts of the therapeutic agent corresponding to a concentration of at least about 0.1 ug per ml of vitreous humor and no more than about 10 ug per ml for an extended period of at least about three months. The therapeutic agent may comprise at least a fragment of an antibody and a molecular weight of at least about 10 k Daltons. For example, the therapeutic agent may comprise one or more of ranibizumab or bevacizumab. Alternatively or in combination, the therapeutic agent may comprise a small molecule drug suitable for sustained release. The reservoir and the porous structure may be configured to release therapeutic amounts of the therapeutic agent corresponding to a concentration of at least about 0.1 ug per ml of vitreous humor and no more than about 10 ug per ml for an extended period of at least about 3 months or at least about 6 months. The reservoir and the porous structure can be configured to release therapeutic amounts of the therapeutic agent corresponding to a concentration of at least about 0.1 ug per ml of vitreous humor and no more than about 10 ug per ml for an extended period of at least about twelve months or at least about two years or at least about three years. The reservoir and the porous structure may also be configured to release therapeutic amounts of the therapeutic agent corresponding to a concentration of at least about 0.01 ug per ml of vitreous humor and no more than about 300 ug per ml for an extended period of at least about 3 months or 6 months or 12 months or 24 months.

The channels of the rigid porous structure comprise a hydrogel configured to limit a size of molecules passed through the channels of the rigid porous structure. For example, the hydrogel can be formed within the channels and may comprise an acrylamide gel. The hydrogel comprises a water content of at least about 70%. For example, the hydrogel may comprise a water content of no more than about 90% to limit molecular weight of the therapeutic agent to about 30 k Daltons. The hydrogel comprises a water content of no more than about 95% to limit molecular weight of the therapeutic agent to about 100 k Daltons. The hydrogel may comprise a water content within a range from about 90% to about 95% such that the channels of the porous material are configured to pass Lucentis™ and substantially not pass Avastin™.

The rigid porous structure may comprise a composite porous material that can readily be formed in or into a wide range of different shapes and configurations. For example, the porous material can be a composite of a metal, aerogel or ceramic foam (i.e., a reticulated inter-cellular structure in which the interior cells are interconnected to provide a multiplicity of pores passing through the volume of the structure, the walls of the cells themselves being substantially continuous and non-porous, and the volume of the cells relative to that of the material forming the cell walls being such that the overall density of the intercellular structure is less than about 30 percent theoretical density) through pores of which are impregnated with a sintered powder or aerogel. The thickness, density, porosity and porous characteristics of the final composite porous material can be varied to conform with the desired release of the therapeutic agent.

Embodiments comprise a method of making an integral (i.e., single-component) porous structure. The method may comprise introducing particles into a mold having a desired shape for the porous structure. The shape includes a proximal end defining a plurality of proximal porous channel openings to couple to the reservoir, a distal end defining a plurality of outlet channel openings to couple to the vitreous humor of the eye, a plurality of blind inlet cavities extending into the filter from the proximal openings, and a plurality of blind outlet cavities extending into the porous structure from the outlet channel openings. The method further includes applying pressure to the mold, thereby causing the particles to cohere and form a single component, and sintering the component to form the porous structure. The particles can be pressed and cohere to form the component without the use of a polymeric binder, and the porous structure can be formed substantially without machining.

The mold can be oriented vertically with the open other end disposed upwardly, and metal powder having a particle size of less than 20 micrometers can be introduced into the cavity through the open end of the mold while vibrating the mold to achieve substantially uniform packing of the metal powder in the cavity. A cap can be placed on the open other end of the mold, and pressure is applied to the mold and thereby to the metal powder in the cavity to cause the metal powder to cohere and form a cup-shaped powdered metal structure having a shape corresponding to the mold. The shaped powdered metal structure can be removed from the mold, and sintered to obtain a porous sintered metal porous structure.

The metal porous structure can be incorporated into the device by a press fit into an impermeable structure with an opening configured to provide a tight fit with the porous structure. Other means, such as welding, known to those skilled in the art can be used to incorporate the porous structure into the device. Alternatively, or in combination, the powdered metal structure can be formed in a mold where a portion of the mold remains with the shaped powdered metal structure and becomes part of the device. This may be advantageous in achieving a good seal between the porous structure and the device.

The release rate of therapeutic agent through a porous body, such as a sintered porous metal structure or a porous glass structure, may be described by diffusion of the therapeutic agent within the porous structure with the channel parameter, and with an effective diffusion coefficient equal to the diffusion coefficient of the therapeutic agent in the liquid that fills the reservoir multiplied by the Porosity and a Channel Parameter of the porous body:

Release Rate=(D P/F)A(c_{R}−c_{V})/L, where:
c_{R}=Concentration in reservoir
c_{V}=Concentration outside of the reservoir or in the vitreous
D=Diffusion coefficient of the therapeutic agent in the reservoir solution
P=Porosity of porous structure
F=Channel parameter that may correspond to a tortuosity parameter of channels of porous structure
A=Area of porous structure
L=Thickness (length) of porous structure

Cumulative Release=1−cR/cR0=1−exp((−D PA/FL V_{R})t), where
t=time, Vr=reservoir volume

The release rate index can (hereinafter RRI) be used to determine release of the therapeutic agent. The RRI may be defined as (PA/FL), and the RRI values herein will have units of mm unless otherwise indicated. Many of the porous structures used in the therapeutic delivery devices described here have an RRI of no more than about 5.0, often no more than about 2.0, and can be no more than about 1.2 mm.

The channel parameter can correspond to an elongation of the path of the therapeutic agent released through the porous structure. The porous structure may comprise many interconnecting channels, and the channel parameter can correspond to an effective length that the therapeutic agent travels along the interconnecting channels of the porous structure from the reservoir side to the vitreous side when released. The channel parameter multiplied by the thickness (length) of the porous structure can determine the effective length that the therapeutic agent travels along the interconnecting channels from the reservoir side to the vitreous side. For example, the channel parameter (F) of about 1.5 corresponds to interconnecting channels that provide an effective increase in length traveled by the therapeutic agent of about 50%, and for a 1 mm thick porous structure the effective length that the therapeutic agent travels along the interconnecting channels from the reservoir side to the vitreous side corresponds to about 1.5 mm. The channel parameter (F) of at least about 2 corresponds to interconnecting channels that provide an effective increase in length traveled by the therapeutic agent of about 100%, and for a 1 mm thick porous structure the effective length that the therapeutic agent travels along the interconnecting channels from the reservoir side to the vitreous side corresponds to at least about 2.0 mm. As the porous structure comprises many interconnecting channels that provide many alternative paths for release of the therapeutic agent, blockage of some of the channels provides no substantial change in the effective path length through the porous structure as the alternative interconnecting channels are available, such that the rate of diffusion through the porous structure and the release of the therapeutic agent are substantially maintained when some of the channels are blocked.

If the reservoir solution is aqueous or has a viscosity similar to water, the value for the diffusion coefficient of the therapeutic agent (TA) in water at the temperature of interest may be used. The following equation can be used to estimate the diffusion coefficient at 37° C. from the measured value of D_{BSA,20C}=6.1 e-7 cm2/s for bovine serum albumin in water at 20° C. (Molokhia et al, Exp Eye Res 2008):

D_{TA,37C}=D_{BSA,20C}(η_{20C}/η_{37C})(MW_{BSA}/MW_{TA})^{1/3} where

MW refers to the molecular weight of either BSA or the test compound and η is the viscosity of water. The following lists diffusion coefficients of proteins of interest.

| | | | Diff Coeff |
|---|---|---|---|
| Compound | MW | Temp C. | (cm²/s) |
| BSA | 69,000 | 20 | 6.1E−07 |
| BSA | 69,000 | 37 | 9.1E−07 |
| Ranibizumab | 48,000 | 20 | 6.9E−07 |
| Ranibizumab | 48,000 | 37 | 1.0E−06 |
| Bevacizumab | 149,000 | 20 | 4.7E−07 |
| Bevacizumab | 149,000 | 37 | 7.1E−07 |

Small molecules have a diffusion coefficient similar to fluorescein (MW=330, D=4.8 to 6 e-6 cm²/s from Stay, M S et al., Pharm Res 2003, 20(1), pp. 96-102). For example, the small molecule may comprise a glucocorticoid such as triamcinolone acetonide having a molecular weight of about 435.

The porous structure comprises a porosity, a thickness, a channel parameter and a surface area configured to release therapeutic amounts for the extended period. The porous material may comprise a porosity corresponding to the fraction of void space of the channels extending within the material. The porosity comprises a value within a range from about 3% to about 70%. In other embodiments, the porosity comprises a value with a range from about 5% to about 10% or from about 10% to about 25%, or for example from about 15% to about 20%. Porosity can be determined from the weight and macroscopic volume or can be measured via nitrogen gas adsorption

The porous structure may comprise a plurality of porous structures, and the area used in the above equation may comprise the combined area of the plurality of porous structures.

The channel parameter may comprise a fit parameter corresponding to the tortuosity of the channels. For a known porosity, surface area and thickness of the surface parameter, the curve fit parameter F, which may correspond to tortuosity of the channels can be determined based on experimental measurements. The parameter PA/FL can be used to determine the desired sustained release profile, and the values of P, A, F and L determined. The rate of release of the therapeutic agent corresponds to a ratio of the porosity to the channel parameter, and the ratio of the porosity to the channel parameter can be less than about 0.5 such that the porous structure releases the therapeutic agent for the extended period. For example, the ratio of the porosity to the channel parameter is less than about 0.1 or for example less than about 0.2 such that the porous structure releases the therapeutic agent for the extended period. The channel parameter may comprise a value of at least about 1, such as at least about 1.2. For example, the value of the channel parameter may comprise at least about 1.5, for example at least about 2, and may comprise at least about 5. The channel parameter can be within a range from about 1.1 to about 10, for example within a range from about 1.2 to about 5. A person of ordinary skill in the art can conduct experiments based on the teachings described herein to determine empirically the channel parameter to release the therapeutic agent for an intended release rate profile.

The area in the model originates from the description of mass transported in units of flux; i.e., rate of mass transfer per unit area. For simple geometries, such as a porous disc mounted in an impermeable sleeve of equal thickness, the area corresponds to one face of the disc and the thickness, L, is the thickness of the disc. For more complex geometries, such as a porous body in the shape of a truncated cone, the effective area is a value in between the area where therapeutic agent enters the porous body and the area where therapeutic agent exits the porous body.

A model can be derived to describe the release rate as a function of time by relating the change of concentration in the reservoir to the release rate described above. This model assumes a solution of therapeutic agent where the concentration in the reservoir is uniform. In addition, the concentration in the receiving fluid or vitreous is considered negligible (c_{V}=0). Solving the differential equation and rearrangement yields the following equations describing the concentration in the reservoir as a function of time, t, and volume of the reservoir, V_{R}, for release of a therapeutic agent from a solution in a reservoir through a porous structure.

c_{R}=c_{R0} exp((−D PA/FL V_{R})t)

and Cumulative Release=1−c_{R}/c_{R0}

When the reservoir contains a suspension, the concentration in reservoir, c_{R}, is the dissolved concentration in equilibrium with the solid (i.e., the solubility of the therapeutic agent). In this case, the concentration in the reservoir is constant with time, the release rate is zero order, and the cumulative release increases linearly with time until the time when the solid is exhausted.

Therapeutic concentrations for many ophthalmic therapeutic agents may be determined experimentally by measuring concentrations in the vitreous humor that elicit a therapeutic effect. Therefore, there is value in extending predictions of release rates to predictions of concentrations in the vitreous. A one-compartment model may be used to describe elimination of therapeutic agent from eye tissue.

Current intravitreal administration of therapeutic agents such as Lucentis™ involves a bolus injection. A bolus injection into the vitreous may be modeled as a single exponential with rate constant, k=0.693/half-life and a cmax=dose/Vᵥ where Vᵥ is the vitreous volume. As an example, the half-life for ranibizumab is approximately 3 days in the rabbit and the monkey (Gaudreault et al) and 9 days in humans (Lucentis™ package insert). The vitreous volume is approximately 1.5 mL for the rabbit and monkey and 4.5 mL for the human eye. The model predicts an initial concentration of 333 ug/mL for a bolus injection of 0.5 mg Lucentis™ into the eye of a monkey. This concentration decays to a vitreous concentration of 0.1 ug/mL after about a month.

For devices with extended release, the concentration in the vitreous changes slowly with time. In this situation, a model can be derived from a mass balance equating the release rate from the device (described by equations above) with the elimination rate from the eye, k cᵥ Vᵥ. Rearrangement yields the following equation for the concentration in the vitreous:

cᵥ=Release rate from device/k Vᵥ.

Since the release rate from a device with a solution of therapeutic agent decreases exponentially with time, the concentration in the vitreous decreases exponentially with the same rate constant. In other words, vitreous concentration decreases with a rate constant equal to D PA/FL V_{R} and, hence, is dependent on the properties of the porous structure and the volume of the reservoir.

Since the release rate is zero order from a device with a suspension of therapeutic agent, the vitreous concentration will also be time-independent. The release rate will depend on the properties of the porous structure via the ratio, PA/FL, but will be independent of the volume of the reservoir until the time at which the drug is exhausted.

The channels of the rigid porous structure can be sized in many ways to release the intended therapeutic agent. For example, the channels of the rigid porous structure can be sized to pass therapeutic agent comprising molecules having a molecular weight of at least about 100 Daltons or for example, at least about 50 k Daltons. The channels of the rigid porous structure can be sized to pass therapeutic agent comprising molecules comprising a cross-sectional size of no more than about 10 nm. The channels of the rigid porous structure comprise interconnecting channels configured to pass the therapeutic agent among the interconnecting channels. The rigid porous structure comprises grains of rigid material and wherein the interconnecting channels extend at least partially around the grains of rigid material to pass the therapeutic agent through the porous material. The grains of rigid material can be coupled together at a loci of attachment and wherein the interconnecting channels extend at least partially around the loci of attachment.

The porous structure and reservoir may be configured to release the glucocorticoid for an extended time of at least about six months with a therapeutic amount of glucocorticoid of corresponding to an in situ concentration within a range from about 0.05 ug/mL to about 4 ug/mL, for example from 0.1 ug/mL to about 4 ug/mL, so as to suppress inflammation in the retina-choroid.

The porous structure comprises a sintered material. The sintered material may comprise grains of material in which the grains comprise an average size of no more than about 20 um. For example, the sintered material may comprise grains of material in which the grains comprise an average size of no more than about 10 um, an average size of no more than about 5 um, or an average size of no more than about 1 um. The channels are sized to pass therapeutic quantities of the therapeutic agent through the sintered material for the extended time based on the grain size of the sintered material and processing parameters such as compaction force and time and temperature in the furnace. The channels can be sized to inhibit penetration of microbes including bacteria and fungal spores through the sintered material.

The sintered material comprises a wettable material to inhibit bubbles within the channels of the material.

The sintered material comprises at least one of a metal, a ceramic, a glass or a plastic. The sintered material may comprise a sintered composite material, and the composite material comprises two or more of the metal, the ceramic, the glass or the plastic. The metal comprises at least one of Ni, Ti, nitinol, stainless steel including alloys such as 304, 304L, 316 or 316L, cobalt chrome, elgiloy, hastealloy, c-276 alloy or Nickel 200 alloy. The sintered material may comprise a ceramic. The sintered material may comprise a glass. The plastic may comprise a wettable coating to inhibit bubble formation in the channels, and the plastic may comprise at least one of polyether ether ketone (PEEK), polyethylene, polypropylene, polyimide, polystyrene, polycarbonate, polyacrylate, polymethacrylate, or polyamide.

The rigid porous structure may comprise a plurality of rigid porous structures coupled to the reservoir and configured to release the therapeutic agent for the extended period. For example, additional rigid porous structure can be disposed along the container, for example the end of the container may comprise the porous structure, and an additional porous structure can be disposed along a distal portion of the container, for example along a tubular sidewall of the container.

The therapeutic device can be tuned to release therapeutic amounts of the therapeutic agent above the minimum inhibitory concentration for an extended time based on bolus injections of the therapeutic agent. For example, the volume of the chamber of the reservoir can be sized with the release rate of the porous structure based on the volume of the bolus injection. A formulation of a therapeutic agent can be provided, for example a known intravitreal injection formulation. The therapeutic agent can be capable of treating the eye with bolus injections, such that the formulation has a corresponding period between each of the bolus injections to treat the eye. For example, the bolus injections may comprise monthly injections. Each of the bolus injections comprises a volume of the formulation, for example 50 uL. Each of the bolus injections of the therapeutic agent may correspond to a range of therapeutic concentrations of the therapeutic agent within the vitreous humor over the time course between injections, and the device can be tuned so as to release therapeutic amounts of the therapeutic agent such that the vitreous concentrations of the released therapeutic agent from the device are within the range of therapeutic concentrations of the corresponding bolus injections. For example, the therapeutic agent may comprise a minimum inhibitory concentration to treat the eye, for example at least about 3 ug/mL, and the values of the range of therapeutic concentrations can be at least about 3 ug/mL. The therapeutic device can be configured to treat the eye with an injection of the monthly volume of the formulation into the device, for example through the penetrable barrier. The reservoir of the container has a chamber to contain a volume of the therapeutic agent, for example 35 uL, and a mechanism to release the therapeutic agent from the chamber to the vitreous humor.

The volume of the container and the release mechanism can be tuned to treat the eye with the therapeutic agent with vitreous concentrations within the therapeutic range for an extended time with each injection of the quantity corresponding to the bolus injection, such that the concentration of the therapeutic agent within the vitreous humor remains within the range of therapeutic concentrations and comprises at least the minimum inhibitory concentration. The extended time may comprise at least about twice the corresponding period of the bolus injections. The release mechanism comprises one or more of a porous frit, a sintered porous frit, a permeable membrane, a semi-permeable membrane, a capillary tube or a tortuous channel, nano-structures, nano-channels or sintered nano-particles. For example, the porous frit may comprise a porosity, cross sectional area, and a thickness to release the therapeutic agent for the extended time. The volume of the container reservoir can be sized in many ways in relation to the volume of the injected formulation and can be larger than the volume of injected formulation, smaller than the volume of injected formulation, or substantially the same as the volume of injected formulation. For example, the volume of the container may comprise no more than the volume of the formulation, such that at least a portion of the formulation injected into the reservoir passes through the reservoir and comprises a bolus injection to treat the patient immediately. As the volume of the reservoir is increased, the amount of formulation released to the eye through the porous structure upon injection can decrease along with the concentration of active ingredient of the therapeutic agent within the reservoir, and the release rate index can be increased appropriately so as to provide thereapeutic amounts of therapeutic agent for the extended time. For example, the volume of the reservoir of the container can be greater than the volume corresponding to the bolus injection, so as to provide therapeutic amounts for at least about five months, for example 6 months, with an injection volume corresponding to a monthly injection of Lucentis™. For example, the formulation may comprise commercially available Lucentis™, 50 uL, and the reservoir may comprise a volume of about 100 uL and provide therapeutic vitreous concentrations of at least about 3 ug/mL for six months with 50 uL of Lucentis™ injected into the reservoir.

The chamber may comprise a substantially fixed volume and the release rate mechanism comprises a substantially rigid structure to maintain release of the therapeutic agent above the minimum inhibitory concentration for the extended time with each injection of a plurality of injections.

A first portion of the injection may pass through the release mechanism and treat the patient when the formulation is injected, and a second portion of the formulation can be contained in the chamber when the formulation is injected.

FIG. 6B-1 shows interconnecting channels 156 extending from first side 150S1 to second side 150S2 of the porous structure as in FIG. 6B. The interconnecting channels 156 extend to a plurality of openings 158A comprising a first opening 158A1, a second opening 158A2 and an Nth opening 158AN on the first side 150S1. The interconnecting channels 156 extend to a plurality of openings 158B comprising a first opening 158B1, a second opening 158B2 and an Nth opening 158BN on the second side 150S2. Each of the openings of the plurality of channels on the first side is connected to each of the openings of plurality of channels on the second side, such that effective length traveled along the channels is greater than thickness 150T. The channel parameter can be within a range from about 1.1 to about 10, such that the effective length is within a range from about 1.1 to 10 times the thickness 150T. For example, the channel parameter can be about 1 and the porosity about 0.2, such that the effective length corresponds to at least about 5 times the thickness 150T.

FIG. 6B-2 shows a plurality of paths of the therapeutic agent along the interconnecting channels extending from a first side 150S1 to a second side 150S2 of the porous structure as in FIGS. 6B and 6B-1. The plurality of paths comprises a first path 156P1 extending from the first side to the second side, a second path 156P2 extending from the first side to the second side and a third path 156P3 extending from the first side to the second side, and many additional paths. The effect length of each of first path P1, second path P2 and third path P3 is substantially similar, such that each opening on the first side can release the therapeutic agent to each interconnected opening on the second side. The substantially similar path length can be related to the sintered grains of material and the channels that extend around the sintered material. The porous structure may comprise randomly oriented and connected grains of material, packed beads of material, or combinations thereof. The channel parameter can be related to the structure of the sintered grains of material and corresponding interconnecting channels, porosity of the material, and percolation threshold. Work in relation to embodiments shows that the percolation threshold of the sintered grains may be below the porosity of the porous frit structure, such that the channels are highly inter-connected. The sintered grains of material can provide interconnected channels, and the grains can be selected to provide desired porosity and channel parameters and RRI as described herein.

The channel parameter and effective length from the first side to the second side can be configured in many ways. The channel parameter can be greater than 1 and within a range from about 1.2 to about 5.0, such that the effective length is within a range about 1.2 to 5.0 times the thickness 150T, although the channel parameter may be greater than 5, for example within a range from about 1.2 to 10. For example, the channel parameter can be from about 1.3 to about 2.0, such that the effective length is about 1.3 to 2.0 times the thickness 150T. For example, experimental testing has shown the channel parameter can be from about 1.4 to about 1.8, such that the effective length is about 1.4 to 1.8 times the thickness 150T, for example about 1.6 times the thickness. These values correspond to the paths of the channels around the sintered grains of material, and may correspond, for example, to the paths of channels around packed beads of material.

FIG. 6B-3 shows blockage of the openings with a covering 156B and the plurality of paths of the therapeutic agent along the interconnecting channels extending from a first side to a second side of the porous structure as in FIGS. 6B and 6B-1. A plurality of paths 156PR extend from the first side to the second side couple the first side to the second side where one of the sides is covered, such that the flow rate is maintained when one of the sides is partially covered.

FIG. 6B-4 shows blockage of the openings with particles 156PB and the plurality of paths of the therapeutic agent along the interconnecting channels extending from a first side to a second side of the porous structure as in FIGS. 6B and 6B-1. The plurality of paths 156PR extend from the first side to the second side couple the first side to the second side where one of the sides is covered, such that the flow rate is maintained when one of the sides is partially covered.

FIG. 6B-5 shows an effective cross-sectional size 150DE and area 150EFF corresponding to the plurality of paths of the therapeutic agent along the interconnecting channels extending from a first side to a second side of the porous structure as in FIGS. 6B and 6B-1. The effective cross sectional area of the interconnecting channels corresponds to the internal cross-sectional area of the porous structure disposed between the openings of the first side and the openings of the second side, such that the rate of release can be substantially maintained when the channels are blocked on the first side and the second side.

The rigid porous structure can be shaped and molded in many ways, for example with tubular shapes, conical shapes, discs and hemispherical shapes. The rigid porous structure may comprise a molded rigid porous structure. The molded rigid porous structure may comprise at least one of a disk, a helix or a tube coupled to the reservoir and configured to release the therapeutic agent for the extended period.

FIG. 6C shows a rigid porous structure as in FIG. 6B incorporated into a scleral tack 601 as described in U.S. Pat. No. 5,466,233. The scleral tack comprises a head 602, a central portion 603 and a post 604. The post may comprise the reservoir 605 and the rigid porous structure 606 as described above. The porous structure may comprise a molded conical structure having a sharp tip configured for insertion into the patient. Alternatively or in combination, the tip may be rounded.

FIG. 6E shows a plurality of rigid porous structures as in FIG. 6B incorporated with a drug delivery device for sustained release as described in U.S. Pat. No. 5,972,369. The therapeutic device comprises a reservoir 613 to contain the therapeutic agent and an impermeable and non-porous outer surface 614. The reservoir is coupled to a rigid porous structure 615 that extends to a distal end 617. The rigid porous structure comprises an exposed area 616 on the distal end to release the therapeutic agent, and the impermeable and non-porous outer surface may extend to the distal end.

FIG. 6D shows a rigid porous structure as in FIG. 6B incorporated with a delivery device for sustained release as described in U.S. Pat. Pub. 2003/0014036 A1. The drug delivery device comprises an inlet port 608 on the proximal end and a hollow body 609 coupled to the inlet port. The hollow body comprises many openings 612 that allow a solution injected into the inlet port to pass from the hollow body into a balloon 610. The balloon comprises a distal end 611 disposed opposite the injection port. The balloon comprises a plurality of the rigid porous structures 607, as described above. Each of the plurality of porous rigid structures comprises a first surface exposed to the interior of the balloon and a second surface configured to contact the vitreous. The calculated area can be the combined area of the plurality of porous rigid structures as noted above.

FIG. 6F shows a rigid porous structure as in FIG. 6B incorporated with a non-linear body member 618 for sustained release as described in U.S. Pat. No. 6,719,750. The non-linear member may comprise a helical shape. The non-linear member can be coupled to a cap 619 on the proximal end 620. The non-linear member may comprise a lumen 621 filled with therapeutic agent so as to comprise a reservoir 622. The porous structure 623 can be disposed on a distal end 624 of the non-linear member to release the therapeutic agent. The porous structure may be located at additional or alternative locations of the non-linear member. For example a plurality of porous structures may be disposed along the non-linear member at locations disposed between the cap and distal end so as to release therapeutic agent into the vitreous humor when the cap is positioned against the sclera.

FIG. 6G shows porous nanostructures, in accordance with embodiments. The porous structure 150 may comprise a plurality of elongate nano-channels 156NC extending from a first side 150S1 of the porous structure to a second side 150S2 of the porous structure. The porous structure 150 may comprise a rigid material having the holes formed thereon, and the holes may comprise a maximum dimension across such as a diameter. The diameter of the nano-channels may comprise a dimension across, for example from about 10 nm across, to about 1000 nm across, or larger. The channels may be formed with etching of the material, for example lithographic etching of the material. The channels may comprise substantially straight channels such that the channel parameter F comprises about 1, and the parameters area A, and thickness or length L correspond to the combined cross-sectional area of the channels and the thickness or length of the porous structure.

The porous structure 150 may comprise interconnecting nano-channels, for example formed with a sintered nano-material.

The injection of therapeutic agent into the device 100 as described herein can be performed before implantation into the eye, or alternatively, when the therapeutic device is implanted into the eye.

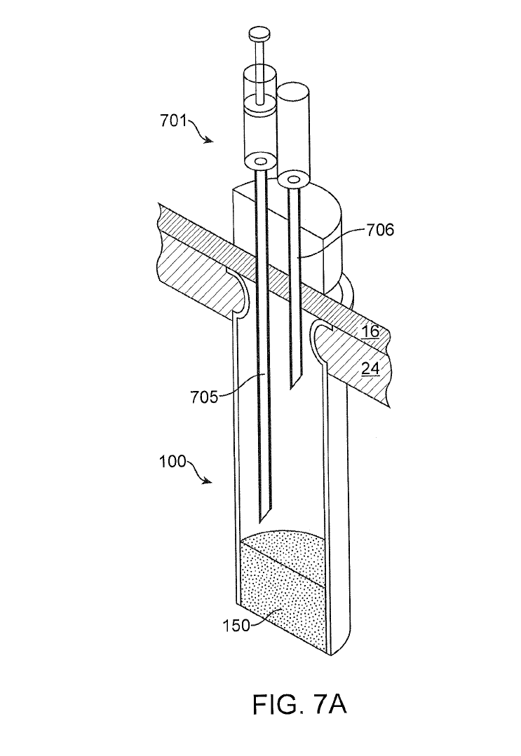

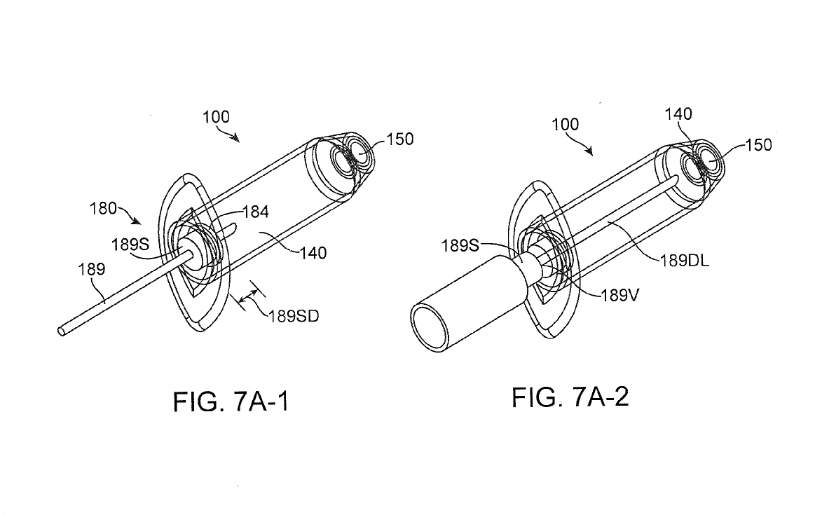

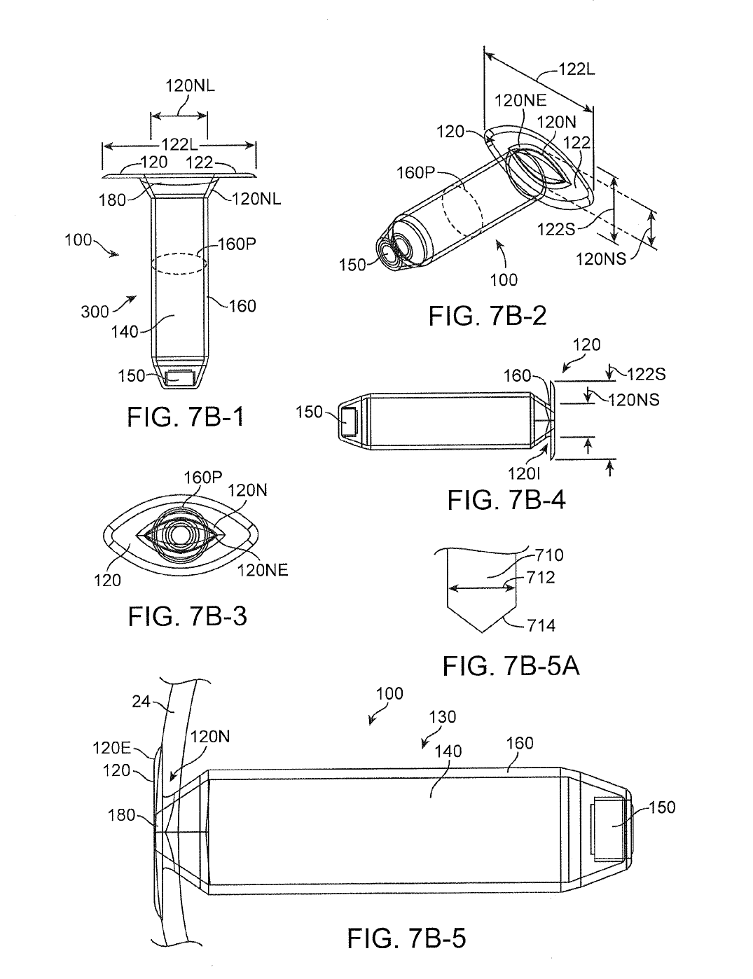

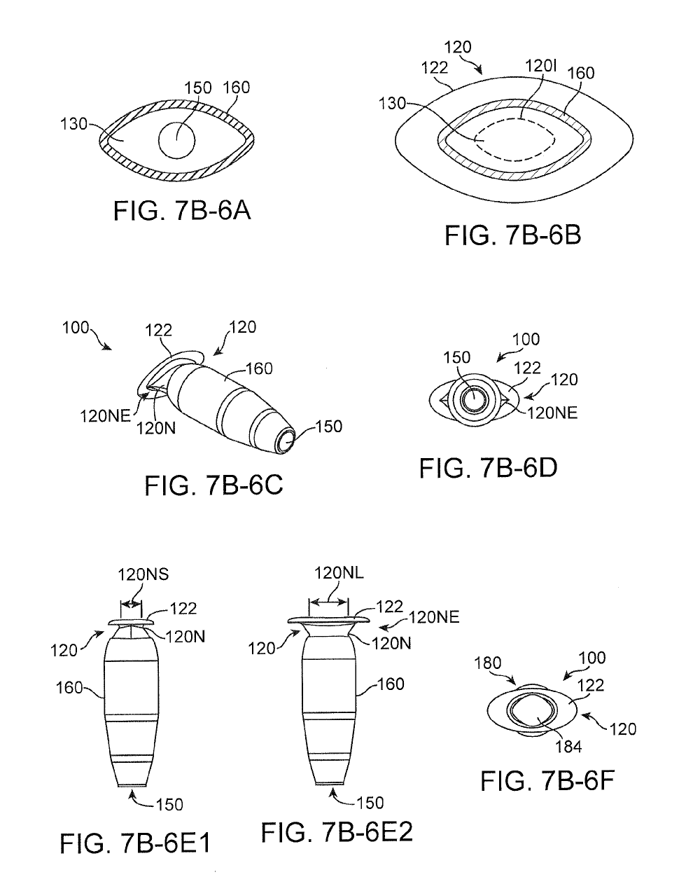

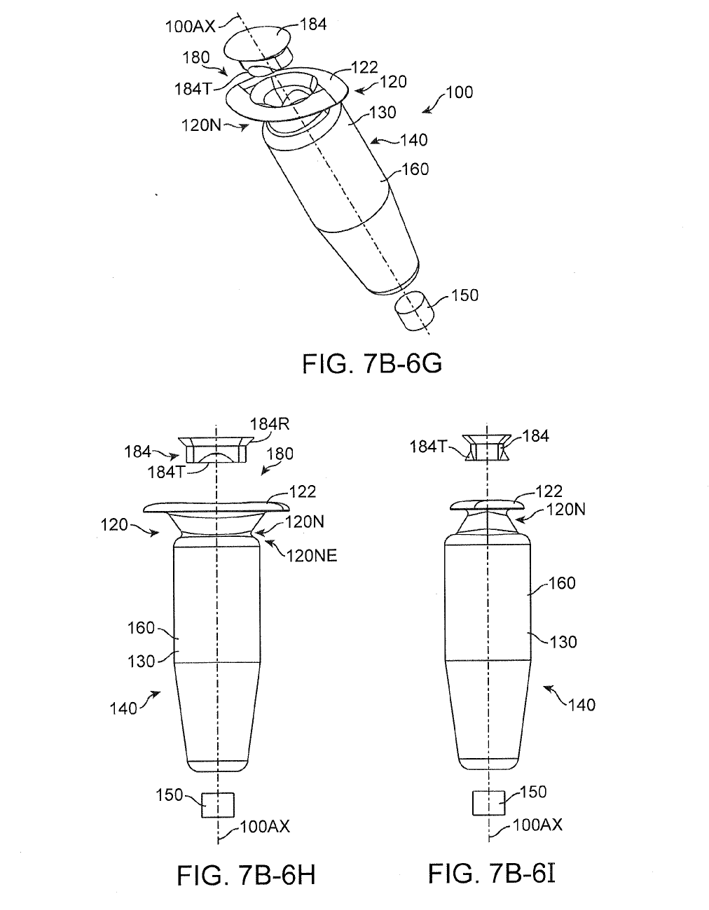

FIG. 7 shows a therapeutic device 100 coupled to an injector 701 that removes material from the device and injects therapeutic agent 702 into the device. The injector picks up spent media 703 and refills the injector with fresh therapeutic agent. The therapeutic agent is injected into the therapeutic device. The spent media is pulled up into the injector. The injector may comprise a stopper mechanism 704.

The injector 701 may comprise a first container 702C to contain a formulation of therapeutic agent 702 and a second container 703C to receive the spent media 703. Work in relation to embodiments suggests that the removal of spent media 703 comprising material from the container reservoir of the therapeutic device can remove particulate from the therapeutic device, for example particles comprised of aggregated therapeutic agent such as protein. The needle 189 may comprise a double lumen needle with a first lumen coupled to the first container and a second lumen coupled to the second container, such that spent media 703 passes from the container reservoir of device 100 to the injector. A valve 703V, for example a vent, can be disposed between the second lumen and the second container. When the valve is open and therapeutic agent is injected, spent media 703 from the container reservoir of the therapeutic device 100 passes to the second container of the injector, such that at least a portion of the spent media within the therapeutic device is exchanged with the formulation. When the valve is closed and the therapeutic agent is injected, a portion of the therapeutic agent passes from the reservoir of the therapeutic device into the eye. For example, a first portion of formulation of therapeutic agent can be injected into therapeutic device 100 when the valve is open such that the first portion of the formulation is exchanged with material disposed within the reservoir; the valve is then closed and a second portion of the formulation is injected into therapeutic device 100 such that at least a portion of the first portion passes through the porous structure into the eye. Alternatively or in combination, a portion of the second portion of injected formulation may pass through the porous structure when the second portion is injected into the eye. The second portion of formulation injected when the valve is closed may correspond to a volume of formulation that passes through the porous structure into the vitreous humor to treat the patient immediately.

The needle 189 may comprise a dual lumen needle, for example as described with reference to FIG. 7A2 shown below.

FIG. 7A shows a therapeutic device 100 coupled to an injector 701 to inject and remove material from the device. The injector may comprise a two needle system configured to insert into a container of the device. The injector may simultaneously inject therapeutic agent through the first needle 705 (the injection needle) while withdrawing liquid from the device through the second needle 706 (the vent needle). The injection needle may be longer and/or have a smaller diameter than the vent needle to facilitate removal of prior material from the device. The vent needle may also be attached to a vacuum to facilitate removal of prior material from the device.

FIG. 7A-1 shows a therapeutic device 100 comprising a penetrable barrier coupled to an injector needle 189 comprising a stop 189S that positions the distal end of the needle near the proximal end of the reservoir 130 of the device to flush the reservoir with ejection of liquid formulation through the porous frit structure, in accordance with embodiments. For example, the injector needle may comprise a single lumen needle having a bevel that extends approximately 0.5 mm along the shaft of the needle from the tip of the needle to the annular portion of the needle. The stop can be sized and positioned along an axis of the needle such that the needle tip extends a stop distance 189SD into the reservoir as defined by the length of the needle from the stop to the tip and the thickness of the penetrable barrier, in which the stop distance is within a range from about 0.5 to about 2 mm. The reservoir may extend along an axis of the therapeutic device distance within a range from about 4 to 8 mm. A volume comprising a quantity of liquid formulation within a range from about 20 to about 200 uL, for example about 50 uL can be injected into the therapeutic device with the needle tip disposed on the distal end. The volume of the reservoir can be less than the injection volume of the formulation of therapeutic agent, such that liquid is flushed through the porous structure 150. For example, the reservoir may comprise a volume within a range from about 20 to 40 uL, and the injection volume of the liquid formulation of therapeutic agent may comprise about 40 to 100 uL, for example about 50 uL.

FIG. 7A-2 shows a therapeutic device comprising a penetrable barrier coupled to a needle 189 of an injector 701 to inject and remove material from the device such that the liquid in the reservoir 130 is exchanged with the injected formulation. The needle comprises at least one lumen and may comprise a concentric double lumen needle 189DL with a distal end coupled to the inner lumen to inject formulation of the therapeutic agent into the therapeutic device and a proximal vent 189V to receive liquid into the needle when the formulation is injected. Alternatively, the vent may correspond to an opening on the distal end of the inner lumen of the needle and the outer lumen may comprise a proximal opening to inject therapeutic agent formulation into a proximal portion of the container reservoir.

Work in relation to the injector embodiments indicates that a filling efficiency of at least about 80%, for example 90% or more can be achieved with injector apparatus and needles as described above.

FIG. 7B-1 shows a side cross-sectional view of therapeutic device 100 comprising a retention structure having a cross-section sized to fit in an elongate incision. The cross-section sized to fit in the elongate incision may comprise a narrow portion 120N of retention structure 120 that is sized smaller than the flange 122. The narrow portion 120N sized to fit in the elongate incision may comprise an elongate cross section 120NE sized to fit in the incision. The narrow portion 120N may comprise a cross-section having a first cross-sectional distance across, or first dimensional width, and a second cross-sectional distance across, or second dimensional width, in which the first cross-sectional distance across is greater than the second cross-sectional distance across such that the narrow portion 120N comprises an elongate cross-sectional profile.

The elongate cross section 120NE of the narrow portion 120N can be sized in many ways to fit the incision. The elongate cross section 120NE comprises a first dimension longer than a second dimension and may comprise one or more of many shapes such as dilated slot, dilated slit, lentoid, oval, ovoid, or elliptical. The dilated slit shape and dilated slot shape may correspond to the shape sclera tissue assumes when cut and dilated. The lentoid shape may correspond to a biconvex lens shape. The elongate cross-section of the narrow portion may comprise a first curve along a first axis and a second curve along a second axis different than the first curve.

Similar to the narrow portion 120N of the retention structure, the container reservoir may comprise a cross-sectional profile

FIG. 7B-2 shows an isometric view of the therapeutic device as in FIG. 7B-1.

FIG. 7B-3 shows a top view of the therapeutic device as in FIG. 7B-1.

FIG. 7B-4 shows a side cross sectional view along the short side of the retention structure of the therapeutic device as in FIG. 7B-1.

FIG. 7B-5 shows a bottom view of the therapeutic device as in FIG. 7B-1 implanted in the sclera.

FIG. 7B-5A shows a cutting tool 710 comprising a blade 714 having a width 712 corresponding to perimeter 160P of the barrier 160 and the perimeter 160NP of the narrow portion. The cutting tool can be sized to the narrow portion 120N so as to seal the incision with the narrow portion when the narrow portion is positioned against the sclera. For example, the width 712 may comprise about one half of the perimeter 160P of the barrier 160 and about one half of the perimeter 160NP of the narrow portion 160N. For example, the outside diameter of the tube of barrier 160 may comprise about 3 mm such that the perimeter of 160P comprises about 6 mm, and the narrow portion perimeter 160NP may comprise about 6 mm. The width 712 of the blade 714 may comprise about 3 mm such that the incision comprises an opening having a perimeter of about 6 mm so as to seal the incision with the narrow portion 160NP. Alternatively, perimeter 160P of barrier 160 may comprise a size slightly larger than the incision and the perimeter of the narrow portion 106NP.

The retention structure comprises a narrow portion 120N having a short distance 120NS and a long distance 120NL so as to fit in an elongate incision along the pars plana of the eye. The retention structure comprises an extension 122. The extension of the retention structure 120E comprises a short distance across 122S and a long distance across 122L, aligned with the short distance 122NS and the long distance 122NL of the narrow portion 120N of the retention structure 120. The narrow portion 120N may comprise an indentation 1201 sized to receive the sclera.

FIGS. 7B-6A and 7B-6B show distal cross-sectional view and a proximal cross-sectional view, respectively, of therapeutic device 100 comprising a non-circular cross-sectional size. The porous structure 150 can be located on a distal end portion of the therapeutic device, and the retention structure 120 can be located on a proximal portion of therapeutic device 100. The barrier 160 defines a size of reservoir 130. The barrier 160 and reservoir 130 may each comprise an elliptical or oval cross-sectional size, for example. The barrier 160 comprises a first cross-sectional distance across reservoir 130, and a second cross-sectional distance across reservoir 130, and the first distance across may extend across a long (major) axis of an ellipse and the second distance across may extend across a short (minor) axis of the ellipse. This elongation of the device along one direction can allow for increased drug in the reservoir with a decrease interference in vision, for example, as the major axis of the ellipse can be aligned substantially with the circumference of the pars plana region of the eye extending substantially around the cornea of the eye, and the minor axis of the ellipse can be aligned radially with the eye so as to decrease interference with vision as the short axis of the ellipse extends toward the optical axis of the eye corresponding to the patient's line of sight through the pupil. Although reference is made to an elliptical or oval cross-section, many cross-sectional sizes and shapes can be used such as rectangular with a short dimension extending toward the pupil of the eye and the long dimension extending along the pars plana of the eye.

The retention structure 120 may comprise structures corresponding to structure of the cross-sectional area. For example, the extension 122 may comprise a first distance across and a second distance across, with the first distance across greater than the second distance across. The extension may comprise many shapes, such as rectangular, oval, or elliptical, and the long distance across can correspond to the long distance of the reservoir and barrier. The retention structure 120 may comprise the narrow portion 120N having an indentation 1201 extending around an access port to the therapeutic device, as described above. The indentation 1201 and extension 122 may each comprise an elliptical or oval profile with a first long (major) axis of the ellipse extending in the first direction and a second short (minor) axis of the ellipse extending in the second direction. The long axis can be aligned so as to extend circumferentially along the pars plana of the eye, and the short axis can be aligned so as to extend toward the pupil of the eye, such that the orientation of device 100 can be determined with visual examination by the treating physician.

FIG. 7B-6C shows an isometric view of the therapeutic device having a retention structure comprising a narrow portion 120N with an elongate cross-sectional size 120NE.

FIG. 7B-6D shows a distal end view of the therapeutic device as in FIG. 7B-6C.

FIG. 7B-6E1 shows a side view of the short distance 120NS of the narrow portion 120N of the therapeutic device as in FIG. 7B-6C.

FIG. 7B-6E2 shows a side view of the long distance 120NL of the narrow portion 120N of the therapeutic device 100 as in FIG. 7B-6C.

FIG. 7B-6F shows a proximal view of the therapeutic device as in FIG. 7B-6C.

FIG. 7B-6G to FIG. 7B-6I show exploded assembly drawings for the therapeutic device 100 as in FIGS. 7B-6C to 7B-6F. The assembly drawings of FIGS. 7B-6G, FIG. 7B-6H and FIG. 7B-6I show isometric and thin side profiles views, respectively, of the elongate portion 120NE of the narrow portion of the retention structure 120N. The therapeutic device 100 has an elonagate axis 100AX.

The penetrable barrier 184, for example the septum, can be inserted into the acess port 180. The penetrable barrier may comprise an elastic material sized such that the penetrable barrier can be inserted into the access port 180. The penetrable barrier may comprise one or more elastic materials such as siloxane or rubber. The penetrable barrier may comprise tabs 184T to retain the penetrable barrier in the acces port. The penetrable barrier 184 may comprise a beveled upper rim 184R sized to seal the access port 180. The access port 180 of the reservoir container 130 may comprise a beveled upper surface to engage the beveled rim and seal the penetrable barrier against the access port 180 when the tabs 184T engage an inner annular or elongate channel of the access port. The penetrable barrier 184 may comprise an opaque material, for example a grey material, for example silicone, such that the penetrable barrier can be visualized by the patient and treating physician.

The reservoir container 130 of the device may comprise a rigid biocompatible material that extends at least from the retention structure to the rigid porous structure, such that the reservoir comprises a substantially constant volume when the therapeutic agent is released with the rigid porous structure so as to maintain a stable release rate profile, for example when the patient moves. Alternatively or in combination, the reservoir container 130 may comprise an optically transmissive material such that the reservoir container 130 can be translucent, for example transparent, such that the chamber of reservoir 140 can be visualized when the device is loaded with therapeutic agent outside the patient prior to implantation, for example when injected with a formulation of therapeutic agent prior to implantation in the physcian's office. This visualization of the reservoir 140 can be helpful to ensure that the reservoir 140 is properly filled with therapeutic agent by the treating physician or assistant prior to implantation. The reservoir container may comprise one or more of many biocomaptible materials such as acrylates, polymethylmethacrylate, siloxanes, metals, titanium stainless steel, polycarbonate, polyetheretherketone (PEEK), polyethylene, polyethylene terephthalate (PET), polyimide, polyamide-imide, polypropylene, polysulfone, polyurethane, polyvinylidene fluoride or PTFE. The biocompatible material of the reservoir container may comprise an optically transmissive material such as one or more of acrylate, polyacrylate, methlymethacraylate, polymethlymethacrylate (PMMA), polyacarbonate or siloxane. The reservoir container 130 can be machined from a piece of material, or injection molded, so as to form the retention structure 120 comprising flange 122 and the elongate narrow portion 120NE. The flange 122 may comprise a translucent material such that the physician can visualize tissue under the flange to assess the patient and to decrease appearance of the device 100 when implanted. The reservoir container 130 may comprise a channel extending along axis 100AX from the access port 180 to porous structure 150, such that formulation injected into device 100 can be released in accordance with the volume of the reservoir and release rate of the porous structure 150 as described herein. The porous structure 150 can be affixed to the distal end of therapeutic device 100, for example with glue. Alternatively or in combination, the distal end of the reservoir container 130 may comprise an inner diameter sized to receive the porous structure 150, and the reservoir container 130 may comprise a stop to position the porous structure 150 at a predetermined location on the distal end so as to define a predetermined size of reservoir 140.

FIG. 7C-1 shows an expandable therapeutic device 790 comprising expandable barrier material 160 and support 160S in an expanded configuration for extended release of the therapeutic agent. The expanded configuration can store an increased amount of therapeutic agent, for example from about 30 uL to about 100 uL. The expandable device comprises a retention structure 120, an expandable reservoir 140. The support 160S may comprise a resilient material configured for compression, for example resilient metal or thermoplastic. Alternatively, the expandable support may be bent when expanded. The expandable device comprises the porous structure 150 disposed on a distal end, and affixed to the expandable support. The expandable device may comprise an access port 180, for example with a penetrable barrier 184. In the expanded configuration, the device is substantially clear from a majority of the optical path OP of the patient

The support 160S of the barrier 160 can provide a substantially constant volume of the reservoir in the expanded configuration. The substantially constant volume, for example +/−25%, can be combined with the release rate index of the porous structure 150 so as to tune the expanded reservoir and porous structure to the volume of therapeutic agent to be injected into the therapeutic device as described herein. The barrier 160 may comprise a thin compliant material, for example a membrane, and the support 160S can urge the barrier 160 to an expanded configuration so as to define the reservoir chamber having the substantially constant volume.

Tuning of Therapeutic Device for Sustained Release Based on an Injection of a Formulation

The properties of the porous structures as described herein are suitable for use with therapeutic devices so as to tune the release of therapeutic agent.

The therapeutic device 100 can be tuned to deliver a target therapeutic concentration profile based on the volume of formulation injected into the device. The injected volume may comprise a substantially fixed volume, for example within about +/−30% of an intended pre-determined target volume. The volume of the reservoir can be sized with the release rate index so as to release the therapeutic agent for an extended time substantially greater than the treatment time of a corresponding bolus injection. The device can also be tuned to release the therapeutic agent based on the half-life of the therapeutic agent in the eye. The device volume and release rate index comprise parameters that can be tuned together based on the volume of formulation injected and the half-life of the therapeutic agent in the eye. The following equations can be used to determine therapeutic device parameters suitable for tuning the device.

Rate=Vr(dCr/dt)=−D(PA/TL)Cr
where Rate=Rate of release of therapeutic agent from device
Cr=concentration of therapeutic agent in reservoir
Vr=volume of reservoir
D=Diffusion constant

PA/TL=RRI
P=porosity
A=area
T=tortuosity=F=channel parameter.
For a substantially fixed volume injection,

Cr0=(Injection Volume)(Concentration of Formulation)/Vr
Where Cr0=initial concentration in reservoir following injection of formulation For Injection Volume=50 uL

Cr0=(0.05 mL)(10 mg/mL)/Vr(1000 ug/1 mg)=500 ug/Vr

Rate=x(500 ug)exp(−xt)
where t=time

x=(D/Vr)(PA/TL)
With a mass balance on the vitreous

Vv(dCv/dt)=Rate from device=kVvCv
where Vv=volume of vitreous (about 4.5 ml)
Cv=concentration of therapeutic agent in vitreous
k=rate of drug from vitreous (proportional to 1/half-life of drug in vitreous)
For the situation appropriate for the embodiments as described herein where Cv remains substantially constant and changes slowly with time (i.e., dCv/dt is approximately 0),

Cv=(Rate from device)/(kVv)

Since kVv is substantially constant, the max value of Cv will correspond to conditions that maximize the Rate from the device. At a given time since injection into the device (e.g., 180 days), the maximum Cv is found at the value of x that provides the maximum rate. The optimal value of x satisfies

d(Rate)/dx=0 at a given time.

Rate=500(x)exp(−xt)=f(x)g(x)where f(x)=500x and g(x)=exp(−xt)

d(Rate)/dx=f′(x)g(x)+f(x)g′(x)=500(1−xt)exp(−xt)
For a given time, t, d(Rate)/dx=0 when 1−xt=0 and xt=1
The rate is maximum when (D/Vr)(PA/TL)t=1.
For a given volume, optimal PA/TL=optimal RRI=Vr/(Dt)
Therefore, the highest Cv at a given time, t, occurs for the optimal RRI=(PA/FL) for a given Vr.
Also, the ratio (Vr)/(RRI)=(Vr)/(PA/TL)=Dt will determine the optimal rate at the time.

The above equations provide approximate optimized values that, when combined with numerical simulations, can provide optimal values of Vr and PA/TL. The final optimum value can depend on additional parameters, such as the filling efficiency.

The above parameters can be used to determine the optimal RRI, and the therapeutic device can be tuned to the volume of formulation injected into the device with a device reservoir volume and release rate index within about +/−50% of the optimal values, for example +/−30% of the optimal values. For example, for an optimal release rate index of the porous structure and an optimal reservoir volume sized to receive a predetermined quantity of therapeutic agent, e.g., 50 uL, so as to achieve therapeutic concentrations above a minimum inhibitory concentration for a predetermined extended time such as 90 days, the maximum volume of the reservoir can be limited to no more than about twice the optimal volume. This tuning of the reservoir volume and the porous structure to the injected volume of the commercially available formulation can increase the time of release of therapeutic amounts from the device as compared to a much larger reservoir volume that receives the same volume of commercially available injectable formulation. Although many examples as described herein show a porous frit structure and reservoir volume tuned together to receive a quantity of formulation and provide release for an extended time, the porous structure tuned with the reservoir may comprise one or more of a porous frit, a permeable membrane, a semi-permeable membrane, a capillary tube or a tortuous channel, nano-structures, nano-channels or sintered nano-particles, and a person of ordinary skill in the art can determine the release rate characteristics, for example a release rate index, so as to tune the one or more porous structures and the volume to receive the quantity of the formulation and release therapeutic amounts for an extended time.

As an example, the optimal RRI at 180 days can be determined for a reservoir volume of about 125 uL. Based on the above equations (Vr/Dt)=optimal RRI, such that the optimal RRI at 180 days is about 0.085 for the 50 uL formulation volume injected into the device. The corresponding Cv is about 3.19 ug/mL at 180 days based on the Rate of drug released from the device at 180 days and the rate of the drug from the vitreous (k corresponding to a half-life of about 9 days). A device with a container reservoir volume of 63 uL and RRI of 0.044 will also provide the optimal Cv at 180 days since the ratio of Vr to PA/TL is also optimal. Although an optimal value can be determined, the therapeutic device can be tuned to provide therapeutic amounts of drug at a targeted time, for example 180 days, with many values of the reservoir volume and many values of the release rate index near the optimal values, for example within about +/−50% of the optimal values. Although the volume of the reservoir can be substantially fixed, the volume of the reservoir can vary, for example within about +/−50% as with an expandable reservoir such as a balloon reservoir.

The half-life of the drug in the vitreous humor of the eye can be determined based on the therapeutic agent and the type of eye, for example human, rabbit or monkey, such that the half-life may be determined based on the species of the eye, for example. With at least some animal models the half-life of the therapeutic agent in the vitreous humor can be shorter than for human eyes, for example by a factor of about two in at least some instances. For example, the half-life of the therapeutic agent Lucentis™ (ranibizumab) can be about nine days in the human eye and about two to four days in the rabbit and monkey animal models. For small molecules, the half-life in the vitreous humor of the human eye can be about two to three hours and can be about one hour in the monkey and rabbit animal models. The therapeutic device can be tuned to receive the volume of formulation based on the half-life of the therapeutic agent in the human vitreous humor, or an animal vitreous humor, or combinations thereof. Based on the teachings described herein, a person of ordinary skill in the art can determine empirically the half-life of the therapeutic agent in the eye based on the type of eye and the therapeutic agent, such that the revervoir and porous structure can be tuned together so as to receive the volume of formulation and provide therapeutic amounts for the extended time.

### EXPERIMENTAL

### EXAMPLE 1

### Scanning Electron Micrographs of Porous Frit Structures

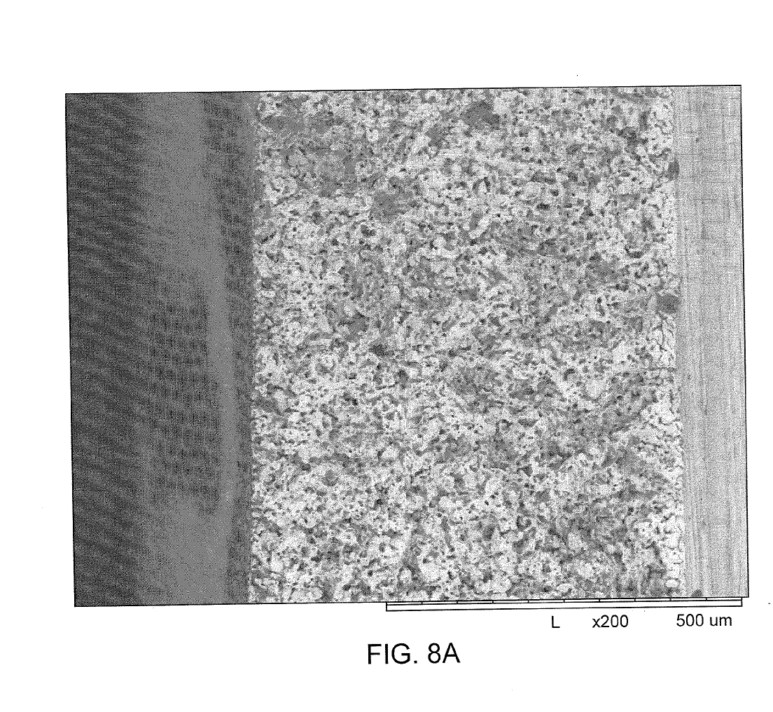

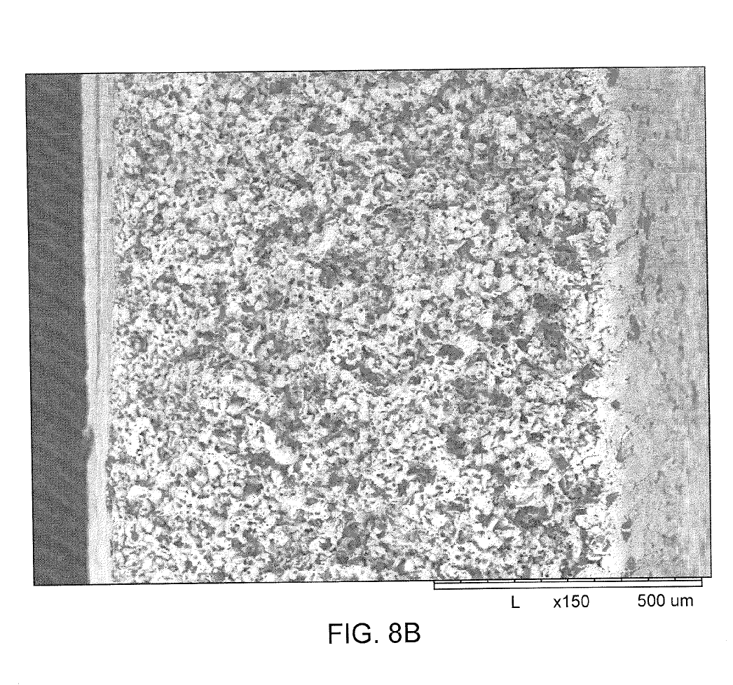

FIGS. 8A and 8B show scanning electron microscope images from fractured edges of porous frit structures of 0.2 media grade and 0.5 media grade porous material, respectively. The commercially available samples were obtained from Mott Corporation and comprised 316L stainless steel. The samples were mechanically fractured so as to show the porous structure and interconnecting channels within the material to release the therapeutic agent. The micrograph images show a plurality of interconnecting channels disposed between openings of the first surface and openings of the second surface.

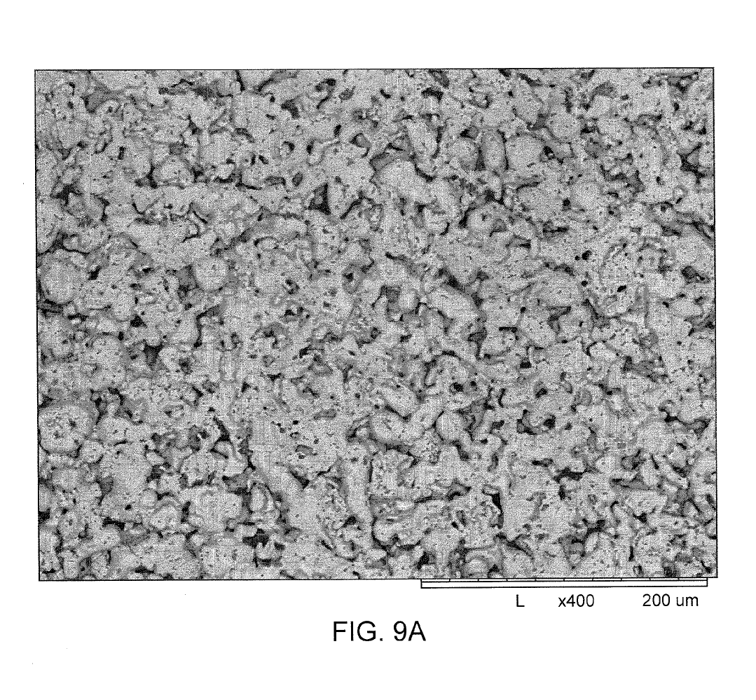

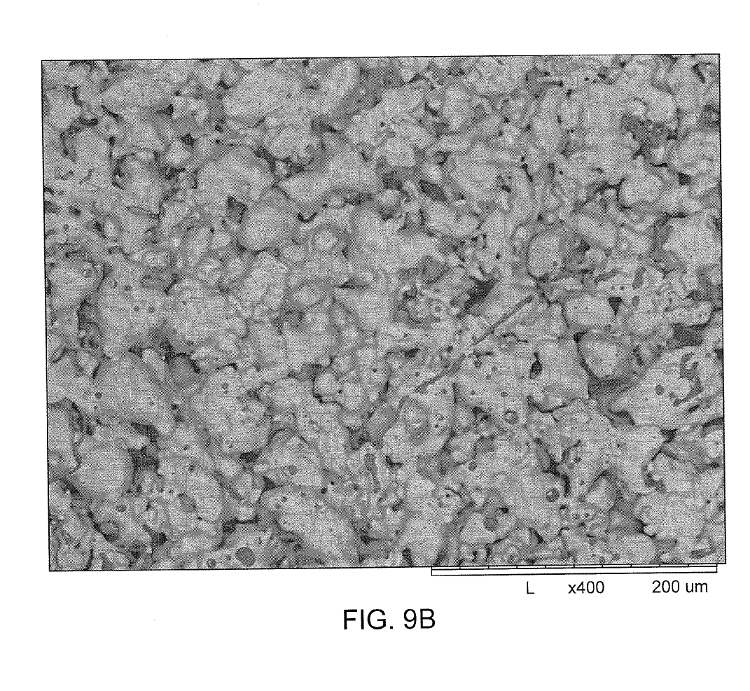

FIGS. 9A and 9B show scanning electron microscope images from surfaces of porous frit structures of media grade of 0.2 and 0.5, respectively, from the samples of FIGS. 8A and 8B. The images show a plurality of openings on the surface connected with interconnecting channels as in FIGS. 8A and 8B.

### EXAMPLE 2

### Porous Frit Structure Mechanical Flow Testing to Identify Porous Frit Structures Suitable for Use with Therapeutic Agent Delivery Devices

The relative characteristics of sample elements can be determined by subjecting the frit to a number of mechanical tests, including but not limited to pressure decay and flow. These tests can be combined with drug release rate information, for example the RRI, so as to determine the release profile of the devices. These tests can be used with the porous structure positioned on the therapeutic device, so as to quantify flow through the porous structure of the device and determine suitable of the porous structure. Similar tests can be used to quantify the porous structure prior to mounting on the therapeutic device. At least some of the therapeutic devices can be evaluated with the gas flow of the porous structure mounted on a partially assembled therapeutic device, for example as a quality control check. In some embodiments, the flow test can be performed on the partially assembled or substantially assembled therapeutic device prior to insertion of the therapeutic agent into the reservoir and prior to insertion into the patient, so as to ensure that the porous structure is suitable for release of the therapeutic agent and affixed to the device, for example a support of the therapeutic device.

These tests may utilize a variety of working fluids, but will most likely use a readily available gas such as air, helium, or nitrogen. To date, flow and pressure decay tests have been used to identify different frit characteristics that may be correlated to other test results such as chemical or pharmacologic performance.

### Fixturing

Each of the test methods above may use a mechanical connection of the test specimen to the test hardware and a number of techniques have been explored and employed. These fixtures include both a means of reliably securing the specimen (such as heat recoverable tubing, elastic tubing, press fits into relatively rigid components, etc.) and a means of coupling (such as a luer, barbed fitting, quick connect coupling, etc.) that allow convenient and repeatable attachment to the test hardware.

### Test Hardware

Each of the desired tests can be developed using commercially available solutions, or by assembling readily available instrumentation to create a custom test arrangement. Again, both of these approaches have been evaluated. A working system will consist of a means for connecting a test specimen, a controllable source (usually, but not limited to pressure), a manometer (or other pressure measurement device), and one or more transducers (pressure, flow, etc.) used to measure the test conditions and/or gather data for further analysis.

### EXAMPLE 2A

### Pressure Decay Test to Identify Porous Structures Suitable for Use with Therapeutic Drug Delivery Devices

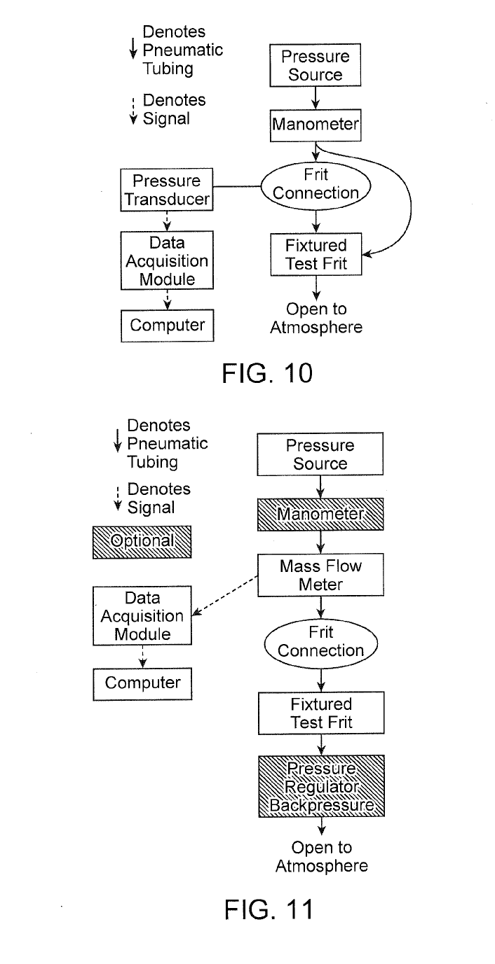

FIG. 10 shows a pressure decay test and test apparatus for use with a porous structure so as to identify porous frit structures suitable for use with therapeutic devices in accordance with embodiments described herein.

One method of pressure decay testing is performed with the hardware shown schematically in FIG. 10. An initial pressure is applied to the system by an outside source such as a syringe, compressed air, compressed nitrogen, etc. The manometer may be configured to display simply the source gage pressure, or the actual differential pressure across the specimen. One side of the fixtured specimen is normally open to atmosphere, creating a pressure which will decay at a rate determined by the properties of the frit being tested. The instantaneous pressure may be measured by a pressure transducer that converts and supplies a signal to a data acquisition module (DAQ) that transfers data to a computer. The rate of pressure drop is then recorded and can be used for comparison to the performance of other frits or an acceptability requirement/specification. This comparison may be made by grossly comparing the pressure at a given time, or by directly comparing the output pressure decay curves.

An example test procedure would pressurize the system to slightly greater than 400 mmHg as displayed by the manometer. The computer and DAQ are configured to begin data acquisition as the pressure drops below 400 mmHg, and a data point is taken approximately every 0.109 seconds. While the test can be stopped at any time, it is likely that standard discreet points along the course of pressure decay data would be selected so as to allow direct comparison of frit flow performance (e.g., time for decay from 400 mmHg to 300 mmHg, and from 400 mmHg to 200 mmHg.)

### EXAMPLE 2B

### Pressure Decay Test to Identify Porous Structures Suitable for Use with Therapeutic Drug Delivery Devices

FIG. 11 shows a pressure flow test and test apparatus suitable for use with a porous structure so as to identify porous frit structures suitable for use with therapeutic devices in accordance with embodiments described herein.

Using a similar hardware set-up, flow through the test specimen can also be characterized. In this test, the source pressure is constantly regulated to a known pressure and the flow of a working fluid is allowed to flow through a mass flow meter and then through the fixtured test frit. As in the pressure decay test, the specific characteristics of the frit determine that rate at which the working fluid will flow through the system. For additional accuracy, pressure at the otherwise open end of the fixture test frit may be regulated to control the back pressure, and therefore, the pressure drop across the specimen.

Flow testing may have advantages over pressure decay testing due to the instantaneous nature of the method. Rather than waiting for the pressure to drop, the flow through a sample should stabilize quickly enabling testing of large number of samples to be performed in rapid fashion.

In an example test procedure, a regulated compressed cylinder would supply the system with a constant source pressure of 30 psig and a constant back pressure of 1 psig. The test fluid would flow through the test frit at a characteristic rate (which is dependent on the pressure, but is expected to be in the 10-500 sccm range) as measured by the mass flow meter.

### EXAMPLE 2C

### Determination of Therapeutic Release Rate Based on Gas Flow

Table 2 shows a table that can be used to determine release of therapeutic agent, for example the RRI, based on the flow of a gas such as oxygen or nitrogen through the porous structure. The flow through the porous structure can be measured with a decay time of the gas pressure, for with the flow rate across the porous structure with a pressure drop across the porous frit structure, as described herein. The flow rate and RRI can be determined based on the media grade of the material, for example as commercially available media grade material available from Mott Corporation. The therapeutic agent can be measured through the porous structure, or a similar test molecule. The initial measurements measured the RRI for Avastin™ with the porous frit structures shown. Based on the teachings described herein, a person of ordinary skill in the art can conduct experiments to determine empirically the correspondence of flow rate with a gas to the release rate of the therapeutic agent.

TABLE 2

| Media | | Length | | | 300 | 200 |
|---|---|---|---|---|---|---|
| Grade | O.D. (in.) | (in.) | RRI | Flow | Decay | Decay |
| 0.2 | 0.031 | 0.049 | 0.019 | | 106 | 256 |
| 0.2 | 0.038 | 0.029 | 0.034 | | | |
| 0.1 | 0.038 | 0.029 | 0.014 | | 81 | 201 |
| 0.2 | 0.038 | 0.029 | 0.033 | | 31 | 78 |

The above partially populated table shows the amount and nature of frit data that can be collected. It is contemplated to use some form of non-destructive testing (i.e., not drug release testing) so as to enable:

a) QC receiving inspection testing of frits

b) QC final device assembly testing

One of ordinary skill in the art can demonstrate a correlation between one or more “flow” tests and the actual drug release testing which relies on diffusion rather than forced gas flow. The data suggests that flow testing of frits can be both repeatable and falls in line with expectations.

Preliminary testing also indicates that the test for the frit alone can be substantially similar to the frit as an assembled device.

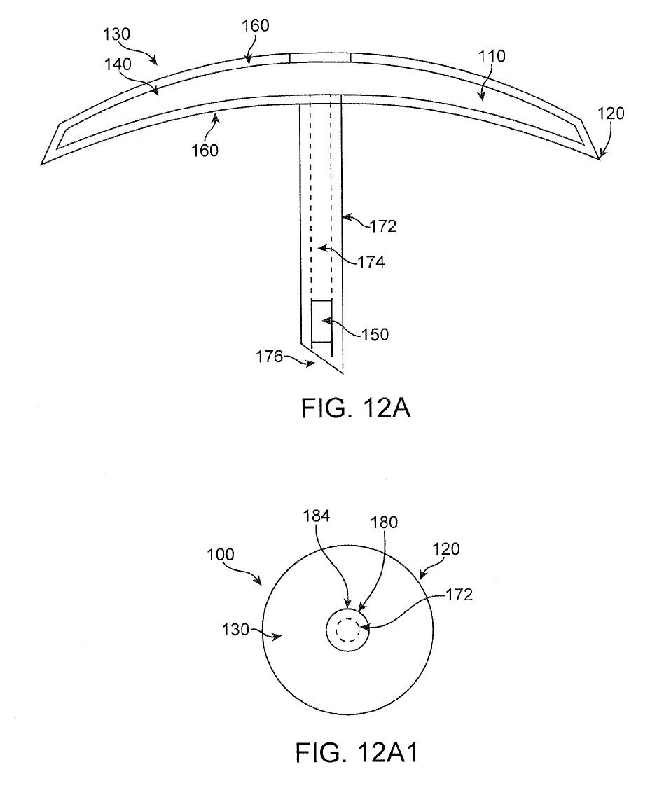

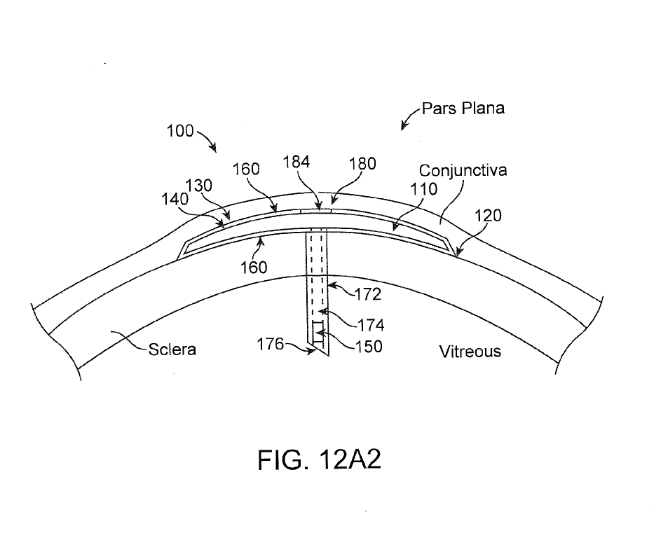

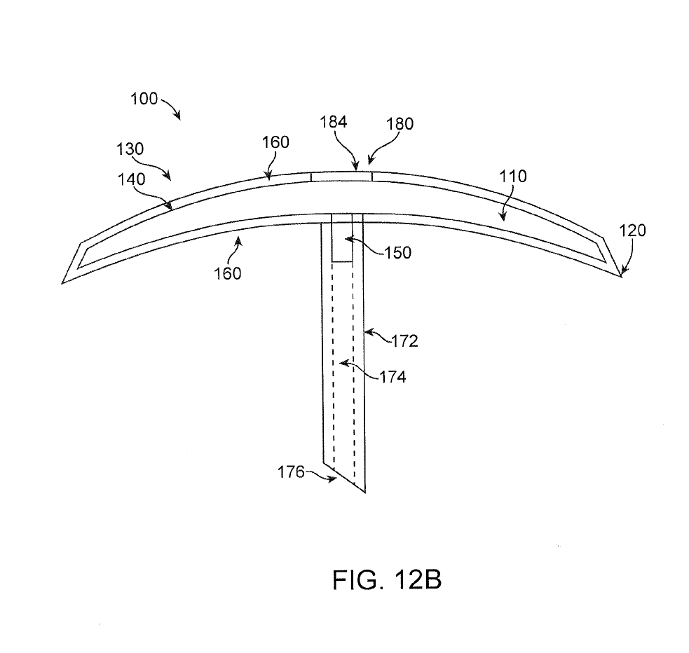

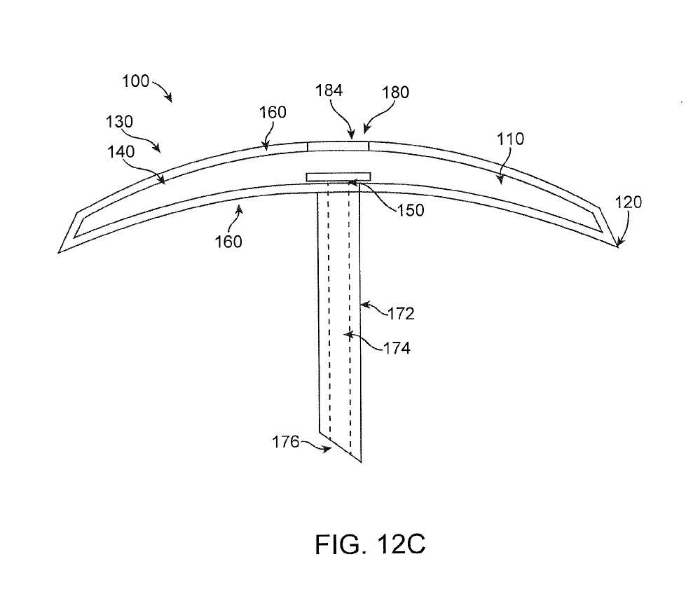

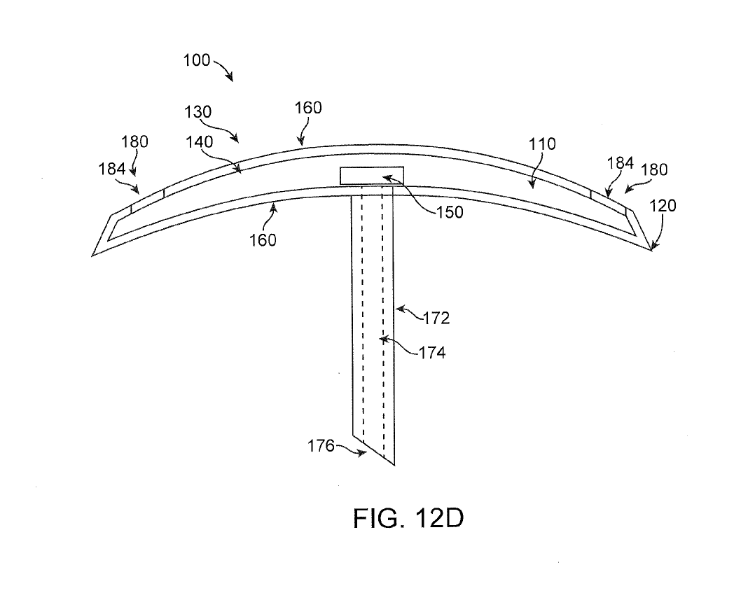

FIGS. 12A and 12A1 show a side cross sectional view and a top view, respectively, of therapeutic device 100 for placement substantially between the conjunctiva and the sclera. The therapeutic agent 110 as described herein can be injected when device 100 is implanted. The therapeutic device 100 comprises container 130 as described herein having penetrable barrier 184 as described herein disposed on an upper surface for placement against the conjunctiva. An elongate structure 172 is coupled to container 130. Elongate structure 172 comprises a channel 174 extending from a first opening coupled to the chamber of the container to a second opening 176 on a distal end of the elongate structure. The porous structure 150 as described herein is located on the elongate structure 172 and coupled to the container 130 so as to release therapeutic agent for an extended period, and a retention structure 120 comprising an extension protruding outward from the container 130 to couple to the sclera and the conjunctiva. The container may comprise barrier 160 as described herein that defines at least a portion of the reservoir, and the container may comprise a width, for example a diameter. The barrier 160 may comprise a rigid material, for example rigid silicone or rigid rubber, or other material as described herein, such that the volume of the chamber of container 130 comprises a substantially constant volume as described herein. Alternatively or in combination, barrier 160 may comprise a soft material, for example when the chamber size is decreased such that the volume can be substantially constant with the decreased chamber size. A soft barrier material can be combined with a rigid material, for example a support material. The diameter can be sized within a range, for example within a range from about 1 to about 8 mm, for example within a range from about 2 to 6 mm and can be about 3 mm, for example.

The container may be coupled to elongate structure 172, and the elongate structure having a length sized so as to extend from the conjunctiva to the vitreous to release the therapeutic agent into the vitreous. The length can be sized within a range, for example within a range from about 2 to about 14 mm, for example within a range from about 4 to 10 mm and can be about 7 mm, for example. The penetrable barrier may comprise a septum disposed on a proximal end of the container, in which the septum comprises a barrier that can be penetrated with a sharp object such as a needle for injection of the therapeutic agent. The porous structure may comprise a cross sectional area sized to release the therapeutic agent for the extended period. The elongate structure 172 can be located near a center of the container 130, or may be eccentric to the center.

The elongate structure 172 can be inserted into the sclera at the pars plana region as described herein.

The barrier 160 can have a shape profile for placement between the conjunctiva and sclera. The lower surface can be shaped to contact the sclera and may comprise a concave shape such as a concave spherical or toric surface. The upper surface can be shaped to contact the conjunctivae and may comprise a convex shape such as a convex spherical or toric surface. The barrier 160 may comprise an oval, an elliptical, or a circular shape when implanted and viewed from above, and the elongate structure 172 can be centered or eccentric to the ellipse. When implanted the long dimension of the oval can be aligned so as to extend along a circumference of the pars plana.

The cross sectional diameter of the elongate structure 172 can be sized to decrease the invasiveness of device 100, and may comprise a diameter of no more than about 1 mm, for example no more than about 0.5 mm, for example no more than about 0.25 mm such that the penetrated sclera seals substantially when elongate structure 172 is removed and the eye can seal itself upon removal of elongate structure 172. The elongate structure 172 may comprise a needle, and channel 174 may comprise a lumen of the needle, for example a 30 gauge needle.

The porous structure 150 may comprise a first side described herein coupled to the reservoir and a second side to couple to the vitreous. The first side may comprise a first area 150 as described herein and the second side may comprise a second area. The porous structure may comprise a thickness as described herein. The porous structure many comprise a diameter. The porous structure may comprise a release rate index, and the chamber of container 130 that defines the volume of reservoir 140 can be sized such that the porous structure and the volume are tuned to receive an amount of therapeutic agent injected with a volume of formulation of therapeutic agent and tuned to release therapeutic amounts for an extended time. Many release rate mechanisms as described herein can be used to tune the release rate and volume to the quantity of therapeutic agent injected as described herein.

The volume of the reservoir 140 defined by the chamber of the container may comprise from about 5 uL to about 2000 uL of therapeutic agent, or for example from about 10 uL to about 200 uL of therapeutic agent.

The porous structure may comprise a needle stop that limits penetration of the needle. The porous structure may comprise a plurality of channels configured for the extended release of the therapeutic agent. The porous structure may comprise a rigid sintered material having characteristics suitable for the sustained release of the material.

FIG. 12A2 shows the therapeutic device 100 implanted with the reservoir between the conjunctiva and the sclera, such that elongate structure 172 extends through the sclera to couple the reservoir chamber to the vitreous humor. When implanted, the porous structure 150 can be located in the vitreous humor, or located between the conjunctiva and sclera, or may extend through the sclera, or combinations thereof.

FIG. 12B shows the porous structure 150 of therapeutic device 100 located in channel 174 near the opening to the chamber of the container 130. The porous structure can extend substantially along the length of elongate structure 172.

FIG. 12C shows the porous structure 150 located within the chamber of container 150 and coupled to the first opening of the elongate structure 172 so as to provide the release rate profile. The porous structure can cover the opening of elongate structure 172 such that therapeutic amounts are released for the extended time as described herein.

FIG. 12D shows a plurality of injection ports spaced apart so as to inject and exchange the liquid of chamber of the container 130 and inject the therapeutic agent into the reservoir chamber of the container 130. The penetrable barrier 184 may comprise a first penetrable barrier located in a first access port formed in the barrier 160 and a second penetrable barrier located in a second access port formed in the barrier 160, and the first barrier can be separated from the second barrier by at least about 1 mm.

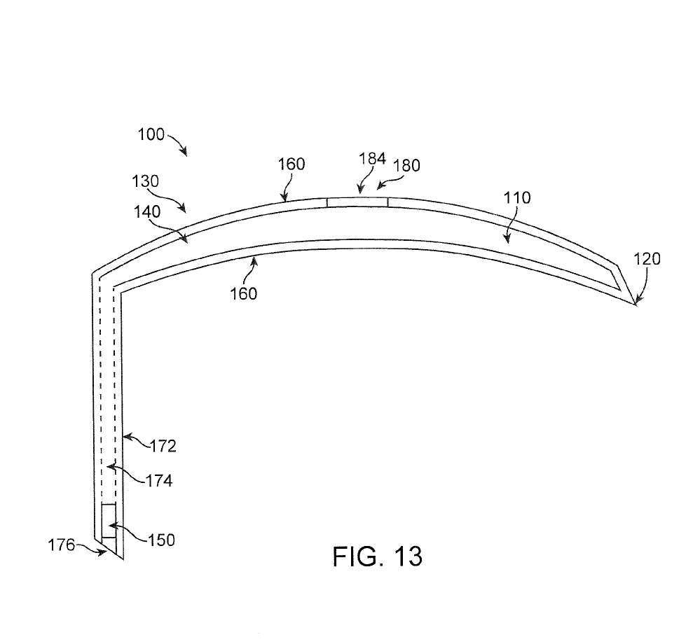

FIG. 13 shows the elongate structure 172 coupled to the container 130 away from the center of container 130 and located near an end of the container.

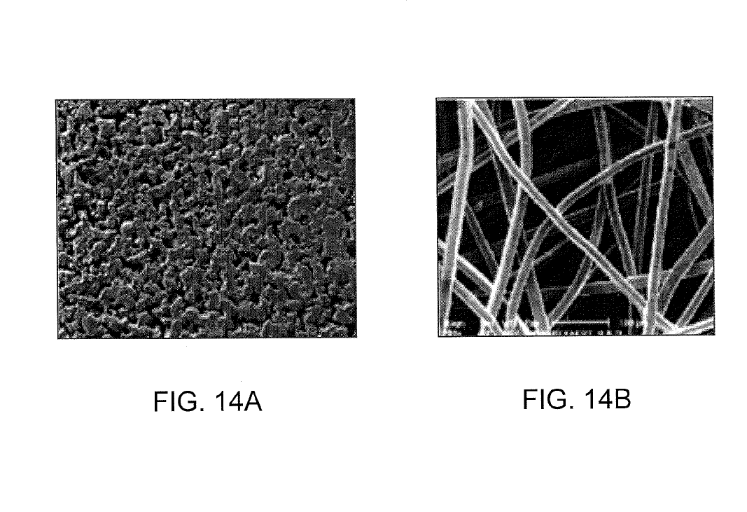

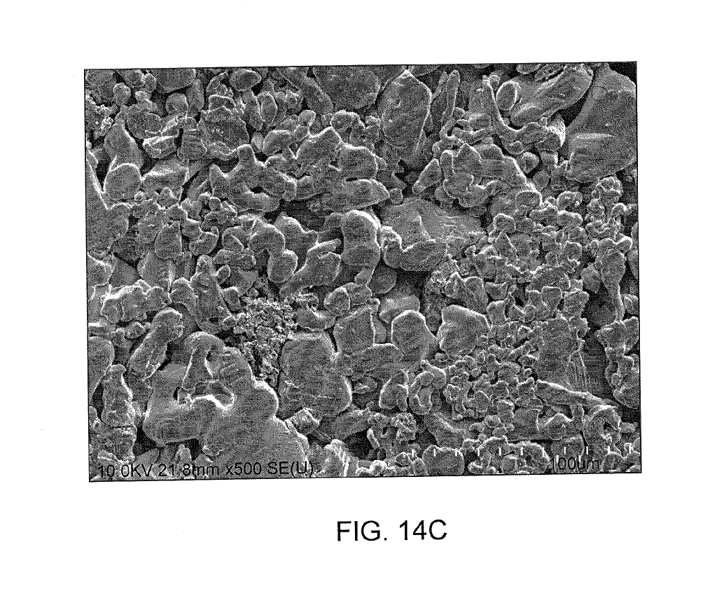

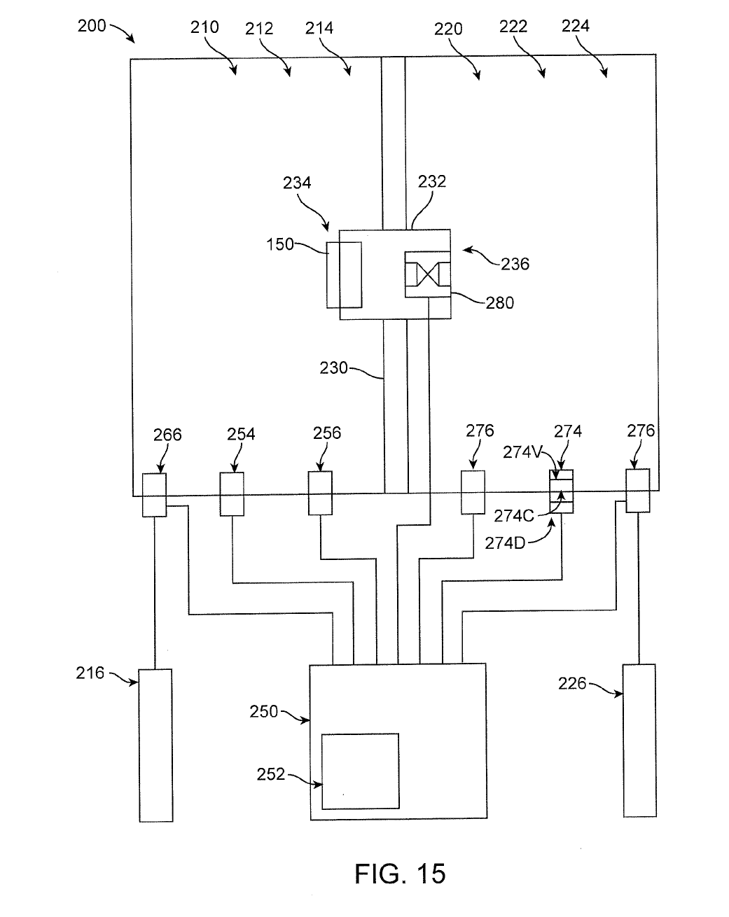

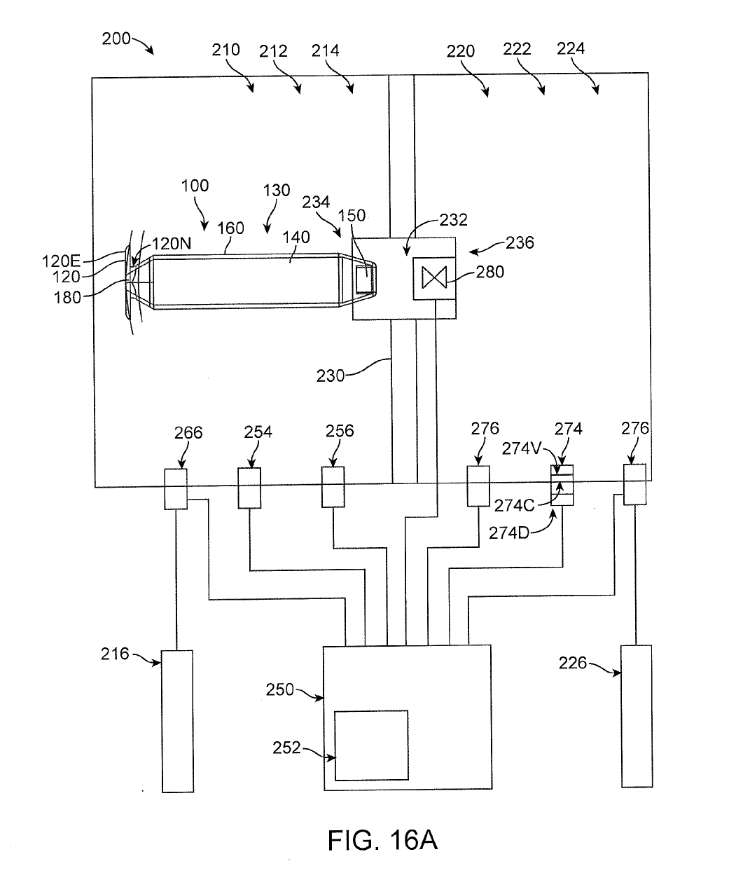

FIG. 14A shows a porous frit structure composed of sintered metal powder, in accordance with an implementation;

FIG. 14B shows a porous frit structure having sintered metal fibers, in accordance with an implementation;

FIG. 14C shows an SEM micrograph of porous structure 150 comprising sintered Titanium (Ti). The micrograph measured the portion of the structure that faces the chamber of the device 100 or faces away from the device toward the eye. The porous structure 150 comprising sintered Ti had measured a nitrogen gas flow rate of about 42 SCCM with a substantially constant pressure drop across porous structure 150. The measured rate of diffusion with Lucentis™ through similar porous Ti structures having similar gas flow was substantially greater than the estimated rate of diffusion based on the gas flow rate. The porous structure comprising sintered Ti comprised a plurality of granule sizes. Similar micrographs were obtained for similar sintered Ti porous frit structures. These data suggest that particle size and distribution can affect gas flow rates.

FIG. 15 shows an apparatus 200 to determine a release rate of a therapeutic agent through a porous structure based at least in part on diffusion. The diffusion measured may comprise one or more of diffusion of a low molecular weight ion, a low molecular weight molecule, diffusion of an incompressible fluid such as a liquid, or diffusion of a compressible fluid such as a gas. Diffusion of one or more of many gases can be measured such as hydrogen, helium, oxygen, nitrogen, or gases such as combinations of elements for example air, carbon dioxide. As the release of therapeutic agent comprises diffusion of the therapeutic agent through the porous structure, measurement of fluid diffusion through the porous structure and resistance of the porous structure to diffusion can provide very useful information to determine the release rate index of the porous frit structure. For example, a gas such as helium or water vapor can be used to measure the diffusive resistance. The diffusion of a fluid such as a gas can be driven by a concentration gradient rather than a pressure gradient, for example. This diffusional resistance measurement data may have a substantially higher correlation with RRI among a variety of porous structure materials.

The diffusion data can be combined with flow data. For example, for a given manufacturing process of a known material, known particles size and repeatable sintering process, flow among samples can be measured compared and combined with diffusion data of similar samples so as to determine the resistance to diffusion of the porous structure 100 such as the release rate index.

Measurement of the diffusional resistance of a small species in a liquid can also be used to identify porous structures with the desired properties. For example, diffusion of hydrogen ions can be much more rapid than protein diffusion. Apparatus 200 can be configured such that hydrogen ions may be generated on one side of the porous structure and the appearance of hydrogen ions can be measured with a pH probe on the other side. The rate of appearance of hydrogen ions and pH can be related to the diffusional resistance of the porous structure. Other small molecules, such as a dye, can be used to rapidly characterize the diffusional resistance of the porous structure, for example.

Test apparatus 200 comprises a first container, for example a first chamber 210 and a second container, for example a second chamber 220. Chamber 210 has a first fluid, for example first gas 212 having a first pressure 214. Second chamber 220 has a second fluid, for example a second gas 222 having a second pressure 224. A barrier 230 separates the first chamber from the second chamber. Barrier 230 has a channel 232 extending through the barrier so as to couple the first chamber and the second chamber. Channel 232 extends to a first opening 234 into the first chamber 210 and a second opening 236 extending into the second chamber 220. The opening 234 can be sized to receive the porous structure 150, and can be sized to receive at least a portion of the therapeutic device 100 such that the porous structure 150 can be tested within the therapeutic device 100.

Test apparatus 200 comprises circuitry such as a processor 250 having a computer readable memory 252 for storing instructions of a computer program so as to control testing and determine the diffusional resistance of the porous structure 150, and may have instructions to determine convective flow of a gas through the porous structure. Alternatively or in combination, the circuitry may comprise logic circuitry such as programmable array logic (hereinafter “PAL”) having instructions embodied thereon to control the testing and determine the resistance to flow, and many other steps as described herein similar to processor 250 having the computer readable memory.

Processor 250 can be coupled to at least one valve and at least one sensor to control testing of porous structure 150. A valve 280 can be located along channel 232 and coupled to processor 250 so as to open and close channel 232 in response to commands from processor 250. A flow controller valve 266 is coupled to processor 250 and gas supply 216, for example a helium supply, so as to control pressure of the gas in chamber 210 and inject the gas from supply 216. A sensor 254 is coupled to processor 250 to measure an amount of gas from supply 216 in chamber 210. A release valve 256, for example a vent, is coupled to processor 250 so as to release gas from chamber 210.

Processor 250 can be coupled to components coupled to chamber 220. A flow controller valve 276 is coupled to processor 250 and gas supply 226, for example a nitrogen supply or an air supply, so as to control pressure of the gas in chamber 220 and inject the gas from supply 226. A sensor 274 is coupled to processor 250 to measure an amount of gas from supply 226 to chamber 220. A release valve 276, for example a vent, is coupled to processor 250 so as to release gas from chamber 220.

The flow controller valve 266 and the flow controller valve 276 can compensate for pumping of sample into the detector to maintain the pressure 214 substantially similar to pressure 224. Placement of a diaphragm in the barrier 230 or a tube with a column of non-volatile liquid between chamber 210 and chamber 220 may also maintain pressure 214 substantially similar to pressure 224. The test apparatus may be temperature controlled to improve repeatability and accuracy of the results or to alter the kinetics of the gas test with temperature by changing the temperature so as to affect the gas diffusion and corresponding measured gas diffusion coefficients. Alternatively or in combination, the temperature may be monitored and used to correct the results based on the measured temperature and the temperature dependence of the diffusion coefficient.

At least one of the detectors may comprise a detector responsive to a first gas and substantially non-responsive to a second gas such as a helium detector, for example. The detector responsive to the first gas {can be comprise a }first signal in response to the first gas and such that the signal is not changed substantially by the second gas. Helium is inert and can be used for non-destructive and sensitive testing of the porous structure. The detector may comprise one or more components of commercially available helium detectors suitable for incorporation in accordance with embodiments as described herein, and may be based on mass spectrometry or other technologies such as a selective ion pump detector. (For example see www.mksinst.com and varianinc.com on the Word Wide Web).

The detector may comprise a known commerically available helium mass spectrometer leak detector modified in accordance with the embodiments as described herein. The helium detector may comprise a vacuum system to maintain adequately low operating pressure in the spectrometer tube. Exemplary maximum test port pressures for conventional detectors are on the order of 1-10 Torr. Some systems can be optimized for use at higher pressures (for example, see “Introduction to Helium Mass Spectrometer Leak Detection” on the Varian website) or can be used at atmospheric pressure (e.g., sniffer mode). Tests of gas diffusion through porous materials as described herein may be performed at pressures higher than the maximum test port pressure of at least some commercially available detectors. Helium concentrations can be measured from samples with higher pressure by use of throttling valves and other techniques known in the art. The most efficient test may utilize pressures that match the allowable pressures for the detector. A person of ordinary skill in the art can determine suitable pressures of the chambers to measure diffusion through the porous structures based on the teachings described herein.

The processor 250 may comprise instructions to measure diffusive flux with pressure 214 substantially similar, for example substantially equal to pressure 224, such that convective flow across porous structure 150 is substantially inhibited. With helium on one side of the frit and an equal pressure (atmospheric or less) of low signal gas on the other, for example nitrogen. The helium can be measured on the helium side or the low signal side, for example, and the release of helium measured. A decay test can be performed, for example by measuring an amount of helium at a time following the initial configuration of helium on one side and the low signal gas on the other side.

Detectors based on mass spectrometry can be designed so as to isolate the ions of the specified tracer gas such that transmission of other gases to the collector can be substantially inhibited. Hence, other gases can only provide a signal if they contain trace amounts of the tracer gas. Helium can be used as a tracer gas because the concentration in the atmosphere is low, only 5 parts per million. Other high purity gases with low amounts of helium can be used as the second gas so as to have an inhibited signal at the detector. For example, high purity nitrogen with no substantial amounts of helium can be used as the second gas. Alternatively or in combination, air can be used as the second gas due to the low amounts of helium in air.

A person of ordinary skill in the art can conduct experiments based on the teachings described herein so as to determine the correspondence between diffusion and release rate of therapeutic index, for example RRI.

Examples of additional flow test that can be performed with apparatus 200 or combined with measurements of apparatus 200 include:
Capillary Flow Porometer
Fuel Cell Porometer
Advanced Capillary Flow Porometer
Capillary Condensation Flow Porometer
Automated Filter Cartridge Tester
Multipoint Simultaneous Pore Structure Analyzer
Liquid-Liquid Porometer
Cartridge Bubble Point Tester
Simple Porometer
Cake Forming Porometer
In-Plane Porometer
Microflow Porometer
Clamp-On Porometer
QC Porometer
Compression Porometer
Cyclic Compression Porometer
Complete Filter Cartridge Analyzer
Integrity Analyzer
Bubble Point Analyzer
Filtration Media Analyzer
Custom Porometers

Examples of apparatus suitable for combination with apparatus 200 are commercially available from Porous Materials, Inc. (available on the world wide web at pmiapp.com and micromeritics.com)

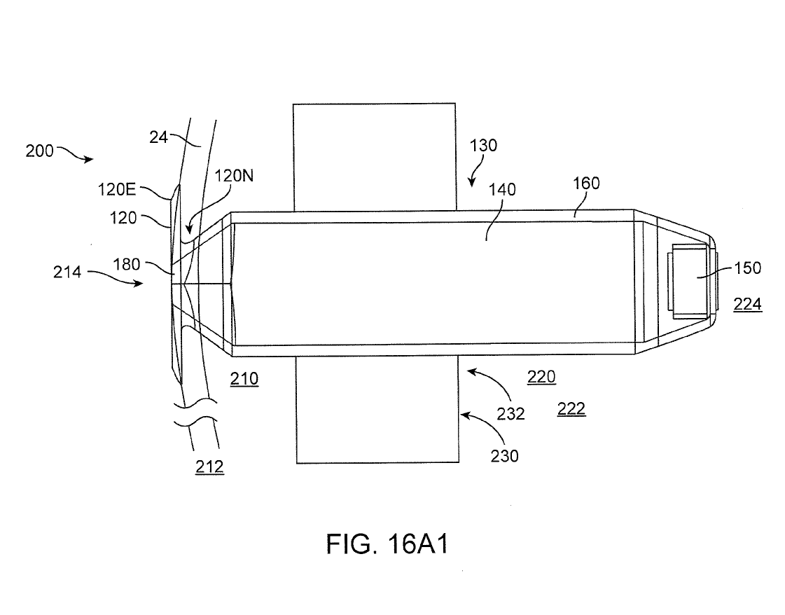

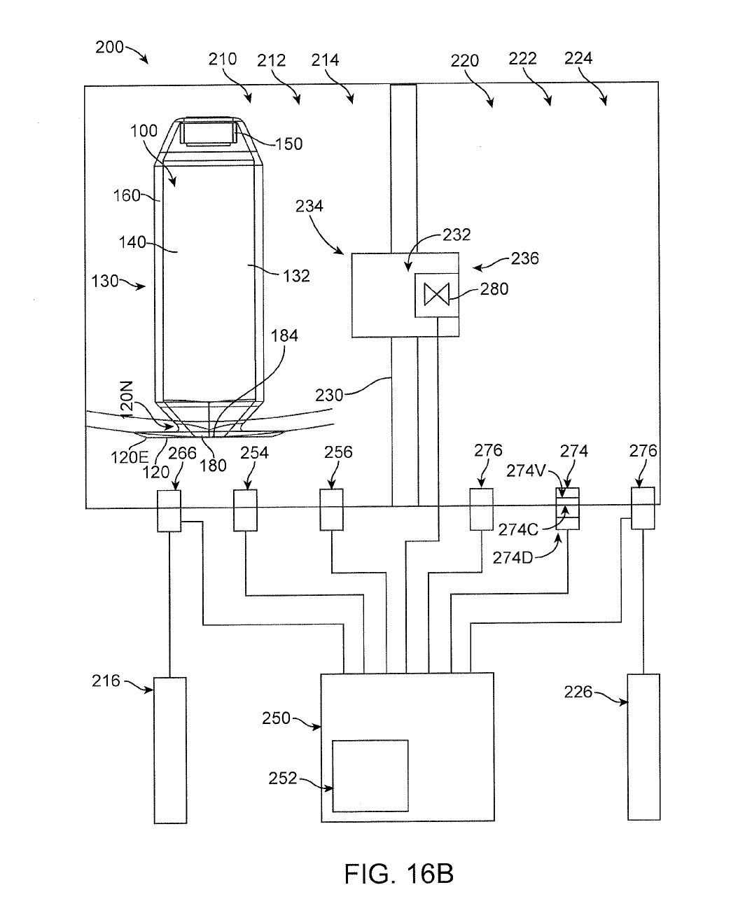

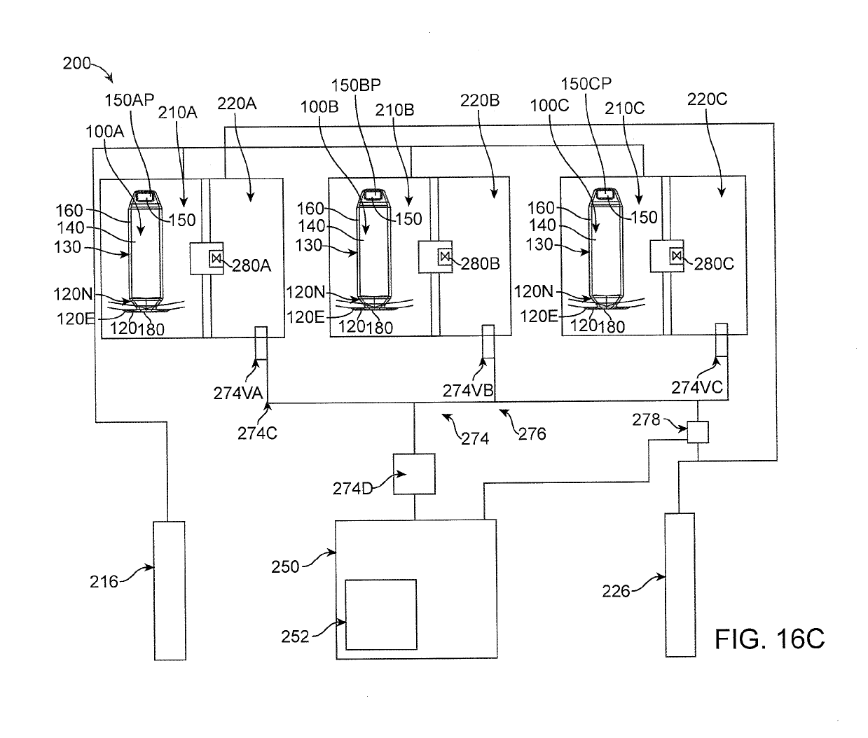

FIG. 16A shows test apparatus 200 configured to measure diffusion of a fluid through a porous structure such as porous structure 150. For example, diffusion of a gas through a porous structure can be measured in which the porous structure is coupled to a housing of the therapeutic device when the housing is mounted in the test apparatus. The test apparatus 200 can be sized and configured to test the porous structure when therapeutic device 100 is at least partially assembled, for example when porous structure 150 is mounted to a housing of the porous structure. The mount may be designed of a thickness, such as one or more mm, and a low gas permeability material, such as neoprene or nitrile rubber, so as to minimize background signal due to penetration of the first gas or the second gas. The mount may also comprise a shape so as to fit the porous structure or housing of the device so as to seal the porous structure when placed in the mount.

The chamber of therapeutic device 100 can be filled with a test gas, for example helium, and release of helium to chamber 220 can be measured. For example, therapeutic device 100 can be substantially assembled including port 180 without the penetrable barrier and chamber 210 filled with helium to fill the chamber of the therapeutic device when valve 280 is closed. The chamber 220 may comprise a second gas, and valve 280 opened to couple the first chamber to the second chamber through channel 232 with porous structure 150 extending substantially across opening 234. Alternatively or in combination, decreased concentration of helium in chamber 210 can be measured when diffusion of gas from chamber 220 into chamber 210 decreases the concentration of the gas in chamber 210. For example, nitrogen from chamber 220 can diffuse into chamber 210 and decreased amounts of helium in chamber 210 can be measured, and the rate of decrease can be related to the resistance of porous structure 150 to flow. The resistance to diffusion can be correlated with the release rate index.

FIG. 16A1 shows the housing of therapeutic device 100 extending substantially into opening 234 so as to measure the therapeutic device with porous structure 150 located within channel 232. Opening 234 can be sized to receive the housing of therapeutic device 100.

FIG. 16B shows the assembled therapeutic device 100 placed in the first container, for example first chamber 210. The assembled therapeutic device 100 may comprise the porous structure 150 on a first end and the penetrable barrier 184 disposed on the second end, such that the diffusive resistance of the assembled device can be measured with the porous structure 150 and penetrable barrier 184 placed on the device 100 so as to define the volume of the reservoir chamber. The test apparatus 200 can comprise chamber 210 sized to receive the assembled therapeutic device 100, such that the assembled therapeutic device 100 can be placed in chamber 210. The therapeutic device may comprise the penetrable barrier 184, for example a septum, located on a first end and the porous structure 150 located on a second end. Initially, chamber 210 and 220 can be evacuated by vacuum. Chamber 210 can be filled with helium for a period of time so as to pressurize chamber 210 with helium and provide helium of an intended pressure to the chamber of therapeutic device 100 through the porous structure. After an amount of time valve 266 to the supply 216 of helium is shut. The pressure of chamber 210 can be monitored until the pressure approaches a substantially constant value, indicating helium has equilibrated inside and outside of the drug delivery device; i.e., on both sides of the porous structure within first chamber 210. A gas other than helium, for example air or nitrogen, can be fed into chamber 220 until the pressure 224 of the second chamber is substantially similar to pressure 214. A diaphragm or liquid filled column can couple the first chamber 210 to the second chamber 220 so as to provide pressure equalization. {At time an initial time}, for example time zero, the valve 280 can be opened so as to allow helium to diffuse from chamber 210 to chamber 220. The chamber 210 can be shape such that the volume of the chamber of the therapeutic device 100 comprises a majority of the gas volume of chamber 210 when device 100 is placed in chamber 210. The larger fractional of volume of the chamber 210 that is occupied by the device 100, the more the diffusional resistance of porous structure will contribute to the rate of helium diffusing into chamber 220. Helium can be allowed to accumulate in chamber 220 for an intended amount of time, after which the valve 280 is closed. The amount of helium in chamber 220 can be measured when valve 280 is closed after the intended amount of time.

The sensor 274 may comprise a valve 274V and a detector 274D, each coupled to processor 250. A channel 274C can extend between valve 274V and detector 274D. Valve 274V can be opened so as to couple the detector 274D to the chamber 220 to determine the amount of helium in chamber 220. The valve 274V can be opened so as to connect chamber 220 to the detector 274D comprising the helium detector, such that helium can be drawn into the detector for quantization. The amount of helium transferred into chamber 220 is related to the diffusional resistance of the porous structure 150 of the therapeutic device, for example the RRI. The amount of helium can also be related to the volume of the chamber of the therapeutic device 100 such that the tuning of the porous structure 150 and the volume of the therapeutic device to an intended volume of a formulation of therapeutic agent can be measured.

It is contemplated that the test apparatus can be built with multiple chambers so as to increase throughput. The apparatus 200 may comprise a plurality of first and second chambers, such that the gas sources and the detector can cycle among the plurality of first and second chambers. An advantage of this test scheme is that many final devices can be tested without puncturing the penetrable barrier 184 comprising the septum.

The one or more of the gas diffusion or gas flow can be measured in many ways based on the teachings as described herein. For example, the needle 189 as described herein can be used to inject a gas into the assembled device 100, as shown in FIG. 7 to FIG. 7B-61, for example. The gas injected into device 100 can be used to measure the flow of the gas based on pressure of the gas injected into the device chamber, and the diffusion of the gas from the device 100 through the porous structure can be measured to determine the release rate index for drug release, for example. The measured diffusion of the porous structure 150 can be a measured diffusion of gas into the chamber of device 100, or the measured diffusion may comprise diffusion of the injected gas out of the chamber through the porous structure 150, for example.

The data for the amounts of gas of the first chamber, for example helium, can be related to diffusion properties of the porous structure 150 that are similar to the diffusion of the therapeutic agent. The above equation for release of therapeutic agent is expressed as:

c_{R}=c_{R0} exp((−D PA/FL V_{R})t)
and can be modified so as to correspond with the gas where
c_{R} is the concentration of gas
c_{R0} is the initial concentration
D is the diffusion constant/coefficient for the gas
P is the porosity
A is the area
F is a channel fit parameter that may correspond to the tortuosity of the porous frit structure
L is the thickness
V_{R} is the volume of the first chamber, for example the reservoir and
t is the time.

The cumulative Release=1−c_{R}/c_{R0}

The half-life of the gas corresponds to the time for the concentration to decrease to one-half of an initial value. The ratio of the diffusion coefficients can be used to determine the half-life of the therapeutic agent based on the measured half-life of the gas diffused from the therapeutic device.

(Half-life with Agent 110)=(Measured half-life with helium)*(Dgas)/(Dta)

Where Dgas is the diffusion coefficient of the measured gas and Dta is the diffusion coefficient of the therapeutic agent.

The diffusion coefficient of gas at 1 atm and room temperature (about 290K) can be within a range from about 0.1 to 1 cm²/s, and can depend on the idenity of the gases when the gas comprises a mixture. For a binary mixture, the diffusion coefficients of each gas can be substantially equal. For example, the diffusion coefficient for both helium and nitrogen in a helium nitrogen mixture can be about 0.69 cm2/s, and the diffusion coefficient can be about 0.61 cm2/s for helium and carbon dioxide in a helium carbon dioxide mixture. For the therapeutic agent 110 in a liquid, the diffusion coefficient can be about 1×10⁻⁶ cm²/s for proteins such as Lucentis™ (ranibizumab) at about 37 C. As the half-life is inversely proportional to the diffusion coefficient, a device with an effective half-life of protein of about 100 days (8.6×10⁶ s) corresponds to a half-life of about 10 seconds for helium gas such that gas diffusion can provide rapid determination of diffusion data through porous structure 150.

As an example in accordance with embodiments, the half-life of helium gas in the device 100 can be measured and determined to be about 10 s. Based on the above equation,

Half-life of ranibizumab=(10)*(1)/(1×10⁻⁶)=10⁷ s=115.7 days.

Additional gases such as CO2 and others having known diffusion coefficients can be used, and at least some gasses may comprise a diffusion coefficient that is about one tenth the diffusion coefficient of helium. For example, at 1 atm and room temperature, the diffusion constant of CO2 is about 0.61 in a mixture of carbon dioxide and helium. The diffusion constant of CO2 is about 0.13 in a mixture of carbon dioxide and argon. The timing of the measurements and delays as described herein can be adjusted based on one or more of the gasses used, the ratio of gases of a mixture, the diffusion coefficient, the temperature, or the pressure. Many gases as described herein can be used to determine the release of the therapeutic agent from the porous structure of the device 100 based on gas diffusion.

FIG. 16C shows a plurality of assembled therapeutic devices placed in a plurality of containers, for example a plurality of chambers. The plurality of therapeutic devices comprises a first therapeutic device 100A, a second therapeutic device 100B, and a third therapeutic device 100C, similar to therapeutic device 100. Each therapeutic device comprises a porous structure 150 corresponding to a plurality of porous structures 150AP, 150BP and 150CP. Each therapeutic device may comprise a penetrable barrier 184 and a container that defines a chamber as described herein. One or more of the pressure or fluid concentration gradient can be controlled so as to determine the tuned response of the chamber and porous structure.

The plurality of chambers comprises chamber 210A, chamber 210B and chamber 210C similar to chamber 210. The first plurality of chambers can be coupled to a second plurality of chambers. The second plurality of chambers comprises a chamber 220A, chamber 220B and chamber 220C similar to chamber 220. A plurality of valves is coupled between the plurality of chambers to couple the first plurality of chambers to the second plurality of chambers when opened and isolate the first plurality of chambers from the second plurality of chambers when closed. The plurality of valves comprises valve 280A, valve 280B and valve 280C similar to valve 280.

The first plurality of chambers can be connected to a first supply of a first fluid with valves coupled to the processor 250, and the second plurality of chambers can be connected to the second supply of the first fluid with valves coupled to the processor 250 as described herein.

A fluid sensor 274 may comprise a second plurality of valves 274VA, 274VB and 274VC. The second plurality of valves 274VA, 274VB and 274VC are coupled to the detector 274D with a channel 274C extending between the plurality of valves and the detector. Each of the second plurality of valves 274VA, 274VB and 274VC, is coupled to one of the second chambers. Each valve can be opened and closed independently under control of processor 250 so as to open and close the valves selectively, for example so as to sequentially couple one of the second chambers to the detector 274D for measurement of the fluid accumulated in the second chamber similar to chamber 220. The detector 274D is coupled to processor 250 so as to measure the amount of gas in each of the second chambers.

The channel 274C can be cleared with a purge valve 278 to prepare the channel 274C to receive the fluid from each of the second chambers. Alternatively or in combination, a vacuum pump coupled to one or more valves can be connected to one or more of the chambers or channels so as to purge the one or chambers or channels of gas, for example so as to prepare the channel 274C to receive the fluid from each of the second chambers. A vacuum pump and valve can also be coupled to each of the first chamber and the second chamber so as to purge gas from the chamber prior to providing gas. For example, the first chamber may be purged of gas then filled with helium.

The processor 250 can be configured in many ways to measure the chamber and porous structure of each therapeutic device. For example, the processor can be configured to measure diffusion of the fluid from each of the plurality of therapeutic devices when placed in the first plurality of chambers. For example, the first chamber and the device chamber may comprise a first gas and the second chamber may comprise a second gas, and the diffusion of the gas from the device chamber to the second chamber measured with opening of valve 280. Alternatively or in combination, the therapeutic device chamber and the first chamber may comprise a first and the second chamber 220 may comprise a second pressure different from the first pressure when valve 280 is closed, and processor 250 can be configured to measure changes in pressure when the valve 280 is opened.

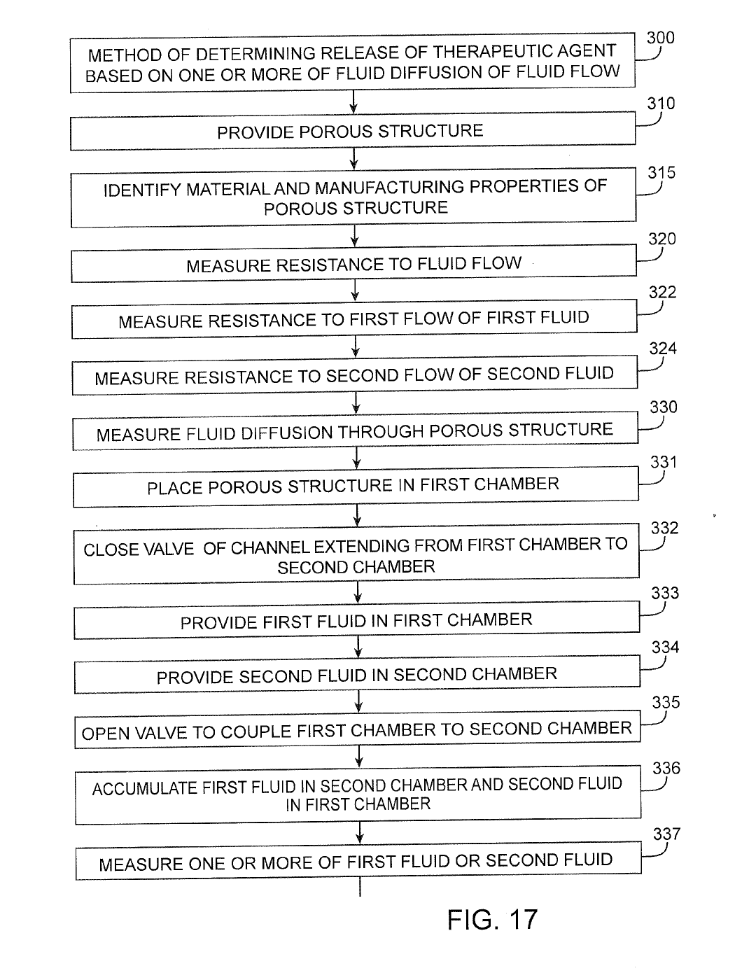

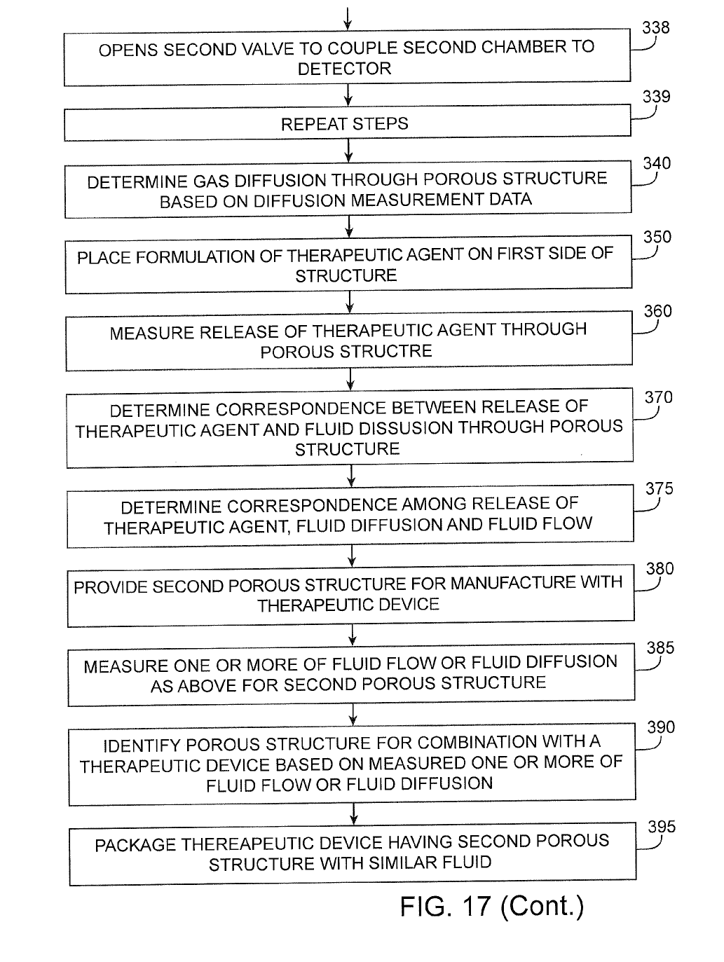

FIG. 17 shows a method 300 of identifying a porous structure of a therapeutic device in accordance with embodiments. The method 300 may comprise a method of determining release of therapeutic agent based on one or more of fluid diffusion or fluid flow.

A step 310 provides a porous structure, for example porous structure 150 as described herein.

A step 315 identifies material and manufacturing properties of the porous structure.

Ti may show about 1.5× increase in RRI as compared to SS for comparable flow rates and an adjustment to RRI can be made based on flow rate and material, in accordance with embodiments as described herein, for example.

A step 320 measures resistance to fluid flow.

A step 322 measures resistance to first flow of a first fluid. The first fluid can be liquid or a gas having a first viscosity.

A step 324 measures a second resistance to flow of a second fluid. The second fluid can be a liquid or a gas having a second viscosity, for example.

A step 330 measures fluid diffusion through the porous structure, for example gas diffusion.

A step 331 places the porous structure in a first container, for example a first chamber.

A step 332 closes a valve of a channel extending from a first container to a second container, for example from a first chamber to a second chamber.

A step 333 provides a first fluid on a first side of the porous structure, for example a first gas on the first side of the porous structure.

A step 334 provides a second fluid on a second side of a porous structure, for example a second gas.

A step 335 opens a valve to couple the first container to the second container, for example to couple a first chamber to a second chamber.

A step 336 accumulates the first fluid in the second container and the second fluid in the first container, for example the first gas in the second chamber and the second gas in the first chamber.

A step 337 measures one or more of the first fluid or the second fluid, for example measures one or more of a first gas or a second gas.

A step 338 opens a second valve to copule the second chamber to a detector, for example opens the second valve to measure an amount of first gas accumulated in the second chamber.

A step 339 repeats one or more of the above steps.

A step 340 determines diffusion through the porous structure based on diffusion measurement data, for example gas diffusion through the porous structure based on diffusion measurement data.

A step 350 places a formulation of therapeutic agent on the first side of the porous structure.

A step 360 measures release of therapeutic agent through the porous structure.

A step 370 determines correspondence between release of the therapeutic agent and fluid diffusion through the porous structure.

A step 380 provides a plurality of porous structures for manufacture with the therapeutic device.

A step 385 measures one or more of fluid flow or fluid diffusion of the plurality of porous structures, for example one or more of gas flow or gas diffusion as described herein.

A step 390 identifies one or more of the porous structures of the plurality as suitable for combination with a reservoir component of a therapeutic device based on one or more of fluid flow or fluid diffusion. For example, the identified porous structure can be combined with a component of a therapeutic device to provide a therapeutic device having a known chamber volume.

A step 395 packages the therapeutic device having the identified porous structure with a similar fluid. For example, when diffusion is measured with a gas such as helium, the therapeutic device 100 can be packaged with a gas such as nitrogen. When diffusion is measured with a substantially incompressible fluid such as a liquid, the therapeutic device can be packaged with a liquid.

The apparatus 200 and method 300 can measure diffusion in many ways. For example, the first fluid may comprise a substantially incompressible fluid such as a first liquid and the second fluid may comprise a substantially incompressible fluid such as a second liquid, in which the first liquid can be {miscible} with the second liquid. For example, the first liquid may comprise a first solvent and the second liquid may comprise a second solvent and the accumulation of the first solvent in the second chamber measured.

The diffusion measured with apparatus 200 and method 300 can be diffusion of a small molecule, for example a proton ion, in a liquid such as water, as the diffusion coefficient for a small low molecular weight ion in water can be substantially greater than a large molecule such as ranibizumab. For example, the first chamber can be filled with a first fluid, comprise a liquid having a first pH and the second chamber can be filled with a second solution having a second pH, and the valve 280 can be opened and the pH measured in the second chamber.

It should be appreciated that the specific steps illustrated in FIG. 17 provide a method of measuring a porous structure, according to an implementation. Other sequences of steps may also be performed according to alternative embodiments. For example, alternative implementations may perform the steps outlined above in a different order. Moreover, the individual steps illustrated in FIG. 17 may include multiple sub-steps that may be performed in various sequences as appropriate to the individual step. Furthermore, additional steps may be added or removed depending on the particular applications. One of ordinary skill in the art would recognize many variations, modifications, and alternatives.

Many of the above steps can be implemented with instructions stored with a computer readable memory of the processor of the apparatus 200. The instructions stored in the memory of the processor of apparatus 200 may comprise instructions to perform many of the steps of method 300.

The above method may comprise an algorithm to determine frit, can be based on frit characteristics, and prior measured RRI, e.g., Ti or SS frit material identified, and also based on flow tests.

The Algorithm to determine RRI based on flow can be based on one or more of the following: different material properties of Ti and SS, such as one or more of increased chemical reactions of SS, increased surface adsorption of SS, increased surface area of SS; different gas flow characteristics for similar diffusive characteristics (such that flow test can be adjusted or RRI needs to be adjusted), may have fiber structure for Ti instead of granules such that flow through Ti is impeded less than through SS, also pressure drop may increase for smaller holes with same surface area as larger holes.

Additional considerations can be that the porous Ti can have a surface sheet that may decrease flow with inhibited change in RRI based on the masking study as described herein with reference to the publication and patent previously incorporated by reference and, dead end channels of the porous structure.

The materials as described herein can be characterized so as to accommodate changes to the porous frit structure material to provide increased stability of Lucentis™ for extended times.

The following porous sintered structure parameters can be adjusted so as to provide a release rate index: porosity, dimensions including length and width, particle size and distribution of particle size, temperature and compression of particles, increase humidity or temporary addition of a gas or liquid so as to reduce interparticle interaction and increase density when particles are compacted with or without vibration, roughness of particles, channel opening size and diameters (e.g., mesh or coating on surface, or slip surface with holes decreasing area of pores on surface), different shapes of particles such as granules or fibers, preprocessing to passivated. Based on the teachings as described herein, to lower RRI decrease A, increase T, decrease porosity.

The tortuosity can be related to the diffusion and convective flow data.

Titanium and many materials may be made from rod or fiber-like structures, and convective streamlines may be insensitive to some of the gaps between the fibers; i.e., not much air may flow in the gaps behind where the convective flow is impinging on the fibers. However, diffusion can be able to take advantage of these extra connections. The porous structure with rods may have a smaller diffusive tortuosity than its effective convective tortuosity. The porous structure with rods may have less diffusive resistance than convective resistance, which can be related to the shift between RRI and gas flow.

Sintered fibers may comprise negative of sintered spheres. The fibers can be interconnected and surrounded by a continuum of empty space vs. pores of empty space interconnected and surround by a continuum of metal. The tortuosity from these two cases can be different.

To efficiently achieve slow release a high tortuosity can be helpful. This can be achieved by interconnected, tortuous air pores surrounded by a continuum of metal. If the porous titanium structure is made from rods, for example, one can adjust the RRI based on flow that corresponds to sintered fiber to tortuous air pores by changing the particle shape from rods to something more spherical. Or add particles of smaller size, preferably spherical, to fill in the gaps between the fibers.

The alternative may also be used in accordance with embodiments described herein. For high drug release rates from a drug suspension, a fiber structure may be used. The gaps between the fibers can be chosen small enough so as to maintain the particles of the suspension, for example crystals, in the therapeutic device reservoir chamber without flushing out of the device when the reservoir chamber is refilled. The continuum of empty space around the fibers can enable high diffusive fluxes.

A two layer structure may be advantageous for slow release of protein. A first, sintered fiber layer can trap particulates with less clogging and less impact on RRI because of the continuum of empty space. Then a second layer that has tortuous air pores can efficiently produce a reduced diffusive flux.

Although the gas flow model may not exactly correlate with diffusion through frit structures, the gas flow model can be used in accordance with embodiments as described herein. Model development may include pores size for gas flow that may not be important for diffusion, for example due to increase frictional drag of increased surface area of decreased channel sizes, for example when porosity remains substantially constant, and could also have increased frictional drag due to increased roughness of surface area that can decrease convective flow more than diffusion.

Work in relation to embodiments indicates that diffusion testing as described herein can be used to measure diffusion of a tuned therapeutic device 100. The tuned device may comprise the chamber and porous structure, and the tuned diffusion. For the tuned release of ranibizumab having a device half-life of at least about thirty days, the tuned diffusion of a gas may comprise a half-life of no more than about 60 seconds when measured with diffusion, for example.

For example, the diffusion coefficient of gas at 1 atm and room temperature is about 1 cm²/s, whereas the diffusion coefficient can be about 1×10⁻⁶ cm²/s for proteins such as Lucentis at about 37 C. Devices with effective half-life of protein of about 30 and 100 days correspond to half-life of about 3 and 9 seconds for helium gas at room temperature. Since diffusion coefficients are roughly inversely proportional to pressure, for a device with protein half-life of 100 days would have a gas half-life of 4 seconds at 380 Torr and 0.1 seconds at 10 Torr. The diffusion coefficient would also depend on temperature, changing by approximately 5-10% for a temperature change of 10° C. Variables such as pressure and temperature can be changed to vary the kinetics of the gas diffusion measurement for a given therapeutic device.

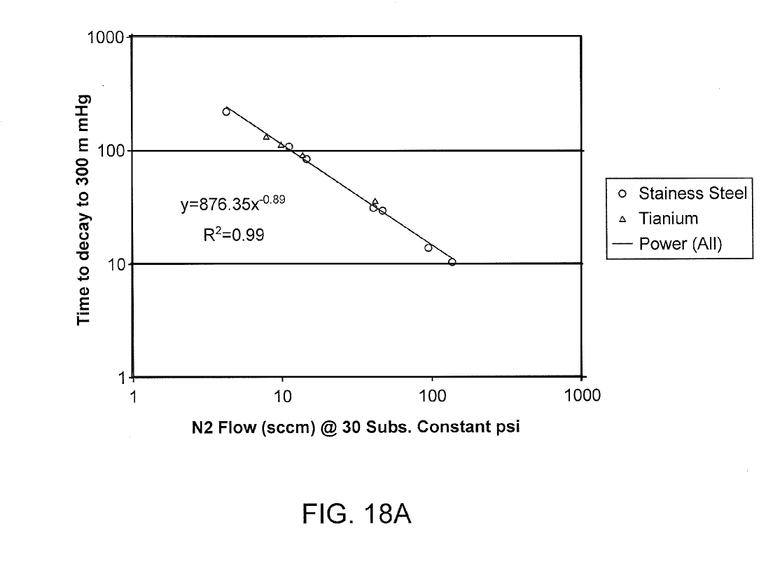

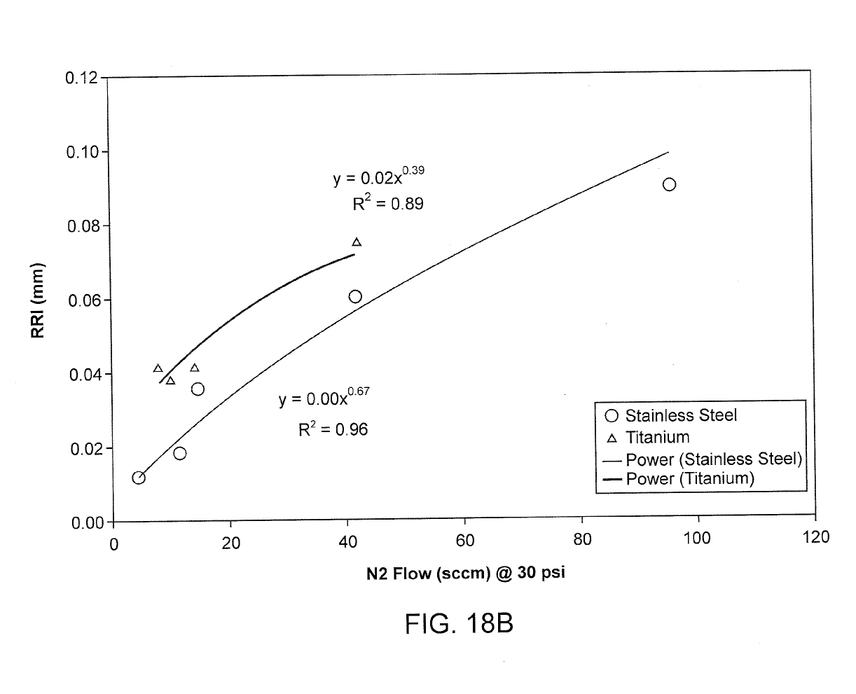

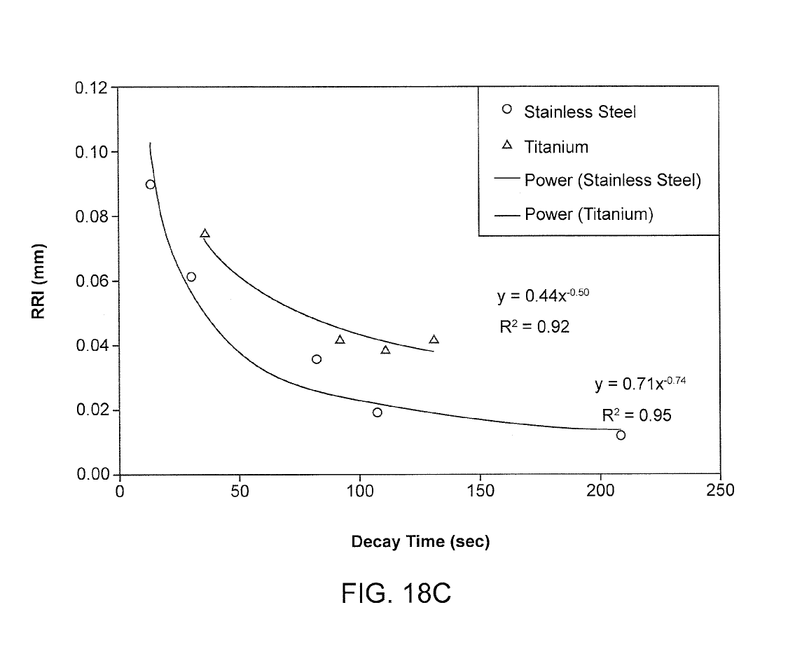

FIGS. 18A to 18C show a comparison of flow rate data and RRI's for sintered titanium and sintered stainless steel.

FIG. 18A shows a comparison of flow rate data commercially available from Mott Corporation to a decay time test to determine the gas flow through porous frit structures. These data are highly correlated and show a fit to a power curve with an R2 of 1.0.

FIG. 18B shows a comparison of flow rate data as in FIG. 39 to RRI for Ti and SS porous frit structures. These data show that Titanium is more permeable to diffusive mass flux than convective air flow as compared to SS. The increased diffusive mass flux can correspond to an increased release rate index for the Ti porous structures as compared to SS porous structures having comparable N2 flow at a substantially constant pressure within a range from about 10 to about 50 PSI.

FIG. 18C shows a comparison of decay time data as in FIG. 39 to RRI for Ti and SS porous frit structures. These data show that Titanium is more permeable to diffusive mass flux than convective air flow as compared to SS. The increased diffusive mass flux can correspond to an increased release rate index for the Ti porous structures as compared to SS porous structures having comparable N2 decay time.

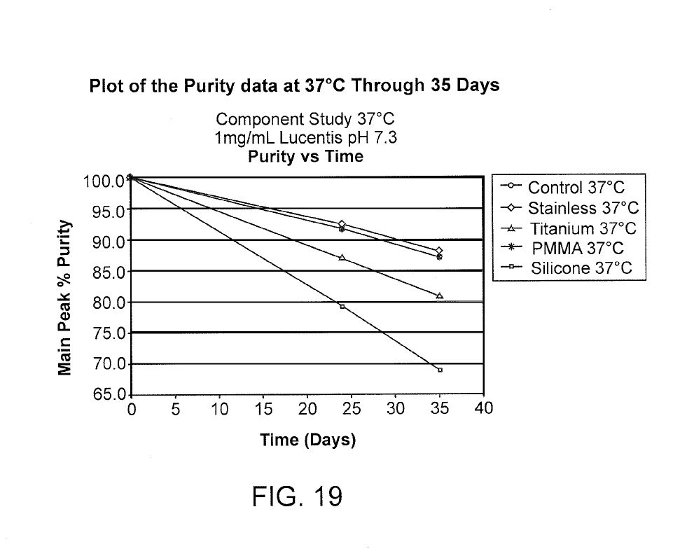

FIG. 19 shows stability data for a formulation of Lucentis™ that can be used to identify materials for porous frit structures. These data show the stability of Lucentis™ over time for containers having materials such as stainless steel, Ti, PMMA and silicone. These data were measured with ion exchange chromatography, and can be measured in accordance with published references describing Mab patterns on SCX-10 column.

The data below was generated with the following method:

Waters HPLC system.

1-2 mg/mL protein concentration.

Injection Volume 10-50 uL

Dionex SCX-10 Strong Cation Exchange Column

20 mM Phosphate Buffer System pH 3.6

1 M NaCl Gradient from 1-99% in 30 minutes. Flow Rate: 1 mL/min

UV Absorbance @ 214 nm. Column Temperature: 45° C.
The method is in accordance with references on the Dionex website, such as:
Title: MAbPac SCX-10 Column for Monoclonal Antibody Variant Analysis
(available on the world wide web at dionex.com/en-us/webdocs/87008-DS-MAbPac-SCX-10-Column-20Aug2010-LPN2567-03.pdf)
Title: Monitoring Monoclonal Antibody Heterogeneity by Cation Exchange Chromatography.
(available on the world wide web at dionex.com/en-us/webdocs/4470-AN127-Cation-Exchange-Chromatography-02Feb09-LPN1047-01.pdf)

Table 3 shows recovery and stability of Lucentis with materials that can be used for porous structure 150 as described herein. Additional testing of additional materials can be performed, for example with one or more ceramic materials. Table 3 shows Ion Exchange Chromatography of Lucentis aged at 37° C. in contact with device components for 35 days. Lucentis was diluted to a concentration of 1 mg/mL ranibizumab in PBS, with final pH of 7.3. Recovery was corrected for evaporative water loss during the 35 day study (8.0%).

TABLE 3
RECOVERY AND STABILITY OF LUCENTIS WITH
MATERIALS FOR POROUS STRUCTURES
Component Study 37° C. - 35 Days

| Sample | % Recovery | Average % Purity |
|---|---|---|
| Control 37° C. | 98.1 | 87.3 |
| Stainless 37° C. | 89.5 | 68.8 |
| Titanium 37° C. | 96.2 | 80.8 |
| PMMA 37° C. | 97.8 | 88.2 |
| Silicone 37° C. | 98.0 | 87.3 |

The above data indicate that Titanium (Ti), acrylate polymer such as PMMA, or siloxane such as silicone may provide increased stability as compared to stainless steel in at least some instances. Similar testing can be performed on additional materials as described herein, for example with one or more ceramic materials.

Many ceramic materials are available, and the porous structure 150 may comprise one or more materials. The ceramic material may comprise a range of compositions, such as a porous ceramic commercially available from HP Technical Ceramics, Sheffield, UK (available on the world wide web at tech-ceramics.co.uk/mi.htm). The ceramic may comprise fused silica or borosilicate glass, for example. The ceramic may comprise a known glass or fused silica, and may comprise a highly resistant, borosilicate glass with comprising silica and boron oxide, such as USP Type I glass, for example. This ceramic material comprising silica and boron oxide can substanially decrease reactivity of the porous structure and may also have low protein adsorption. Sintered materials with smooth surfaces may also have less protein adsorption and less chemical instability mediated by the adsorption process.

Many structures or combinations of structures or method steps or components or combinations thereof as described herein can be combined in accordance with embodiments as described herein, based on the knowledge of one of ordinary skill in the art and teachings described herein. In addition, any structure or combination of structures or method steps or components or combinations thereof as described herein may be specifically excluded from any embodiments, based on the knowledge of one of ordinary skill in the art and the teachings described herein.

TABLE 1A
Therapeutic Agent List

| | Brands | | | Molecular |
|---|---|---|---|---|
| Generic Name | (Companies) | Category | Indication | Weight |
| 2-Methoxyestradiol | (Paloma | Angiogenesis | AMD | |
| analogs | Pharmaceuticals) | inhibitors | | |
| 3-aminothalidomide | | | | |
| 13-cis retinoic acid | Accutane TM | | | |
| | (Roche | | | |
| | Pharmaceuticals) | | | |
| A0003 | (Aqumen | A0003 | AMD | |
| | BioPharmaceuticals) | | | |
| A5b1 integrin | (Jerini Ophthalmic); | Inhibitors of a5b1 | AMD | |
| inhibitor | (Ophthotech) | integrin | | |
| Abarelix | Plenaxis ™ (Praecis | Anti-Testosterone | For palliative treatment | 37731 |
| | Pharmaceuticals) | Agents; | of advanced prostate | |
| | | Antineoplastic | cancer. | |
| | | Agents | | |
| Abatacept | Orencia ™ (Bristol- | Antirheumatic | For the second line | 37697 |
| | Myers Squibb) | Agents | reduction of the signs | |
| | | | and symptoms of | |
| | | | moderate-to-severe | |
| | | | active rheumatoid | |
| | | | arthritis, inducing | |
| | | | inducing major clinical | |
| | | | response, slowing the | |
| | | | progression of | |
| | | | structural damage, and | |
| | | | improving physical | |
| | | | function in adult | |
| | | | patients who have | |
| Abciximab | ReoPro ™; | Anticoagulants; | For treatment of | 42632 |
| | ReoPro ™ | Antiplatelet Agents | myocardial infarction, | |
| | (Centocor) | | adjunct to | |
| | | | percutaneous | |
| | | | 82oronary intervention, | |
| | | | unstable angina | |
| ABT-578 | (Abbott | Limus Immunophilin | | |
| | Laboratories) | Binding | | |
| | | Compounds | | |
| Acetonide | | | | |
| Adalimumab | Humira ™ (Abbott | Antirheumatic | Uveitis, AMD | 25645 |
| | Laboratories) | Agents; | | |
| | | Immunomodulatory | | |
| | | Agents | | |
| Aldesleukin | Proleukin ™; | Antineoplastic | For treatment of adults | 61118 |
| | Proleukin ™ (Chiron | Agents | with metastatic renal | |
| | Corp) | | cell carcinoma | |
| Alefacept | Amevive ™ | Immunomodulatory | For treatment of | 42632 |
| | | Agents; | moderate to severe | |
| | | Immunosuppressive | chronic plaque | |
| | | Agents | psoriasis | |
| Alemtuzumab | Campath ™; | Antineoplastic | For treatment of B-cell | 6614 |
| | Campath ™ (ILEX | Agents | chronic lymphocytic | |
| | Pharmaceuticals | | leukemia | |
| | LP); MabCampath ™ | | | |
| Alpha-1-proteinase | Aralast ™ (Baxter); | Enzyme | For treatment of | 28518 |
| inhibitor | Prolastin ™ (Talecris | Replacement | panacinar emphysema | |
| | Biotherapeutics C | Agents | | |
| | formerly Bayer) | | | |
| Alteplase | Activase ™ | Thrombolytic | For management of | 54732 |
| | (Genentech Inc) | Agents | acute myocardial | |
| | | | infarction, acute | |
| | | | ischemic strok and for | |
| | | | lysis of acute | |
| | | | pulmonary emboli | |
| AMG-1470 | | | | |
| Anakinra | Kineret ™ (Amgen | Anti-Inflammatory | For the treatment of | 65403 |
| | Inc) | Agents, Non- | adult rheumatoid | |
| | | Steroidal; | arthritis. | |
| | | Antirheumatic | | |
| | | Agents; | | |
| | | Immunomodulatory | | |
| | | Agents | | |
| Anecortave acetate | | | | |
| Angiostatin | | | | |
| Anistreplase | Eminase ™ (Wulfing | Thrombolytic | For lysis of acute | 54732 |
| | Pharma GmbH) | Agents | pulmonary emboli, | |
| | | | intracoronary emboli | |
| | | | and management of | |
| | | | myocardial infarction | |
| Anti-angiogenesis | (Eyecopharm) | Anti-angiogenesis | AMD | |
| peptides | | peptides | | |
| Anti-angiogenesis | (TRACON Pharma) | Anti-angiogenesis | AMD | |
| antibodies, | | antibodies | | |
| TRC093, TRC105 | | | | |
| Anti-angiogeric | Icon-1 ™ (Iconic | Anti-angiogeric | AMD | |
| bifunctional protein | Therapeutics) | bifunctional protein, | | |
| | | Icon-1 | | |
| Anti-endothelial | | | | |
| growth factor | | | | |
| Antihemophilic | Advate ™; | Coagulants; | For the treatment of | 70037 |
| Factor | Alphanate ™; | Thrombotic Agents | hemophilia A, von | |
| | Bioclate ™; | | Willebrand diseae and | |
| | Helixate ™; Helixate | | Factor XIII deficiency | |
| | FS ™; Hemofil M ™; | | | |
| | Humate-P ™; | | | |
| | Hyate:C ™; Koate- | | | |
| | HP ™; Kogenate ™; | | | |
| | Kogenate FS ™; | | | |
| | Monarc-M ™; | | | |
| | Monoclate-P ™; | | | |
| | ReFacto ™; | | | |
| | Xyntha ™ | | | |
| Antithymocyte | Genzyme); | Immunomodulatory | For prevention of renal | 37173 |
| globulin | Thymoglobulin ™ | Agents | transplant rejection | |
| | (SangStat Medical | | | |
| Anti-hypertensive | (MacuCLEAR) | Anti-hypertensive | AMD | |
| MC1101 | | MC1101 | | |
| Anti-platelet | | | | |
| devired growth | | | | |
| factor | | | | |
| Anti-VEGF | (Neurotech); | Anti-VEGF | AMD | |
| | Avastin ™ | | | |
| | (NeoVista) | | | |
| AP23841 | (Ariad) | Limus Immunophilin | | |
| | | Binding | | |
| | | Compounds | | |
| ARC1905 | Ophthotech | Complement | | |
| | | Cascade Inhibitor | | |
| | | (Factor C5) | | |
| Aprotinin | Trasylol ™ | Antifibrinolytic | For prophylactic use to | 90569 |
| | | Agents | reduce perioperative | |
| | | | blood loss and the | |
| | | | need for blood | |
| | | | transfusion in patients | |
| | | | undergoing | |
| | | | cardiopulmonary | |
| | | | bypass in the course of | |
| | | | coronary artery bypass | |
| | | | graft surgery who are | |
| | | | at an increased risk for | |
| | | | blood loss and blood | |
| | | | transfusio | |
| Arcitumomab | CEA-Scan ™ | Diagnostic Agents; | For imaging colorectal | 57561 |
| | | Imaging Agents | tumors | |
| Asparaginase | Elspar ™ (Merck & | Antineoplastic | For treatment of acute | 132.118 |
| | Co. Inc) | Agents | lympocytic leukemia | |
| | | | and non-Hodgkins | |
| | | | lymphoma | |
| Axitinib | | Tyrosine Kinase | | 386 |
| | | Inhibitors | | |
| Basiliximab | Simulect ™ (Novartis | Immunomodulatory | For prophylactic | 61118 |
| | Pharmaceuticals) | Agents; | treatment of kidney | |
| | | Immunosuppressive | transplant rejection | |
| | | Agents | | |
| Becaplermin | Regranex ™; | Anti-Ulcer Agents; | For topical treatment of | 123969 |
| | Regranex ™ (OMJ | Topical | skin ulcers (from | |
| | Pharmaceuticals) | | diabetes) | |
| Bevacizumab | Avastin ™; Avastin ™ | Antiangiogenesis | For treatment of | 27043 |
| | (Genentech Inc) | Agents; | metastatic colorectal | |
| | | Antineoplastic | cancer | |
| | | Agents | | |
| Bivalirudin | Angiomax ™; | Anticoagulants; | For treatment of | 70037 |
| | Angiomax ™ | Antithrombotic | heparin-induced | |
| | (Medicines Co or | Agents | thrombocytopenia | |
| | MDCO); Angiox ™ | | | |
| Bortezomib | | Proteosome | | |
| | | Inhibitors | | |
| Bosutinib | | Tyrosine Kinase | | 530 |
| | | Inhibitors | | |
| Botulinum Toxin | BOTOX ™ (Allegran | Anti-Wrinkle | For the treatment of | 23315 |
| Type A | Inc); BOTOX | Agents; | cervical dystonia in | |
| | Cosmetic ™ | Antidystonic | adults to decrease the | |
| | (Allegran Inc); | Agents; | severity of abnormal | |
| | Botox ™; Dysport ™ | Neuromuscular | head position and neck | |
| | | Blocking Agents | pain associated with | |
| | | | cervical dystonia. Also | |
| | | | for the treatment of | |
| | | | severe primary axillary | |
| | | | hyperhidrosis that is | |
| | | | inadequately managed | |
| | | | with topical | |
| Botulinum Toxin | Myobloc ™ (Solstice | Antidystonic Agents | For the treatment of | 12902 |
| Type B | Neurosciences); | | patients with cervical | |
| | Neurobloc ™ | | dystonia to reduce the | |
| | (Solstice | | severity of abnormal | |
| | Neurosciences) | | head position and neck | |
| | | | pain associated with | |
| | | | cervical dystonia. | |
| C5 inhibitor | (Jerini Ophthalmic); | Inhibitors of C5 | AMD | |
| | (Ophthotech) | | | |
| Cal101 | Calistoga | Pl3Kdelta Inhibitor | AMD, DME | |
| Canstatin | | | | |
| Capromab | ProstaScint ™ | Imaging Agents | For diagnosis of | 84331 |
| | (Cytogen Corp) | | prostate cancer and | |
| | | | detection of intra-pelvic | |
| | | | metastases | |
| Captopril | | ACE Inhibitors | | |
| CCI-779 | (Wyeth) | Limus Immunophilin | | |
| | | Binding | | |
| | | Compounds | | |
| Cediranib | | Tyrosine Kinase | | 450 |
| | | Inhibitors | | |
| Celecoxib | | Cyclooxygenase | | |
| | | Inhibitors | | |
| Cetrorelix | Cetrotide ™ | Hormone | For the inhibition of | 78617 |
| | | Antagonists; | premature LH surges | |
| | | Infertility Agents | in women undergoing | |
| | | | controlled ovarian | |
| | | | stimulation | |
| Cetuximab | Erbitux ™; Erbitux ™ | Antineoplastic | For treatment of | 42632 |
| | (ImClone Systems | Agents | metastatic colorectal | |
| | Inc) | | cancer. | |
| Choriogonadotropin | Novarel ™; | Fertility Agents; | For the treatment of | 78617 |
| alfa | Ovidrel ™. | Gonadotropins | female infertility | |
| | Pregnyl ™; Profasi ™ | | | |
| Cilary neurotrophic | (Neurotech) | Cilary neurotrophic | AMD | |
| factor | | factor | | |
| Coagulation Factor | Benefix ™ (Genetics | Coagulants; | For treatment of | 267012 |
| IX | Institute) | Thrombotic Agents | hemophilia (Christmas | |
| | | | disease). | |
| Coagulation factor | NovoSeven ™ (Novo | Coagulants; | For treatment of | 54732 |
| VIIa | Nordisk) | Thrombotic Agents | hemorrhagic | |
| | | | complications in | |
| | | | hemophilia A and B | |
| Colchicines | | | | |
| Collagenase | Cordase ™; Santyl ™ | Anti-Ulcer Agents; | For treatment of | 138885 |
| | (Advance | Topical | chronic dermal ulcers | |
| | Biofactures Corp); | | and severe skin burns | |
| | Xiaflextm ™ | | | |
| Complement factor | (Optherion); | Complement factor | AMD, Geographic | |
| H recombinant | (Taligen | H recombinant | Atrophy | |
| | Therapeutics) | | | |
| Compstatin | (Potentia | Complement Factor | AMD | |
| derivative peptide, | Pharmaceuticals) | C3 Inhibitors; | | |
| POT-4 | | Compstatin | | |
| | | Derivative Peptides | | |
| Corticotropin | ACTH ™; | Diagnostic Agents | For use as a diagnostic | 33927 |
| | Acethropan ™; | | agent in the screening | |
| | Acortan ™; Acthar ™; | | of patients presumed | |
| | Exacthin ™; H.P. | | to have adrenocortical | |
| | Acthar Gel ™; | | insufficiency. | |
| | Isactid ™; Purified | | | |
| | cortrophin gel ™; | | | |
| | Reacthin ™; | | | |
| | Solacthyl ™; Tubex | | | |
| Cosyntropin | Cortrosyn ™; | Diagnostic Agents | For use as a diagnostic | 33927 |
| | Synacthen depot ™ | | agent in the screening | |
| | | | of patients presumed | |
| | | | to have adrenocortical | |
| | | | insufficiency. | |
| Cyclophilins | | Limus Immunophilin | | |
| | | Binding | | |
| | | Compounds | | |
| Cyclosporine | Gengraf ™ (Abbott | Antifungal Agents; | For treatment of | 32953 |
| | labs); Neoral ™ | Antirheumatic | transplant rejection, | |
| | (Novartis); | Agents; | rheumatoid arthritis, | |
| | Restasis ™; | Dermatologic | severe psoriasis | |
| | Restasis ™ (Allergan | Agents; Enzyme | | |
| | Inc); Sandimmune ™ | Inhibitors; | | |
| | (Novartis); | Immunomodulatory | | |
| | Sangcya ™ | Agents; | | |
| | | Immunosuppressive | | |
| | | Agents | | |
| Daclizumab | Zenapax ™ | Immunomodulatory | For prevention of renal | 61118 |
| | (Hoffmann-La | Agents; | transplant rejection; | |
| | Roche Inc) | Immunosuppressive | Uveitis | |
| | | Agents | | |
| Darbepoetin alfa | Aranesp ™ (Amgen | Antianemic Agents | For the treatment of | 55066 |
| | Inc.) | | anemia (from renal | |
| | | | transplants or certain | |
| | | | HIV treatment) | |
| Dasatinib | | Tyrosine Kinase | | 488 |
| | | Inhibitors | | |
| Defibrotide | Dasovas ™; | Antithrombotic | Defibrotide is used to | 36512 |
| | Noravid ™; | Agents | treat or prevent a | |
| | Prociclide ™ | | failure of normal blood | |
| | | | flow (occlusive venous | |
| | | | disease, OVD) in the | |
| | | | liver of patients who | |
| | | | have had bone marrow | |
| | | | transplants or received | |
| | | | certain drugs such as | |
| | | | oral estrogens, | |
| | | | mercaptopurine, and | |
| | | | many others. | |
| Denileukin diftitox | Ontak ™ | Antineoplastic | For treatment of | 61118 |
| | | Agents | cutaneous T-cell | |
| | | | lymphoma | |
| Desmopressin | Adiuretin ™; | Antidiuretic Agents; | For the management | 46800 |
| | Concentraid ™; | Hemostatics; Renal | of primary nocturnal | |
| | Stimate ™ | Agents | enuresis and indicated | |
| | | | as antidiuretic | |
| | | | replacement therapy in | |
| | | | the management of | |
| | | | central diabetes | |
| | | | insipidus and for the | |
| | | | management of the | |
| | | | temporary polyuria and | |
| | | | polydipsia following | |
| | | | head trauma or | |
| | | | surgery in the pitu | |
| Dexamethasone | Ozurdex ™ | Glucocorticoid | DME, inflammation, | 392 |
| | (Allergan) | | macular edema | |
| | | | following branch retinal | |
| | | | vein occlusion (BRVO) | |
| | | | or central retinal vein | |
| | | | occlusion (CRVO) | |
| Diclofenac | | Cyclooxygenase | | |
| | | Inhibitors | | |
| Dithiocarbamate | | NFκB Inhibitor | | |
| Dornase Alfa | Dilor ™; Dilor-400 ™; | Enzyme | For the treatment of | 7656 |
| | Lufyllin ™; Lufyllin- | Replacement | cystic fibrosis. | (double |
| | 400 ™; | Agents | | strand) |
| | Neothylline ™; | | | |
| | Pulmozyme ™ | | | |
| | (Genentech Inc) | | | |
| Drotrecogin alfa | Xigris ™; Xigris ™ (Eli | Antisepsis Agents | For treatment of | 267012 |
| | Lilly & Co) | | severe sepsis | |
| Eculizumab | Soliris ™; Soliris ™ | Complement | AMD | 188333 |
| | (Alexion | Cascade Inhibitor | | |
| | Pharmaceuticals) | (Factor C5) | | |
| Efalizumab | Raptiva ™; | Immunomodulatory | For the treatment of | 128771 |
| | Raptiva ™ | Agents; | adult patients with | |
| | (Genentech Inc) | Immunosuppressive | moderate to severe | |
| | | Agents | chronic plaque | |
| | | | psoriasis, who are | |
| | | | candidates for | |
| | | | phototherapy or | |
| | | | systemic therapy. | |
| Endostatin | | | | |
| Enfuvirtide | Fuzeon ™; Fuzeon ™ | Anti-HIV Agents; | For treatment of HIV | 16768 |
| | (Roche | HIV Fusion | AIDS | |
| | Pharmaceuticals) | Inhibitors | | |
| Epoetin alfa | Epogen ™ (Amgen | Antianemic Agents | For treatment of | 55066 |
| | Inc.); Epogin ™ | | anemia (from renal | |
| | (Chugai); Epomax ™ | | transplants or certain | |
| | (Elanex); Eprex ™ | | HIV treatment) | |
| | (Janssen-Cilag. | | | |
| | Ortho Biologics | | | |
| | LLC); | | | |
| | NeoRecormon ™ | | | |
| | (Roche); Procrit ™ | | | |
| | (Ortho Biotech); | | | |
| | Recormon ™ | | | |
| | (Roche) | | | |
| Eptifibatide | Integrilin ™; | Anticoagulants; | For treatment of | 7128 |
| | Integrilin ™ | Antiplatelet Agents; | myocardial infarction | |
| | (Millennium Pharm) | Platelet | and acute coronary | |
| | | Aggregation | syndrome. | |
| | | Inhibitors | | |
| Erlotinib | | Tyrosine Kinase | | 393 |
| | | Inhibitors | | |
| Etanercept | Enbrel ™; Enbrel ™ | Antirheumatic | Uveitis, AMD | 25645 |
| | (Immunex Corp) | Agents; | | |
| | | Immunomodulatory | | |
| | | Agents | | |
| Everolimus | Novartis | Limus Immunophilin | AMD | |
| | | Binding | | |
| | | Compounds, mTOR | | |
| Exenatide | Byetta ™; Byetta ™ | | Indicated as adjunctive | 53060 |
| | (Amylin/Eli Lilly) | | therapy to improve | |
| | | | glycemic control in | |
| | | | patients with Type 2 | |
| | | | diabetes mellitus who | |
| | | | are taking metformin, a | |
| | | | sulfonylurea, or a | |
| | | | combination of both, | |
| | | | but have not achieved | |
| | | | adequate glycemic | |
| | | | control. | |
| FCFD4514S | Genentech/Roche | Complement | AMD, Geographic | |
| | | Cascade Inhibitor | Atrophy | |
| | | (Factor D) | | |
| Felypressin | Felipresina ™ [INN- | Renal Agents; | For use as an | 46800 |
| | Spanish]; | Vasoconstrictor | alternative to | |
| | Felipressina ™ | Agents | adrenaline as a | |
| | [DCIT]; | | 90ocalizing agent, | |
| | Felypressin ™ | | provided that local | |
| | [USAN:BAN:INN]; | | ischaemia is not | |
| | Felypressine ™ | | essential. | |
| | [INN-French]; | | | |
| | Felypressinum ™ | | | |
| | [INN-Latin]; | | | |
| | Octapressin ™ | | | |
| Fenretinide | Sirion/reVision | Binding Protein | AMD, Geographic | |
| | Therapeutics | Antagonist for Oral | Atrophy | |
| | | Vitamin A | | |
| Filgrastim | Neupogen ™ | Anti-Infective | Increases leukocyte | 28518 |
| | (Amgen Inc.) | Agents; | production, for | |
| | | Antineutropenic | treatment in non- | |
| | | Agents; | myeloid | |
| | | Immunomodulatory | cancer, neutropenia | |
| | | Agents | and bone marrow | |
| | | | transplant | |
| FK605-binding | | Limus Immunophilin | | |
| proteins, FKBPs | | Binding | | |
| | | Compounds | | |
| Fluocinolone | Retisert ™ (Bausch | Glucocorticoid | Retinal inflammation, | 453 |
| Acetonide | & Lomb); Iluvien ™ | | diabetic macular | |
| | (Alimera Sciences, | | edema | |
| | Inc.) | | | |
| Follitropin beta | Follistim ™ | Fertility Agents | For treatment of | 78296 |
| | (Organon); Gonal | | female infertility | |
| | F ™; Gonal-F ™ | | | |
| Fumagillin | | | | |
| Galsulfase | Naglazyme ™; | Enzyme | For the treatment of | 47047 |
| | Naglazyme ™ | Replacement | adults and children | |
| | (BioMarin | Agents | with | |
| | Pharmaceuticals) | | Mucopolysaccharidosis | |
| | | | VI. | |
| Gefitinib | | Tyrosine Kinase | | 447 |
| | | Inhibitors | | |
| Gemtuzumab | Mylotarg ™; | Antineoplastic | For treatment of acute | 39826 |
| ozogamicin | Mylotarg ™ (Wyeth) | Agents | myeloid leukemia | |
| Glatiramer Acetate | Copaxone ™ | Adjuvants, | For reduction of the | 29914 |
| | | Immunologic; | frequency of relapses | |
| | | Immunosuppressive | in patients with | |
| | | Agents | Relapsing-Remitting | |
| | | | Multiple Sclerosis. | |
| Glucagon | GlucaGen ™ (Novo | Antihypoglycemic | For treatment of | 54009 |
| recombinant | Nordisk); | Agents | severe hypoglycemia, | |
| | Glucagon ™ (Eli | | also used in | |
| | Lilly) | | gastrointestinal | |
| | | | imaging | |
| Goserelin | Zoladex ™ | Antineoplastic | Breast cancer; | 78617 |
| | | Agents; | Prostate carcinoma; | |
| | | Antineoplastic | Endometriosis | |
| | | Agents, Hormonal | | |
| Human Serum | Albutein ™ (Alpha | Serum substitutes | For treatment of | 39000 |
| Albumin | Therapeutic Corp) | | severe blood loss, | |
| | | | hypervolemia, | |
| | | | hypoproteinemia | |
| Hyaluronidase | Vitragan ™; | Anesthetic | For increase of | 69367 |
| | Vitrase ™; Vitrase ™ | Adjuvants; | absorption and | |
| | (Ista Pharma) | Permeabilizing | distribution of other | |
| | | Agents | injected drugs and for | |
| | | | rehydration | |
| Ibritumomab | Zevalin ™ (IDEC | Antineoplastic | For treatment of non- | 33078 |
| | Pharmaceuticals) | Agents | Hodgkin's lymphoma | |
| Idursulfase | Elaprase ™ (Shire | Enzyme | For the treatment of | 47047 |
| | Pharmaceuticals) | Replacement | Hunter syndrome in | |
| | | Agents | adults and children | |
| | | | ages 5 and older. | |
| Imatinib | | Tyrosine Kinase | AMD, DME | 494 |
| | | Inhibitors | | |
| Immune globulin | Civacir ™; | Anti-Infectives; | For treatment of | 42632 |
| | Flebogamma ™ | Immunomodulatory | immunodeficiencies, | |
| | (Instituto Grifols | Agents | thrombocytopenic | |
| | SA); Gamunex ™ | | purpura, Kawasaki | |
| | (Talecris | | disease, | |
| | Biotherapeutics) | | gammablobulinemia, | |
| | | | leukemia, bone | |
| | | | transplant | |
| Infliximab | Remicade ™ | Immunomodulatory | Uveitis, AMD | 25645 |
| | (Centocor Inc) | Agents; | | |
| | | Immunosuppressive | | |
| | | Agents | | |
| Insulin Glargine | Lantus ™ | Hypoglycemic | For treatment of | 156308 |
| recombinant | | Agents | diabetes (type I and II) | |
| Insulin Lyspro | Humalog ™ (Eli Lily); | Hypoglycemic | For treatment of | 154795 |
| recombinant | Insulin Lispro (Eli | Agents | diabetes (type I and II) | |
| | Lily) | | | |
| Insulin recombinant | Novolin R ™ (Novo | Hypoglycemic | For treatment of | 156308 |
| | Nordisk) | Agents | diabetes (type I and II) | |
| Insulin, porcine | Iletin II ™ | Hypoglycemic | For the treatment of | 156308 |
| | | Agents | diabetes (type I and II) | |
| Interferon | | | | |
| Interferon Alfa-2a, | Roferon A ™ | Antineoplastic | For treatment of | 57759 |
| Recombinant | (Hoffmann-La | Agents; Antiviral | chronic hepatitis C, | |
| | Roche Inc); | Agents | hairy cell leukemia, | |
| | Veldona ™ (Amarillo | | AIDS-related Kaposi's | |
| | Biosciences) | | sarcoma, and chronic | |
| | | | myelogenous | |
| | | | leukemia. Also for the | |
| | | | treatment of oral warts | |
| | | | arising from HIV | |
| | | | infection. | |
| Interferon Alfa-2b, | Intron A ™ (Schering | Antineoplastic | For the treatment of | 57759 |
| Recombinant | Corp) | Agents; Antiviral | hairy cell leukemia, | |
| | | Agents; | malignant melanoma, | |
| | | Immunomodulatory | and AIDS-related | |
| | | Agents | Kaposi's sarcoma. | |
| Interferon alfacon-1 | Advaferon ™; | Antineoplastic | For treatment of hairy | 57759 |
| | Infergen ™ | Agents; Antiviral | cell leukemia, | |
| | (InterMune Inc) | Agents; | malignant melanoma, | |
| | | Immunomodulatory | and AIDS-related | |
| | | Agents | Kaposi's sarcoma | |
| Interferon alfa-n1 | Wellferon ™ | Antiviral Agents; | For treatment of | 57759 |
| | (GlaxoSmithKline) | Immunomodulatory | venereal or genital | |
| | | Agents | warts caused by the | |
| | | | Human Papiloma Virus | |
| Interferon alfa-n3 | Alferon ™ (Interferon | Antineoplastic | For the intralesional | 57759 |
| | Sciences Inc.); | Agents; Antiviral | treatment of refractory | |
| | Alferon LDO ™; | Agents; | or recurring external | |
| | Alferon N Injection ™ | Immunomodulatory | condylomata | |
| | | Agents | 93cuminate. | |
| Interferon beta-1b | Betaseron ™ (Chiron | Antiviral Agents; | For treatment of | 57759 |
| | Corp) | Immunomodulatory | relapsing/remitting | |
| | | Agents | multiple sclerosis | |
| Interferon gamma- | Actimmune ™; | Antiviral Agents; | For treatment of | 37835 |
| 1b | Actimmune ™ | Immunomodulatory | Chronic granulomatous | |
| | (InterMune Inc) | Agents | disease, Osteopetrosis | |
| Lapatinib | | Tyrosine Kinase | | 581 |
| | | Inhibitors | | |
| Lepirudin | Refludan ™ | Anticoagulants; | For the treatment of | 70037 |
| | | Antithrombotic | heparin-induced | |
| | | Agents; Fibrinolytic | thrombocytopenia | |
| | | Agents | | |
| Lestaurtinib | | Tyrosine Kinase | | 439 |
| | | Inhibitors | | |
| Leuprolide | Eligard ™ (Atrix | Anti-Estrogen | For treatment of | 37731 |
| | Labs/QLT Inc) | Agents; | prostate cancer, | |
| | | Antineoplastic | endometriosis, uterine | |
| | | Agents | fibroids and premature | |
| | | | puberty | |
| Lutropin alfa | Luveris ™ (Serono) | Fertility Agents | For treatment of | 78617 |
| | | | female infertility | |
| Mecasermin | Increlex ™; | | For the long-term | 154795 |
| | Increlex ™ (Tercica); | | treatment of growth | |
| | Iplex | | failure in pediatric | |
| | | | patients with Primary | |
| | | | IGFD or with GH gene | |
| | | | deletion who have | |
| | | | developed neutralizing | |
| | | | antibodies to GH. It is | |
| | | | not indicated to treat | |
| | | | Secondary IGFD | |
| | | | resulting from GH | |
| | | | deficiency, | |
| | | | malnutrition, hypoth | |
| Menotropins | Repronex ™ | Fertility Agents | For treatment of | 78617 |
| | | | female infertility | |
| Methotrexate | | Immunomodulatory | Uveitis, DME | |
| mTOR inhibitors | | | | |
| Muromonab | Orthoclone OKT3 ™ | Immunomodulatory | For treatment of organ | 23148 |
| | (Ortho Biotech) | Agents; | transplant recipients, | |
| | | Immunosuppressive | prevention of organ | |
| | | Agents | rejection | |
| Natalizumab | Tysabri ™ | Immunomodulatory | For treatment of | 115334 |
| | | Agents | multiple sclerosis. | |
| Nepafenac | | Cyclooxygenase | | |
| | | Inhibitors | | |
| Nesiritide | Natrecor ™ | Cardiac drugs | For the intravenous | 118921 |
| | | | treatment of patients | |
| | | | with acutely | |
| | | | decompensated | |
| | | | congestive heart failure | |
| | | | who have dyspnea at | |
| | | | rest or with minimal | |
| | | | activity. | |
| Nilotinib | | Tyrosine Kinase | | 530 |
| | | Inhibitors | | |
| NS398 | | Cyclooxygenase | | |
| | | Inhibitors | | |
| Octreotide | Atrigel ™; | Anabolic Agents; | For treatment of | 42687 |
| | Longastatin ™; | Antineoplastic | acromegaly and | |
| | Sandostatin ™; | Agents, Hormonal; | reduction of side | |
| | Sandostatin LAR ™; | Gastrointestinal | effects from cancer | |
| | Sandostatin LAR ™ | Agents; Hormone | chemotherapy | |
| | (Novartis) | Replacement | | |
| | | Agents | | |
| Omalizumab | Xolair ™ (Genentech | Anti-Asthmatic | For treatment of | 29596 |
| | Inc) | Agents; | asthma caused by | |
| | | Immunomodulatory | allergies | |
| | | Agents | | |
| Oprelvekin | Neumega ™; | Coagulants; | Increases reduced | 45223 |
| | Neumega ™ | Thrombotics | platelet levels due to | |
| | (Genetics Institute | | chemotherapy | |
| | Inc) | | | |
| OspA lipoprotein | LYMErix ™ | Vaccines | For prophylactic | 95348 |
| | (SmithKline | | treatment of Lyme | |
| | Beecham) | | Disease | |
| OT-551 | (Othera) | Anti-oxidant | AMD | |
| | | eyedrop | | |
| Oxytocin | Oxytocin ™ (BAM | Anti-tocolytic | To assist in labor, | 12722 |
| | Biotech); Pitocin ™ | Agents; Labor | elective labor | |
| | (Parke-Davis); | Induction Agents; | induction, uterine | |
| | Syntocinon ™ | Oxytocics | contraction induction | |
| | (Sandoz) | | | |
| Palifermin | Kepivance ™ | Antimucositis | For treatment of | 138885 |
| | (Amgen Inc) | Agents | mucositis (mouth | |
| | | | sores) | |
| Palivizumab | Synagis ™ | Antiviral Agents | For treatment of | 63689 |
| | | | respiratory diseases | |
| | | | casued by respiratory | |
| | | | syncytial virus | |
| Panitumumab | Vectibix ™; | Antineoplastic | For the treatment of | 134279 |
| | Vectibix ™ (Amgen) | Agents | EGFR-expressing, | |
| | | | metastatic colorectal | |
| | | | carcinoma with | |
| | | | disease progression on | |
| | | | or following | |
| | | | fluoropyrimidine-, | |
| | | | oxaliplatin-, and | |
| | | | irinotecan-containing | |
| | | | chemotherapy | |
| | | | regimens. | |
| PDGF inhibitor | (Jerini Ophthalmic); | Inhibitors of PDGF | AMD | |
| | (Ophthotech) | | | |
| PEDF (pigment | | | | |
| epithelium derived | | | | |
| factor) | | | | |
| Pegademase | Adagen ™ (Enzon | Enzyme | For treatment of | 36512 |
| bovine | Inc.) | Replacement | adenosine deaminase | |
| | | Agents | deficiency | |
| Pegaptanib | Macugen ™ | Oligonucleotide | For the treatment of | 103121 |
| | | | neovascular (wet) age- | |
| | | | related macular | |
| | | | degeneration. | |
| Pegaspargase | Oncaspar ™ (Enzon | Antineoplastic | For treatment of acute | 132.118 |
| | Inc) | Agents | lymphoblastic leukemia | |
| Pegfilgrastim | Neulasta ™ (Amgen | Anti-Infective | Increases leukocyte | 28518 |
| | Inc.) | Agents; | production, for | |
| | | Antineutropenic | treatment in non- | |
| | | Agents; | myeloid cancer, | |
| | | Immunomodulatory | neutropenia and bone | |
| | | Agents | marrow transplant | |
| Peginterferon alfa- | Pegasys ™ | Antineoplastic | For treatment of hairy | 57759 |
| 2a | (Hoffman-La Roche | Agents; Antiviral | cell leukemia, | |
| | Inc) | Agents; | malignant melanoma, | |
| | | Immunomodulatory | and AIDS-related | |
| | | Agents | Kaposi's sarcoma. | |
| Peginterferon alfa- | PEG-Intron | Antineoplastic | For the treatment of | 57759 |
| 2b | (Schering Corp); | Agents; Antiviral | chronic hepatitis C in | |
| | Unitron PEG ™ | Agents; | patients not previously | |
| | | Immunomodulatory | treated with interferon | |
| | | Agents | alpha who have | |
| | | | compensated liver | |
| | | | disease and are at | |
| | | | least 18 years of age. | |
| Pegvisomant | Somavert ™ (Pfizer | Anabolic Agents; | For treatment of | 71500 |
| | Inc) | Hormone | acromegaly | |
| | | Replacement | | |
| | | Agents | | |
| Pentoxifylline | | | | |
| Perindozril | | ACE Inhibitors | | |
| Pimecrolimus | | Limus Immunophilin | | |
| | | Binding | | |
| | | Compounds | | |
| PKC (protein | | | | |
| kinase C) inhibitors | | | | |
| POT-4 | Potentia/Alcon | Complement | AMD | |
| | | Cascade Inhibitor | | |
| | | (Factor C3) | | |
| Pramlintide | Symlin ™; Symlin ™ | | For the mealtime | 16988 |
| | (Amylin | | treatment of Type I and | |
| | Pharmaceuticals) | | Type II diabetes in | |
| | | | combination with | |
| | | | standard insulin | |
| | | | therapy, in patients | |
| | | | who have failed to | |
| | | | achieve adequate | |
| | | | glucose control on | |
| | | | insulin monotherapy. | |
| Proteosome | Velcade ™ | | Proteosome inhibitors | |
| inhibitors | | | | |
| Pyrrolidine | | | | |
| Quinopril | | ACE Inhibitors | | |
| Ranibizumab | Lucentis ™ | | For the treatment of | 27043 |
| | | | patients with | |
| | | | neovascular (wet) age- | |
| | | | related macular | |
| | | | degeneration. | |
| Rapamycin | (MacuSight) | Limus Immunophilin | AMD | |
| (siroliums) | | Binding | | |
| | | Compounds | | |
| Rasburicase | Elitek ™; Elitek ™ | Antihyperuricemic | For treatment of | 168.11 |
| | (Sanofi-Synthelabo | Agents | hyperuricemia, | |
| | Inc); Fasturtec ™ | | reduces elevated | |
| | | | plasma uric acid levels | |
| | | | (from chemotherapy) | |
| Reteplase | Retavase ™ | Thrombolytic | For lysis of acute | 54732 |
| | (Centocor); | Agents | pulmonary emboli, | |
| | Retavase ™ (Roche) | | intracoronary emboli | |
| | | | and management of | |
| | | | myocardial infarction | |
| Retinal stimulant | Neurosolve ™ | Retinal stimulants | AMD | |
| | (Vitreoretinal | | | |
| | Technologies) | | | |
| Retinoid(s) | | | | |
| Rituximab | MabThera ™; | Antineoplastic | For treatment of B-cell | 33078 |
| | Rituxan ™ | Agents | non-Hodgkins | |
| | | | lymphoma (CD20 | |
| | | | positive) | |
| RNAI (RNA | | | | |
| interference of | | | | |
| angiogenic factors) | | | | |
| Rofecoxib | Vioxx ™; Ceoxx ™; | Cyclooxygenase | | |
| | Ceeoxx ™ (Merck & | Inhibitors | | |
| | Co.) | | | |
| Rosiglitazone | | Thiazolidinediones | | |
| Ruboxistaurin | Eli Lilly | Protein Kinase C | DME, diabetic | 469 |
| | | (PKC)-b Inhibitor | peripheral retinopathy | |
| Salmon Calcitonin | Calcimar ™; | Antihypocalcemic | For the treatment of | 57304 |
| | Miacalcin ™ | Agents; | post-menopausal | |
| | (Novartis) | Antiosteporotic | osteoporosis | |
| | | Agents; Bone | | |
| | | Density | | |
| | | Conservation | | |
| | | Agents | | |
| Sargramostim | Immunex ™; | Anti-Infective | For the treatment of | 46207 |
| | Leucomax ™ | Agents; | cancer and bone | |
| | (Novartis); | Antineoplastic | marrow transplant | |
| | Leukine ™; | Agents; | | |
| | Leukine ™ (Berlex | Immunomodulatory | | |
| | Laboratories Inc) | Agents | | |
| SAR 1118 | SARCode | Immunomodulatory | Dry eye, DME, | |
| | | Agent | conjunctivitis | |
| SDZ-RAD | | Limus Immunophilin | | |
| | | Binding | | |
| | | Compounds | | |
| Secretin | SecreFlo ™; | Diagnostic Agents | For diagnosis of | 50207 |
| | Secremax ™, | | pancreatic exocrine | |
| | SecreFlo ™ | | dysfunction and | |
| | (Repligen Corp) | | gastrinoma | |
| Selective inhibitor | | | | |
| of the factor 3 | | | | |
| complement | | | | |
| cascade | | | | |
| Selective inhibitor | | | | |
| of the factor 5 | | | | |
| complement | | | | |
| cascade | | | | |
| Semaxanib | | Tyrosine Kinase | | 238 |
| | | Inhibitors | | |
| Sermorelin | Geref ™ (Serono | Anabolic Agents; | For the treatment of | 47402 |
| | Pharma) | Hormone | dwarfism, prevention of | |
| | | Replacement | HIV-induced weight | |
| | | Agents | loss | |
| Serum albumin | Megatope ™ (IsoTex | Imaging Agents | For determination of | 39000 |
| iodinated | Diagnostics) | | total blood and plasma | |
| | | | volumes | |
| SF1126 | Semafore | Pl3k/mTOR | AMD, DME | |
| | | Inhibition | | |
| Sirolimus | (MacuSight) | Limus Immunophilin | AMD | |
| reformulation | | Binding | | |
| (rapamycin) | | Compounds | | |
| siRNA molecule | (Quark | siRNA molecule | AMD | |
| synthetic, FTP- | Pharmaceuticals) | synthetic | | |
| 801i-14 | | | | |
| Somatropin | BioTropin ™ (Biotech | Anabolic Agents; | For treatment of | 71500 |
| recombinant | General); | Hormone | dwarfism, acromegaly | |
| | Genotropin ™ | Replacement | and prevention of HIV- | |
| | (Pfizer); | Agents | induced weight loss | |
| | Humatrope ™ (Eli | | | |
| | Lilly); Norditropin ™ | | | |
| | (Novo Nordisk); | | | |
| | Nutropin ™ | | | |
| | (Genentech Inc.); | | | |
| | NutropinAQ ™ | | | |
| | (Genentech Inc.); | | | |
| | Protropin ™ | | | |
| | (Genentech Inc.); | | | |
| | Saizen ™ (Serono | | | |
| | SA); Serostim ™; | | | |
| | Serostim ™ (Serono | | | |
| | SA); Tev-Tropin ™ | | | |
| | (GATE) | | | |
| Squalamine | | | | |
| Streptokinase | Streptase ™ (Aventis | Thrombolytic | For the treatment of | 90569 |
| | Behringer GmbH) | Agents | acute evolving | |
| | | | transmural myocardial | |
| | | | infarction, pulmonary | |
| | | | embolism, deep vein | |
| | | | thrombosis, arterial | |
| | | | thrombosis or | |
| | | | embolism and | |
| | | | occlusion of | |
| | | | arteriovenous | |
| | | | cannulae | |
| Sunitinib | | Tyrosine Kinase | | 398 |
| | | Inhibitors | | |
| TA106 | Taligen | Complement | AMD | |
| | | Cascade Inhibitor | | |
| | | (Factor B) | | |
| Tacrolimus | | Limus Immunophilin | | |
| | | Binding | | |
| | | Compounds | | |
| Tenecteplase | TNKase ™ | Thrombolytic | For treatment of | 54732 |
| | (Genentech Inc) | Agents | myocardial infarction | |
| | | | and lysis of | |
| | | | intracoronary emboli | |
| Teriparatide | Apthela ™; | Bone Density | For the treatment of | 66361 |
| | Forsteo ™; Forteo ™; | Conservation | osteoporosis in men | |
| | Fortessa ™; | Agents | and postmenopausal | |
| | Opthia ™; Optia ™; | | women who are at high | |
| | Optiah ™; | | risk for having a | |
| | Zalectra ™; | | fracture. Also used to | |
| | Zelletra ™ | | increase bone mass in | |
| | | | men with primary or | |
| | | | hypogonadal | |
| | | | osteoporosis who are | |
| | | | at high risk for fracture. | |
| Tetrathiomolybdate | | | | |
| Thalidomide | Celgene | Anti-inflammatory, | Uveitis | |
| | | Anti-proliferative | | |
| Thyrotropin Alfa | Thyrogen ™ | Diagnostic Agents | For detection of | 86831 |
| | (Genzyme Inc) | | residueal or recurrent | |
| | | | thyroid cancer | |
| Tie-1 and Tie-2 | | | | |
| kinase inhibitors | | | | |
| Toceranib | | Tyrosine Kinase | | 396 |
| | | Inhibitors | | |
| Tositumomab | Bexxar ™ (Corixa | Antineoplastic | For treatment of non- | 33078 |
| | Corp) | Agents | Hodgkin's lymphoma | |
| | | | (CD20 positive, | |
| | | | follicular) | |
| TPN 470 analogue | | | | |
| Trastuzumab | Herceptin ™ | Antineoplastic | For treatment of | 137912 |
| | (Genentech) | Agents | HER2-positive | |
| | | | pulmonary breast | |
| | | | cancer | |
| Triamcinolone | Triesence ™ | Glucocorticoid | DME, For treatment of | 435 |
| acetonide | | | inflammation of the | |
| | | | retina | |
| Troglitazone | | Thiazolidinediones | | |
| Tumistatin | | | | |
| Urofollitropin | Fertinex ™ (Serono S.A.) | Fertility Agents | For treatment of | 78296 |
| | | | female infertility | |
| Urokinase | Abbokinase ™; | Thrombolytic | For the treatment of | 90569 |
| | Abbokinase ™ | Agents | 100ulmonary | |
| | (Abbott | | embolism, coronary | |
| | Laboratories) | | artery thrombosis and | |
| | | | IV catheter clearance | |
| Vandetanib | | Tyrosine Kinase | | 475 |
| | | Inhibitors | | |
| Vasopressin | Pitressin ™; | Antidiuretics; | For the treatment of | 46800 |
| | Pressyn ™ | Oxytocics; | enuresis, polyuria, | |
| | | Vasoconstrictor | diabetes insipidus, | |
| | | Agents | polydipsia and | |
| | | | oesophageal varices | |
| | | | with bleeding | |
| Vatalanib | | Tyrosine Kinase | | 347 |
| | | Inhibitors | | |
| VEGF receptor | | | | |
| kinase inhibitor | | | | |
| VEGF Trap | Aflibercept ™ | Genetically | DME, cancer, retinal | 96600 |
| | (Regneron | Engineered | vein occlusion, | |
| | Pharmaceuticals, | Antibodies | choroidal | |
| | Bayer HealthCare | | neovascularization, | |
| | AG) | | delay wound healing, | |
| | | | cancer treatment | |
| Visual Cycle | (Acucela) | Visual Cycle | AMD | |
| Modulator ACU- | | Modulator | | |
| 4229 | | | | |
| Vitamin(s) | | | | |
| Vitronectin receptor | | | | |
| antagonists | | | | |
| Volociximab | Ophthotech | alpha5beta1 | AMD | |
| | | Integrin Inhibitor | | |
| XL765 | Exelixis/Sanofi- | Pl3k/mTOR | AMD, DME | |
| | Aventis | Inhibition | | |
| 2-Methoxyestradiol | (Paloma | Angiogenesis | AMD | |
| analogs | Pharmaceuticals) | inhibitors | | |
| 3-aminothalidomide | | | | |
| 13-cis retinoic acid | Accutane TM | | | |
| | (Roche | | | |
| | Pharmaceuticals) | | | |
| A0003 | (Aqumen | A0003 | AMD | |
| | BioPharmaceuticals) | | | |
| A5b1 integrin | (Jerini Ophthalmic); | Inhibitors of a5b1 | AMD | |
| inhibitor | (Ophthotech) | integrin | | |
| Abarelix | Plenaxis ™ (Praecis | Anti-Testosterone | For palliative treatment | 37731 |
| | Pharmaceuticals) | Agents; | of advanced prostate | |
| | | Antineoplastic | cancer. | |
| | | Agents | | |
| Abatacept | Orencia ™ (Bristol- | Antirheumatic | For the second line | 37697 |
| | Myers Squibb) | Agents | reduction of the signs | |
| | | | and symptoms of | |
| | | | moderate-to-severe | |
| | | | active rheumatoid | |
| | | | arthritis, inducing | |
| | | | inducing major clinical | |
| | | | response, slowing the | |
| | | | progression of | |
| | | | structural damage, and | |
| | | | improving physical | |
| | | | function in adult | |
| | | | patients who have | |
| Abciximab | ReoPro ™; | Anticoagulants; | For treatment of | 42632 |
| | ReoPro ™ | Antiplatelet Agents | myocardial infarction, | |
| | (Centocor) | | adjunct to | |
| | | | percutaneous | |
| | | | 102oronary | |
| | | | intervention, unstable | |
| | | | angina | |
| ABT-578 | (Abbott | Limus Immunophilin | | |
| | Laboratories) | Binding | | |
| | | Compounds | | |
| Acetonide | | | | |
| Adalimumab | Humira ™ (Abbott | Antirheumatic | Uveitis, AMD | 25645 |
| | Laboratories) | Agents; | | |
| | | Immunomodulatory | | |
| | | Agents | | |
| Aldesleukin | Proleukin ™; | Antineoplastic | For treatment of adults | 61118 |
| | Proleukin ™ (Chiron | Agents | with metastatic renal | |
| | Corp) | | cell carcinoma | |
| Alefacept | Amevive ™ | Immunomodulatory | For treatment of | 42632 |
| | | Agents; | moderate to severe | |
| | | Immunosuppressive | chronic plaque | |
| | | Agents | psoriasis | |
| Alemtuzumab | Campath ™; | Antineoplastic | For treatment of B-cell | 6614 |
| | Campath ™ (ILEX | Agents | chronic lymphocytic | |
| | Pharmaceuticals | | leukemia | |
| | LP); MabCampath ™ | | | |
| Alpha-1-proteinase | Aralast ™ (Baxter); | Enzyme | For treatment of | 28518 |
| inhibitor | Prolastin ™ (Talecris | Replacement | panacinar emphysema | |
| | Biotherapeutics C | Agents | | |
| | formerly Bayer) | | | |
| Alteplase | Activase ™ | Thrombolytic | For management of | 54732 |
| | (Genentech Inc) | Agents | acute myocardial | |
| | | | infarction, acute | |
| | | | ischemic strok and for | |
| | | | lysis of acute | |
| | | | pulmonary emboli | |
| AMG-1470 | | | | |
| Anakinra | Kineret ™ (Amgen | Anti-Inflammatory | For the treatment of | 65403 |
| | Inc) | Agents, Non- | adult rheumatoid | |
| | | Steroidal; | arthritis. | |
| | | Antirheumatic | | |
| | | Agents; | | |
| | | Immunomodulatory | | |
| | | Agents | | |
| Anecortave acetate | | | | |
| Angiostatin | | | | |
| Anistreplase | Eminase ™ (Wulfing | Thrombolytic | For lysis of acute | 54732 |
| | Pharma GmbH) | Agents | pulmonary emboli, | |
| | | | intracoronary emboli | |
| | | | and management of | |
| | | | myocardial infarction | |
| Anti-angiogenesis | (Eyecopharm) | Anti-angiogenesis | AMD | |
| peptides | | peptides | | |
| Anti-angiogenesis | (TRACON Pharma) | Anti-angiogenesis | AMD | |
| antibodies, | | antibodies | | |
| TRC093, TRC105 | | | | |
| Anti-angiogeric | Icon-1 ™ (Iconic | Anti-angiogeric | AMD | |
| bifunctional protein | Therapeutics) | bifunctional protein, | | |
| | | Icon-1 | | |
| Anti-endothelial | | | | |
| growth factor | | | | |
| Antihemophilic | Advate ™; | Coagulants; | For the treatment of | 70037 |
| Factor | Alphanate ™; | Thrombotic Agents | hemophilia A, von | |
| | Bioclate ™; | | Willebrand diseae and | |
| | Helixate ™; Helixate | | Factor XIII deficiency | |
| | FS ™; Hemofil M ™; | | | |
| | Humate-P ™; | | | |
| | Hyate:C ™; Koate- | | | |
| | HP ™; Kogenate ™; | | | |
| | Kogenate FS ™; | | | |
| | Monarc-M ™; | | | |
| | Monoclate-P ™; | | | |
| | ReFacto ™; | | | |
| | Xyntha ™ | | | |
| Antithymocyte | Genzyme); | Immunomodulatory | For prevention of renal | 37173 |
| globulin | Thymoglobulin ™ | Agents | transplant rejection | |
| | (SangStat Medical | | | |
| Anti-hypertensive | (MacuCLEAR) | Anti-hypertensive | AMD | |
| MC1101 | | MC1101 | | |
| Anti-platelet | | | | |
| devired growth | | | | |
| factor | | | | |
| Anti-VEGF | (Neurotech); | Anti-VEGF | AMD | |
| | Avastin ™ | | | |
| | (NeoVista) | | | |
| AP23841 | (Ariad) | Limus Immunophilin | | |
| | | Binding | | |
| | | Compounds | | |
| ARC1905 | Ophthotech | Complement | | |
| | | Cascade Inhibitor | | |
| | | (Factor C5) | | |
| Aprotinin | Trasylol ™ | Antifibrinolytic | For prophylactic use to | 90569 |
| | | Agents | reduce perioperative | |
| | | | blood loss and the | |
| | | | need for blood | |
| | | | transfusion in patients | |
| | | | undergoing | |
| | | | cardiopulmonary | |
| | | | bypass in the course of | |
| | | | coronary artery bypass | |
| | | | graft surgery who are | |
| | | | at an increased risk for | |
| | | | blood loss and blood | |
| | | | transfusio | |
| Arcitumomab | CEA-Scan ™ | Diagnostic Agents; | For imaging colorectal | 57561 |
| | | Imaging Agents | tumors | |
| Asparaginase | Elspar ™ (Merck & | Antineoplastic | For treatment of acute | 132.118 |
| | Co. Inc) | Agents | lympocytic leukemia | |
| | | | and non-Hodgkins | |
| | | | lymphoma | |
| Axitinib | | Tyrosine Kinase | | 386 |
| | | Inhibitors | | |
| Basiliximab | Simulect ™ (Novartis | Immunomodulatory | For prophylactic | 61118 |
| | Pharmaceuticals) | Agents; | treatment of kidney | |
| | | Immunosuppressive | transplant rejection | |
| | | Agents | | |
| Becaplermin | Regranex ™; | Anti-Ulcer Agents; | For topical treatment of | 123969 |
| | Regranex ™ (OMJ | Topical | skin ulcers (from | |
| | Pharmaceuticals) | | diabetes) | |
| Bevacizumab | Avastin ™; Avastin ™ | Antiangiogenesis | For treatment of | 27043 |
| | (Genentech Inc) | Agents; | metastatic colorectal | |
| | | Antineoplastic | cancer | |
| | | Agents | | |
| Bivalirudin | Angiomax ™; | Anticoagulants; | For treatment of | 70037 |
| | Angiomax ™ | Antithrombotic | heparin-induced | |
| | (Medicines Co or | Agents | thrombocytopenia | |
| | MDCO); Angiox ™ | | | |
| Bortezomib | | Proteosome | | |
| | | Inhibitors | | |
| Bosutinib | | Tyrosine Kinase | | 530 |
| | | Inhibitors | | |
| Botulinum Toxin | BOTOX ™ (Allegran | Anti-Wrinkle | For the treatment of | 23315 |
| Type A | Inc); BOTOX | Agents; | cervical dystonia in | |
| | Cosmetic ™ | Antidystonic | adults to decrease the | |
| | (Allegran Inc); | Agents; | severity of abnormal | |
| | Botox ™; Dysport ™ | Neuromuscular | head position and neck | |
| | | Blocking Agents | pain associated with | |
| | | | cervical dystonia. Also | |
| | | | for the treatment of | |
| | | | severe primary axillary | |
| | | | hyperhidrosis that is | |
| | | | inadequately managed | |
| | | | with topical | |
| Botulinum Toxin | Myobloc ™ (Solstice | Antidystonic Agents | For the treatment of | 12902 |
| Type B | Neurosciences); | | patients with cervical | |
| | Neurobloc ™ | | dystonia to reduce the | |
| | (Solstice | | severity of abnormal | |
| | Neurosciences) | | head position and neck | |
| | | | pain associated with | |
| | | | cervical dystonia. | |
| C5 inhibitor | (Jerini Ophthalmic); | Inhibitors of C5 | AMD | |
| | (Ophthotech) | | | |
| Cal101 | Calistoga | Pl3Kdelta Inhibitor | AMD, DME | |
| Canstatin | | | | |
| Capromab | ProstaScint ™ | Imaging Agents | For diagnosis of | 84331 |
| | (Cytogen Corp) | | prostate cancer and | |
| | | | detection of intra-pelvic | |
| | | | metastases | |
| Captopril | | ACE Inhibitors | | |
| CCI-779 | (Wyeth) | Limus Immunophilin | | |
| | | Binding | | |
| | | Compounds | | |
| Cediranib | | Tyrosine Kinase | | 450 |
| | | Inhibitors | | |
| Celecoxib | | Cyclooxygenase | | |
| | | Inhibitors | | |
| Cetrorelix | Cetrotide ™ | Hormone | For the inhibition of | 78617 |
| | | Antagonists; | premature LH surges | |
| | | Infertility Agents | in women undergoing | |
| | | | controlled ovarian | |
| | | | stimulation | |
| Cetuximab | Erbitux ™; Erbitux ™ | Antineoplastic | For treatment of | 42632 |
| | (ImClone Systems | Agents | metastatic colorectal | |
| | Inc) | | cancer. | |
| Choriogonadotropin | Novarel ™; | Fertility Agents; | For the treatment of | 78617 |
| alfa | Ovidrel ™. | Gonadotropins | female infertility | |
| | Pregnyl ™; Profasi ™ | | | |
| Cilary neurotrophic | (Neurotech) | Cilary neurotrophic | AMD | |
| factor | | factor | | |
| Coagulation Factor | Benefix ™ (Genetics | Coagulants; | For treatment of | 267012 |
| IX | Institute) | Thrombotic Agents | hemophilia (Christmas | |
| | | | disease). | |
| Coagulation factor | NovoSeven ™ (Novo | Coagulants; | For treatment of | 54732 |
| VIIa | Nordisk) | Thrombotic Agents | hemorrhagic | |
| | | | complications in | |
| | | | hemophilia A and B | |
| Colchicines | | | | |
| Collagenase | Cordase ™; Santyl ™ | Anti-Ulcer Agents; | For treatment of | 138885 |
| | (Advance | Topical | chronic dermal ulcers | |
| | Biofactures Corp); | | and severe skin burns | |
| | Xiaflextm ™ | | | |
| Complement factor | (Optherion); | Complement factor | AMD, Geographic | |
| H recombinant | (Taligen | H recombinant | Atrophy | |
| | Therapeutics) | | | |
| Compstatin | (Potentia | Complement Factor | AMD | |
| derivative peptide, | Pharmaceuticals) | C3 Inhibitors; | | |
| POT-4 | | Compstatin | | |
| | | Derivative Peptides | | |
| Corticotropin | ACTH ™; | Diagnostic Agents | For use as a diagnostic | 33927 |
| | Acethropan ™; | | agent in the screening | |
| | Acortan ™; Acthar ™; | | of patients presumed | |
| | Exacthin ™; H.P. | | to have adrenocortical | |
| | Acthar Gel ™; | | insufficiency. | |
| | Isactid ™; Purified | | | |
| | cortrophin gel ™; | | | |
| | Reacthin ™; | | | |
| | Solacthyl ™; Tubex | | | |
| Cosyntropin | Cortrosyn ™; | Diagnostic Agents | For use as a diagnostic | 33927 |
| | Synacthen depot ™ | | agent in the screening | |
| | | | of patients presumed | |
| | | | to have adrenocortical | |
| | | | insufficiency. | |
| Cyclophilins | | Limus Immunophilin | | |
| | | Binding | | |
| | | Compounds | | |
| Cyclosporine | Gengraf ™ (Abbott | Antifungal Agents; | For treatment of | 32953 |
| | labs); Neoral ™ | Antirheumatic | transplant rejection, | |
| | (Novartis); | Agents; | rheumatoid arthritis, | |
| | Restasis ™; | Dermatologic | severe psoriasis | |
| | Restasis ™ (Allergan | Agents; Enzyme | | |
| | Inc); Sandimmune ™ | Inhibitors; | | |
| | (Novartis); | Immunomodulatory | | |
| | Sangcya ™ | Agents; | | |
| | | Immunosuppressive | | |
| | | Agents | | |
| Daclizumab | Zenapax ™ | Immunomodulatory | For prevention of renal | 61118 |
| | (Hoffmann-La | Agents; | transplant rejection; | |
| | Roche Inc) | Immunosuppressive | Uveitis | |
| | | Agents | | |
| Darbepoetin alfa | Aranesp ™ (Amgen | Antianemic Agents | For the treatment of | 55066 |
| | Inc.) | | anemia (from renal | |
| | | | transplants or certain | |
| | | | HIV treatment) | |
| Dasatinib | | Tyrosine Kinase | | 488 |
| | | Inhibitors | | |
| Defibrotide | Dasovas ™; | Antithrombotic | Defibrotide is used to | 36512 |
| | Noravid ™; | Agents | treat or prevent a | |
| | Prociclide ™ | | failure of normal blood | |
| | | | flow (occlusive venous | |
| | | | disease, OVD) in the | |
| | | | liver of patients who | |
| | | | have had bone marrow | |
| | | | transplants or received | |
| | | | certain drugs such as | |
| | | | oral estrogens, | |
| | | | mercaptopurine, and | |
| | | | many others. | |
| Denileukin diftitox | Ontak ™ | Antineoplastic | For treatment of | 61118 |
| | | Agents | cutaneous T-cell | |
| | | | lymphoma | |
| Desmopressin | Adiuretin ™; | Antidiuretic Agents; | For the management | 46800 |
| | Concentraid ™; | Hemostatics; Renal | of primary nocturnal | |
| | Stimate ™ | Agents | enuresis and indicated | |
| | | | as antidiuretic | |
| | | | replacement therapy in | |
| | | | the management of | |
| | | | central diabetes | |
| | | | insipidus and for the | |
| | | | management of the | |
| | | | temporary polyuria and | |
| | | | polydipsia following | |
| | | | head trauma or | |
| | | | surgery in the pitu | |
| Dexamethasone | Ozurdex ™ | Glucocorticoid | DME, inflammation, | 392 |
| | (Allergan) | | macular edema | |
| | | | following branch retinal | |
| | | | vein occlusion (BRVO) | |
| | | | or central retinal vein | |
| | | | occlusion (CRVO) | |
| Diclofenac | | Cyclooxygenase | | |
| | | Inhibitors | | |
| Dithiocarbamate | | NFκB Inhibitor | | |
| Dornase Alfa | Dilor ™; Dilor-400 ™; | Enzyme | For the treatment of | 7656 |
| | Lufyllin ™; Lufyllin- | Replacement | cystic fibrosis. | (double |
| | 400 ™; | Agents | | strand) |
| | Neothylline ™; | | | |
| | Pulmozyme ™ | | | |
| | (Genentech Inc) | | | |
| Drotrecogin alfa | Xigris ™; Xigris ™ (Eli | Antisepsis Agents | For treatment of | 267012 |
| | Lilly & Co) | | severe sepsis | |
| Eculizumab | Soliris ™; Soliris ™ | Complement | AMD | 188333 |
| | (Alexion | Cascade Inhibitor | | |
| | Pharmaceuticals) | (Factor C5) | | |
| Efalizumab | Raptiva ™; | Immunomodulatory | For the treatment of | 128771 |
| | Raptiva ™ | Agents; | adult patients with | |
| | (Genentech Inc) | Immunosuppressive | moderate to severe | |
| | | Agents | chronic plaque | |
| | | | psoriasis, who are | |
| | | | candidates for | |
| | | | phototherapy or | |
| | | | systemic therapy. | |
| Endostatin | | | | |
| Enfuvirtide | Fuzeon ™; Fuzeon ™ | Anti-HIV Agents; | For treatment of HIV | 16768 |
| | (Roche | HIV Fusion | AIDS | |
| | Pharmaceuticals) | Inhibitors | | |
| Epoetin alfa | Epogen ™ (Amgen | Antianemic Agents | For treatment of | 55066 |
| | Inc.); Epogin ™ | | anemia (from renal | |
| | (Chugai); Epomax ™ | | transplants or certain | |
| | (Elanex); Eprex ™ | | HIV treatment) | |
| | (Janssen-Cilag. | | | |
| | Ortho Biologics | | | |
| | LLC); | | | |
| | NeoRecormon ™ | | | |
| | (Roche); Procrit ™ | | | |
| | (Ortho Biotech); | | | |
| | Recormon ™ | | | |
| | (Roche) | | | |
| Eptifibatide | Integrilin ™; | Anticoagulants; | For treatment of | 7128 |
| | Integrilin ™ | Antiplatelet Agents; | myocardial infarction | |
| | (Millennium Pharm) | Platelet | and acute coronary | |
| | | Aggregation | syndrome. | |
| | | Inhibitors | | |
| Erlotinib | | Tyrosine Kinase | | 393 |
| | | Inhibitors | | |
| Etanercept | Enbrel ™; Enbrel ™ | Antirheumatic | Uveitis, AMD | 25645 |
| | (Immunex Corp) | Agents; | | |
| | | Immunomodulatory | | |
| | | Agents | | |
| Everolimus | Novartis | Limus Immunophilin | AMD | |
| | | Binding | | |
| | | Compounds, mTOR | | |
| Exenatide | Byetta ™; Byetta ™ | | Indicated as adjunctive | 53060 |
| | (Amylin/Eli Lilly) | | therapy to improve | |
| | | | glycemic control in | |
| | | | patients with Type 2 | |
| | | | diabetes mellitus who | |
| | | | are taking metformin, a | |
| | | | sulfonylurea, or a | |
| | | | combination of both, | |
| | | | but have not achieved | |
| | | | adequate glycemic | |
| | | | control. | |
| FCFD4514S | Genentech/Roche | Complement | AMD, Geographic | |
| | | Cascade Inhibitor | Atrophy | |
| | | (Factor D) | | |
| Felypressin | Felipresina ™ [INN- | Renal Agents; | For use as an | 46800 |
| | Spanish]; | Vasoconstrictor | alternative to | |
| | Felipressina ™ | Agents | adrenaline as a | |
| | [DCIT]; | | 109ocalizing agent, | |
| | Felypressin ™ | | provided that local | |
| | [USAN:BAN:INN]; | | ischaemia is not | |
| | Felypressine ™ | | essential. | |
| | [INN-French]; | | | |
| | Felypressinum ™ | | | |
| | [INN-Latin]; | | | |
| | Octapressin ™ | | | |
| Fenretinide | Sirion/reVision | Binding Protein | AMD, Geographic | |
| | Therapeutics | Antagonist for Oral | Atrophy | |
| | | Vitamin A | | |
| Filgrastim | Neupogen ™ | Anti-Infective | Increases leukocyte | 28518 |
| | (Amgen Inc.) | Agents; | production, for | |
| | | Antineutropenic | treatment in non- | |
| | | Agents; | myeloid | |
| | | Immunomodulatory | cancer, neutropenia | |
| | | Agents | and bone marrow | |
| | | | transplant | |
| FK605-binding | | Limus Immunophilin | | |
| proteins, FKBPs | | Binding | | |
| | | Compounds | | |
| Fluocinolone | Retisert ™ (Bausch | Glucocorticoid | Retinal inflammation, | 453 |
| Acetonide | & Lomb); Iluvien ™ | | diabetic macular | |
| | (Alimera Sciences, | | edema | |
| | Inc.) | | | |
| Follitropin beta | Follistim ™ | Fertility Agents | For treatment of | 78296 |
| | (Organon); Gonal | | female infertility | |
| | F ™; Gonal-F ™ | | | |
| Fumagillin | | | | |
| Galsulfase | Naglazyme ™; | Enzyme | For the treatment of | 47047 |
| | Naglazyme ™ | Replacement | adults and children | |
| | (BioMarin | Agents | with | |
| | Pharmaceuticals) | | Mucopolysaccharidosis | |
| | | | VI. | |
| Gefitinib | | Tyrosine Kinase | | 447 |
| | | Inhibitors | | |
| Gemtuzumab | Mylotarg ™; | Antineoplastic | For treatment of acute | 39826 |
| ozogamicin | Mylotarg ™ (Wyeth) | Agents | myeloid leukemia | |
| Glatiramer Acetate | Copaxone ™ | Adjuvants, | For reduction of the | 29914 |
| | | Immunologic; | frequency of relapses | |
| | | Immunosuppressive | in patients with | |
| | | Agents | Relapsing-Remitting | |
| | | | Multiple Sclerosis. | |
| Glucagon | GlucaGen ™ (Novo | Antihypoglycemic | For treatment of | 54009 |
| recombinant | Nordisk); | Agents | severe hypoglycemia, | |
| | Glucagon ™ (Eli | | also used in | |
| | Lilly) | | gastrointestinal | |
| | | | imaging | |
| Goserelin | Zoladex ™ | Antineoplastic | Breast cancer; | 78617 |
| | | Agents; | Prostate carcinoma; | |
| | | Antineoplastic | Endometriosis | |
| | | Agents, Hormonal | | |
| Human Serum | Albutein ™ (Alpha | Serum substitutes | For treatment of | 39000 |
| Albumin | Therapeutic Corp) | | severe blood loss, | |
| | | | hypervolemia, | |
| | | | hypoproteinemia | |
| Hyaluronidase | Vitragan ™; | Anesthetic | For increase of | 69367 |
| | Vitrase ™; Vitrase ™ | Adjuvants; | absorption and | |
| | (Ista Pharma) | Permeabilizing | distribution of other | |
| | | Agents | injected drugs and for | |
| | | | rehydration | |
| Ibritumomab | Zevalin ™ (IDEC | Antineoplastic | For treatment of non- | 33078 |
| | Pharmaceuticals) | Agents | Hodgkin's lymphoma | |
| Idursulfase | Elaprase ™ (Shire | Enzyme | For the treatment of | 47047 |
| | Pharmaceuticals) | Replacement | Hunter syndrome in | |
| | | Agents | adults and children | |
| | | | ages 5 and older. | |
| Imatinib | | Tyrosine Kinase | AMD, DME | 494 |
| | | Inhibitors | | |
| Immune globulin | Civacir ™; | Anti-Infectives; | For treatment of | 42632 |
| | Flebogamma ™ | Immunomodulatory | immunodeficiencies, | |
| | (Instituto Grifols | Agents | thrombocytopenic | |
| | SA); Gamunex ™ | | purpura, Kawasaki | |
| | (Talecris | | disease, | |
| | Biotherapeutics) | | gammablobulinemia, | |
| | | | leukemia, bone | |
| | | | transplant | |
| Infliximab | Remicade ™ | Immunomodulatory | Uveitis, AMD | 25645 |
| | (Centocor Inc) | Agents; | | |
| | | Immunosuppressive | | |
| | | Agents | | |
| Insulin Glargine | Lantus ™ | Hypoglycemic | For treatment of | 156308 |
| recombinant | | Agents | diabetes (type I and II) | |
| Insulin Lyspro | Humalog ™ (Eli Lily); | Hypoglycemic | For treatment of | 154795 |
| recombinant | Insulin Lispro (Eli | Agents | diabetes (type I and II) | |
| | Lily) | | | |
| Insulin recombinant | Novolin R ™ (Novo | Hypoglycemic | For treatment of | 156308 |
| | Nordisk) | Agents | diabetes (type I and II) | |
| Insulin, porcine | Iletin II ™ | Hypoglycemic | For the treatment of | 156308 |
| | | Agents | diabetes (type I and II) | |
| Interferon | | | | |
| Interferon Alfa-2a, | Roferon A ™ | Antineoplastic | For treatment of | 57759 |
| Recombinant | (Hoffmann-La | Agents; Antiviral | chronic hepatitis C, | |
| | Roche Inc); | Agents | hairy cell leukemia, | |
| | Veldona ™ (Amarillo | | AIDS-related Kaposi's | |
| | Biosciences) | | sarcoma, and chronic | |
| | | | myelogenous | |
| | | | leukemia. Also for the | |
| | | | treatment of oral warts | |
| | | | arising from HIV | |
| | | | infection. | |
| Interferon Alfa-2b, | Intron A ™ (Schering | Antineoplastic | For the treatment of | 57759 |
| Recombinant | Corp) | Agents; Antiviral | hairy cell leukemia, | |
| | | Agents; | malignant melanoma, | |
| | | Immunomodulatory | and AIDS-related | |
| | | Agents | Kaposi's sarcoma. | |
| Interferon alfacon-1 | Advaferon ™; | Antineoplastic | For treatment of hairy | 57759 |
| | Infergen ™ | Agents; Antiviral | cell leukemia, | |
| | (InterMune Inc) | Agents; | malignant melanoma, | |
| | | Immunomodulatory | and AIDS-related | |
| | | Agents | Kaposi's sarcoma | |
| Interferon alfa-n1 | Wellferon ™ | Antiviral Agents; | For treatment of | 57759 |
| | (GlaxoSmithKline) | Immunomodulatory | venereal or genital | |
| | | Agents | warts caused by the | |
| | | | Human Papiloma Virus | |
| Interferon alfa-n3 | Alferon ™ (Interferon | Antineoplastic | For the intralesional | 57759 |
| | Sciences Inc.); | Agents; Antiviral | treatment of refractory | |
| | Alferon LDO ™; | Agents; | or recurring external | |
| | Alferon N Injection ™ | Immunomodulatory | condylomata | |
| | | Agents | 112cuminate. | |
| Interferon beta-1b | Betaseron ™ (Chiron | Antiviral Agents; | For treatment of | 57759 |
| | Corp) | Immunomodulatory | relapsing/remitting | |
| | | Agents | multiple sclerosis | |
| Interferon gamma- | Actimmune ™; | Antiviral Agents; | For treatment of | 37835 |
| 1b | Actimmune ™ | Immunomodulatory | Chronic granulomatous | |
| | (InterMune Inc) | Agents | disease, Osteopetrosis | |
| Lapatinib | | Tyrosine Kinase | | 581 |
| | | Inhibitors | | |
| Lepirudin | Refludan ™ | Anticoagulants; | For the treatment of | 70037 |
| | | Antithrombotic | heparin-induced | |
| | | Agents; Fibrinolytic | thrombocytopenia | |
| | | Agents | | |
| Lestaurtinib | | Tyrosine Kinase | | 439 |
| | | Inhibitors | | |
| Leuprolide | Eligard ™ (Atrix | Anti-Estrogen | For treatment of | 37731 |
| | Labs/QLT Inc) | Agents; | prostate cancer, | |
| | | Antineoplastic | endometriosis, uterine | |
| | | Agents | fibroids and premature | |
| | | | puberty | |
| Lutropin alfa | Luveris ™ (Serono) | Fertility Agents | For treatment of | 78617 |
| | | | female infertility | |
| Mecasermin | Increlex ™; | | For the long-term | 154795 |
| | Increlex ™ (Tercica); | | treatment of growth | |
| | Iplex | | failure in pediatric | |
| | | | patients with Primary | |
| | | | IGFD or with GH gene | |
| | | | deletion who have | |
| | | | developed neutralizing | |
| | | | antibodies to GH. It is | |
| | | | not indicated to treat | |
| | | | Secondary IGFD | |
| | | | resulting from GH | |
| | | | deficiency, | |
| | | | malnutrition, hypoth | |
| Menotropins | Repronex ™ | Fertility Agents | For treatment of | 78617 |
| | | | female infertility | |
| Methotrexate | | Immunomodulatory | Uveitis, DME | |
| mTOR inhibitors | | | | |
| Muromonab | Orthoclone OKT3 ™ | Immunomodulatory | For treatment of organ | 23148 |
| | (Ortho Biotech) | Agents; | transplant recipients, | |
| | | Immunosuppressive | prevention of organ | |
| | | Agents | rejection | |
| Natalizumab | Tysabri ™ | Immunomodulatory | For treatment of | 115334 |
| | | Agents | multiple sclerosis. | |
| Nepafenac | | Cyclooxygenase | | |
| | | Inhibitors | | |
| Nesiritide | Natrecor ™ | Cardiac drugs | For the intravenous | 118921 |
| | | | treatment of patients | |
| | | | with acutely | |
| | | | decompensated | |
| | | | congestive heart failure | |
| | | | who have dyspnea at | |
| | | | rest or with minimal | |
| | | | activity. | |
| Nilotinib | | Tyrosine Kinase | | 530 |
| | | Inhibitors | | |
| NS398 | | Cyclooxygenase | | |
| | | Inhibitors | | |
| Octreotide | Atrigel ™; | Anabolic Agents; | For treatment of | 42687 |
| | Longastatin ™; | Antineoplastic | acromegaly and | |
| | Sandostatin ™; | Agents, Hormonal; | reduction of side | |
| | Sandostatin LAR ™; | Gastrointestinal | effects from cancer | |
| | Sandostatin LAR ™ | Agents; Hormone | chemotherapy | |
| | (Novartis) | Replacement | | |
| | | Agents | | |
| Omalizumab | Xolair ™ (Genentech | Anti-Asthmatic | For treatment of | 29596 |
| | Inc) | Agents; | asthma caused by | |
| | | Immunomodulatory | allergies | |
| | | Agents | | |
| Oprelvekin | Neumega ™; | Coagulants; | Increases reduced | 45223 |
| | Neumega ™ | Thrombotics | platelet levels due to | |
| | (Genetics Institute | | chemotherapy | |
| | Inc) | | | |
| OspA lipoprotein | LYMErix ™ | Vaccines | For prophylactic | 95348 |
| | (SmithKline | | treatment of Lyme | |
| | Beecham) | | Disease | |
| OT-551 | (Othera) | Anti-oxidant | AMD | |
| | | eyedrop | | |
| Oxytocin | Oxytocin ™ (BAM | Anti-tocolytic | To assist in labor, | 12722 |
| | Biotech); Pitocin ™ | Agents; Labor | elective labor | |
| | (Parke-Davis); | Induction Agents; | induction, uterine | |
| | Syntocinon ™ | Oxytocics | contraction induction | |
| | (Sandoz) | | | |
| Palifermin | Kepivance ™ | Antimucositis | For treatment of | 138885 |
| | (Amgen Inc) | Agents | mucositis (mouth | |
| | | | sores) | |
| Palivizumab | Synagis ™ | Antiviral Agents | For treatment of | 63689 |
| | | | respiratory diseases | |
| | | | casued by respiratory | |
| | | | syncytial virus | |
| Panitumumab | Vectibix ™; | Antineoplastic | For the treatment of | 134279 |
| | Vectibix ™ (Amgen) | Agents | EGFR-expressing, | |
| | | | metastatic colorectal | |
| | | | carcinoma with | |
| | | | disease progression on | |
| | | | or following | |
| | | | fluoropyrimidine-, | |
| | | | oxaliplatin-, and | |
| | | | irinotecan-containing | |
| | | | chemotherapy | |
| | | | regimens. | |
| PDGF inhibitor | (Jerini Ophthalmic); | Inhibitors of PDGF | AMD | |
| | (Ophthotech) | | | |
| PEDF (pigment | | | | |
| epithelium derived | | | | |
| factor) | | | | |
| Pegademase | Adagen ™ (Enzon | Enzyme | For treatment of | 36512 |
| bovine | Inc.) | Replacement | adenosine deaminase | |
| | | Agents | deficiency | |
| Pegaptanib | Macugen ™ | Oligonucleotide | For the treatment of | 103121 |
| | | | neovascular (wet) age- | |
| | | | related macular | |
| | | | degeneration. | |
| Pegaspargase | Oncaspar ™ (Enzon | Antineoplastic | For treatment of acute | 132.118 |
| | Inc) | Agents | lymphoblastic leukemia | |
| Pegfilgrastim | Neulasta ™ (Amgen | Anti-Infective | Increases leukocyte | 28518 |
| | Inc.) | Agents; | production, for | |
| | | Antineutropenic | treatment in non- | |
| | | Agents; | myeloid cancer, | |
| | | Immunomodulatory | neutropenia and bone | |
| | | Agents | marrow transplant | |
| Peginterferon alfa- | Pegasys ™ | Antineoplastic | For treatment of hairy | 57759 |
| 2a | (Hoffman-La Roche | Agents; Antiviral | cell leukemia, | |
| | Inc) | Agents; | malignant melanoma, | |
| | | Immunomodulatory | and AIDS-related | |
| | | Agents | Kaposi's sarcoma. | |
| Peginterferon alfa- | PEG-Intron | Antineoplastic | For the treatment of | 57759 |
| 2b | (Schering Corp); | Agents; Antiviral | chronic hepatitis C in | |
| | Unitron PEG ™ | Agents; | patients not previously | |
| | | Immunomodulatory | treated with interferon | |
| | | Agents | alpha who have | |
| | | | compensated liver | |
| | | | disease and are at | |
| | | | least 18 years of age. | |
| Pegvisomant | Somavert ™ (Pfizer | Anabolic Agents; | For treatment of | 71500 |
| | Inc) | Hormone | acromegaly | |
| | | Replacement | | |
| | | Agents | | |
| Pentoxifylline | | | | |
| Perindozril | | ACE Inhibitors | | |
| Pimecrolimus | | Limus Immunophilin | | |
| | | Binding | | |
| | | Compounds | | |
| PKC (protein | | | | |
| kinase C) inhibitors | | | | |
| POT-4 | Potentia/Alcon | Complement | AMD | |
| | | Cascade Inhibitor | | |
| | | (Factor C3) | | |
| Pramlintide | Symlin ™; Symlin ™ | | For the mealtime | 16988 |
| | (Amylin | | treatment of Type I and | |
| | Pharmaceuticals) | | Type II diabetes in | |
| | | | combination with | |
| | | | standard insulin | |
| | | | therapy, in patients | |
| | | | who have failed to | |
| | | | achieve adequate | |
| | | | glucose control on | |
| | | | insulin monotherapy. | |
| Proteosome | Velcade ™ | | Proteosome inhibitors | |
| inhibitors | | | | |
| Pyrrolidine | | | | |
| Quinopril | | ACE Inhibitors | | |
| Ranibizumab | Lucentis ™ | | For the treatment of | 27043 |
| | | | patients with | |
| | | | neovascular (wet) age- | |
| | | | related macular | |
| | | | degeneration. | |
| Rapamycin | (MacuSight) | Limus Immunophilin | AMD | |
| (siroliums) | | Binding | | |
| | | Compounds | | |
| Rasburicase | Elitek ™; Elitek ™ | Antihyperuricemic | For treatment of | 168.11 |
| | (Sanofi-Synthelabo | Agents | hyperuricemia, | |
| | Inc); Fasturtec ™ | | reduces elevated | |
| | | | plasma uric acid levels | |
| | | | (from chemotherapy) | |
| Reteplase | Retavase ™ | Thrombolytic | For lysis of acute | 54732 |
| | (Centocor); | Agents | pulmonary emboli, | |
| | Retavase ™ (Roche) | | intracoronary emboli | |
| | | | and management of | |
| | | | myocardial infarction | |
| Retinal stimulant | Neurosolve ™ | Retinal stimulants | AMD | |
| | (Vitreoretinal | | | |
| | Technologies) | | | |
| Retinoid(s) | | | | |
| Rituximab | MabThera ™; | Antineoplastic | For treatment of B-cell | 33078 |
| | Rituxan ™ | Agents | non-Hodgkins | |
| | | | lymphoma (CD20 | |
| | | | positive) | |
| RNAI (RNA | | | | |
| interference of | | | | |
| angiogenic factors) | | | | |
| Rofecoxib | Vioxx ™; Ceoxx ™; | Cyclooxygenase | | |
| | Ceeoxx ™ (Merck & | Inhibitors | | |
| | Co.) | | | |
| Rosiglitazone | | Thiazolidinediones | | |
| Ruboxistaurin | Eli Lilly | Protein Kinase C | DME, diabetic | 469 |
| | | (PKC)-b Inhibitor | peripheral retinopathy | |
| Salmon Calcitonin | Calcimar ™; | Antihypocalcemic | For the treatment of | 57304 |
| | Miacalcin ™ | Agents; | post-menopausal | |
| | (Novartis) | Antiosteporotic | osteoporosis | |
| | | Agents; Bone | | |
| | | Density | | |
| | | Conservation | | |
| | | Agents | | |
| Sargramostim | Immunex ™; | Anti-Infective | For the treatment of | 46207 |
| | Leucomax ™ | Agents; | cancer and bone | |
| | (Novartis); | Antineoplastic | marrow transplant | |
| | Leukine ™; | Agents; | | |
| | Leukine ™ (Berlex | Immunomodulatory | | |
| | Laboratories Inc) | Agents | | |
| SAR 1118 | SARCode | Immunomodulatory | Dry eye, DME, | |
| | | Agent | conjunctivitis | |
| SDZ-RAD | | Limus Immunophilin | | |
| | | Binding | | |
| | | Compounds | | |
| Secretin | SecreFlo ™; | Diagnostic Agents | For diagnosis of | 50207 |
| | Secremax ™, | | pancreatic exocrine | |
| | SecreFlo ™ | | dysfunction and | |
| | (Repligen Corp) | | gastrinoma | |
| Selective inhibitor | | | | |
| of the factor 3 | | | | |
| complement | | | | |
| cascade | | | | |
| Selective inhibitor | | | | |
| of the factor 5 | | | | |
| complement | | | | |
| cascade | | | | |
| Semaxanib | | Tyrosine Kinase | | 238 |
| | | Inhibitors | | |
| Sermorelin | Geref ™ (Serono | Anabolic Agents; | For the treatment of | 47402 |
| | Pharma) | Hormone | dwarfism, prevention of | |
| | | Replacement | HIV-induced weight | |
| | | Agents | loss | |
| Serum albumin | Megatope ™ (IsoTex | Imaging Agents | For determination of | 39000 |
| iodinated | Diagnostics) | | total blood and plasma | |
| | | | volumes | |
| SF1126 | Semafore | Pl3k/mTOR | AMD, DME | |
| | | Inhibition | | |
| Sirolimus | (MacuSight) | Limus Immunophilin | AMD | |
| reformulation | | Binding | | |
| (rapamycin) | | Compounds | | |
| siRNA molecule | (Quark | siRNA molecule | AMD | |
| synthetic, FTP- | Pharmaceuticals) | synthetic | | |
| 801i-14 | | | | |
| Somatropin | BioTropin ™ (Biotech | Anabolic Agents; | For treatment of | 71500 |
| recombinant | General); | Hormone | dwarfism, acromegaly | |
| | Genotropin ™ | Replacement | and prevention of HIV- | |
| | (Pfizer); | Agents | induced weight loss | |
| | Humatrope ™ (Eli | | | |
| | Lilly); Norditropin ™ | | | |
| | (Novo Nordisk); | | | |
| | Nutropin ™ | | | |
| | (Genentech Inc.); | | | |
| | NutropinAQ ™ | | | |
| | (Genentech Inc.); | | | |
| | Protropin ™ | | | |
| | (Genentech Inc.); | | | |
| | Saizen ™ (Serono | | | |
| | SA); Serostim ™; | | | |
| | Serostim ™ (Serono | | | |
| | SA); Tev-Tropin ™ | | | |
| | (GATE) | | | |
| Squalamine | | | | |
| Streptokinase | Streptase ™ (Aventis | Thrombolytic | For the treatment of | 90569 |
| | Behringer GmbH) | Agents | acute evolving | |
| | | | transmural myocardial | |
| | | | infarction, pulmonary | |
| | | | embolism, deep vein | |
| | | | thrombosis, arterial | |
| | | | thrombosis or | |
| | | | embolism and | |
| | | | occlusion of | |
| | | | arteriovenous | |
| | | | cannulae | |
| Sunitinib | | Tyrosine Kinase | | 398 |
| | | Inhibitors | | |
| TA106 | Taligen | Complement | AMD | |
| | | Cascade Inhibitor | | |
| | | (Factor B) | | |
| Tacrolimus | | Limus Immunophilin | | |
| | | Binding | | |
| | | Compounds | | |
| Tenecteplase | TNKase ™ | Thrombolytic | For treatment of | 54732 |
| | (Genentech Inc) | Agents | myocardial infarction | |
| | | | and lysis of | |
| | | | intracoronary emboli | |
| Teriparatide | Apthela ™; | Bone Density | For the treatment of | 66361 |
| | Forsteo ™; Forteo ™; | Conservation | osteoporosis in men | |
| | Fortessa ™; | Agents | and postmenopausal | |
| | Opthia ™; Optia ™; | | women who are at high | |
| | Optiah ™; | | risk for having a | |
| | Zalectra ™; | | fracture. Also used to | |
| | Zelletra ™ | | increase bone mass in | |
| | | | men with primary or | |
| | | | hypogonadal | |
| | | | osteoporosis who are | |
| | | | at high risk for fracture. | |
| Tetrathiomolybdate | | | | |
| Thalidomide | Celgene | Anti-inflammatory, | Uveitis | |
| | | Anti-proliferative | | |
| Thyrotropin Alfa | Thyrogen ™ | Diagnostic Agents | For detection of | 86831 |
| | (Genzyme Inc) | | residueal or recurrent | |
| | | | thyroid cancer | |
| Tie-1 and Tie-2 | | | | |
| kinase inhibitors | | | | |
| Toceranib | | Tyrosine Kinase | | 396 |
| | | Inhibitors | | |
| Tositumomab | Bexxar ™ (Corixa | Antineoplastic | For treatment of non- | 33078 |
| | Corp) | Agents | Hodgkin's lymphoma | |
| | | | (CD20 positive, | |
| | | | follicular) | |
| TPN 470 analogue | | | | |
| Trastuzumab | Herceptin ™ | Antineoplastic | For treatment of | 137912 |
| | (Genentech) | Agents | HER2-positive | |
| | | | pulmonary breast | |
| | | | cancer | |
| Triamcinolone | Triesence ™ | Glucocorticoid | DME, For treatment of | 435 |
| acetonide | | | inflammation of the | |
| | | | retina | |
| Troglitazone | | Thiazolidinediones | | |
| Tumistatin | | | | |
| Urofollitropin | Fertinex ™ (Serono | Fertility Agents | For treatment of | 78296 |
| | S.A.) | | female infertility | |
| Urokinase | Abbokinase ™; | Thrombolytic | For the treatment of | 90569 |
| | Abbokinase ™ | Agents | 120ulmonary | |
| | (Abbott | | embolism, coronary | |
| | Laboratories) | | artery thrombosis and | |
| | | | IV catheter clearance | |
| Vandetanib | | Tyrosine Kinase | | 475 |
| | | Inhibitors | | |
| Vasopressin | Pitressin ™; | Antidiuretics; | For the treatment of | 46800 |
| | Pressyn ™ | Oxytocics; | enuresis, polyuria, | |
| | | Vasoconstrictor | diabetes insipidus, | |
| | | Agents | polydipsia and | |
| | | | oesophageal varices | |
| | | | with bleeding | |
| Vatalanib | | Tyrosine Kinase | | 347 |
| | | Inhibitors | | |
| VEGF receptor | | | | |
| kinase inhibitor | | | | |
| VEGF Trap | Aflibercept ™ | Genetically | DME, cancer, retinal | 96600 |
| | (Regneron | Engineered | vein occlusion, | |
| | Pharmaceuticals, | Antibodies | choroidal | |
| | Bayer HealthCare | | neovascularization, | |
| | AG) | | delay wound healing, | |
| | | | cancer treatment | |
| Visual Cycle | (Acucela) | Visual Cycle | AMD | |
| Modulator ACU- | | Modulator | | |
| 4229 | | | | |
| Vitamin(s) | | | | |
| Vitronectin receptor | | | | |
| antagonists | | | | |
| Volociximab | Ophthotech | alpha5beta1 | AMD | |
| | | Integrin Inhibitor | | |
| XL765 | Exelixis/Sanofi- | Pl3k/mTOR | AMD, DME | |
| | Aventis | Inhibition | | |

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1.-64. (canceled)

65. An apparatus to determine a release rate of a therapeutic agent through a porous structure by measuring diffusion of one or more components of compressible fluids, the apparatus comprising:
a support to receive the porous structure having a first side and a second, opposite side, the porous structure affixed to an implantable therapeutic device;
a first source of a first fluid;
a second source of a second fluid;
a first chamber configured to contain the first fluid in fluid communication with the first side of the porous structure at a first pressure; and
a second chamber configured to be in fluid communication with the second side of the porous structure at a second pressure,
wherein the first pressure substantially equals the second pressure such that diffusion of one or more components of the first and second fluids is driven by a concentration gradient between the first chamber and the second chamber and pressure-driven convective gas flow across the porous structure is substantially inhibited; and
a detector in fluid communication with one or both of the first and second chambers, the detector configured to measure an amount of one or more components of the first fluid or the second fluid after the first chamber and the second chamber are placed in fluid communication through the porous structure for a period of time.

66. The apparatus of claim 65, wherein the therapeutic device further comprises a penetrable barrier disposed on a proximal end of the device, wherein the barrier is configured to be repeatedly pierced by a needle.

67. The apparatus of claim 66, wherein the first chamber of the apparatus is a reservoir chamber of the implantable therapeutic device and wherein the first fluid is injected into the reservoir chamber via a needle penetrating the barrier.

68. The apparatus of claim 67, wherein the detector is in fluid communication with the second chamber.

69. The apparatus of claim 68, wherein the amounts of the one or more components in the first chamber and the second chamber are unequal.

70. The apparatus of claim 69, wherein the amount of the one or more components in the second chamber is zero and the amount of the one or more components in the first chamber is greater than zero.

71. The apparatus of claim 70, wherein the detector measures the amount of the one or more components in the second chamber after the period of time of fluid communication between the first and second chambers and a concentration of the one or more components is calculated from the measured amounts and the volume of the reservoir chamber.

72. The apparatus of claim 71, wherein the period of time is at least about one tenth of one second and is equal to a length of time the fluid diffuses and accumulates.

73. The apparatus of claim 65, wherein the first fluid comprises one or more of, an elemental gas, helium gas, nitrogen gas, oxygen gas, a noble gas, neon gas, argon gas, krypton gas, xenon gas, a compound gas molecule comprising a plurality of elements, carbon dioxide, nitrous oxide, a mixture of gas, air, or water vapor.

74. The apparatus of claim 65, wherein the first fluid is helium and wherein the second fluid is nitrogen.

75. The apparatus of claim 74, wherein the detector measures helium.

76. The apparatus of claim 65, further comprising a valve positioned in the support to couple the first chamber to the second chamber through the porous structure when the valve is open.

77. The apparatus of claim 76, further comprising a pressure coupling device to inhibit flow of the first fluid and the second fluid through the porous structure.

78. The apparatus of claim 77, wherein the pressure coupling device is configured to couple the first pressure of the first chamber to the second pressure of the second chamber such that the first pressure substantially equals the second pressure.

79. The apparatus of claim 78, wherein the pressure coupling device comprises one or more of a diaphragm coupled between the first chamber or the second chamber, a pressure equalization column, or atmospheric pressure coupled to the first chamber and the second chamber.

80. A method of manufacturing a therapeutic device implantable in an eye for prolonged treatment of the eye, the method comprising:
performing a non-destructive test on a first porous structure, the non-destructive test relying on a gas concentration gradient and having zero pressure differential, wherein the first porous structure is configured to be coupled to a therapeutic device implantable for prolonged treatment;
obtaining from the non-destructive test at least one performance result for the first porous structure, wherein the at least one performance result comprises diffusional resistance measurement data;
measuring a diffusion rate of a drug according to passive, concentration-gradient driven molecular diffusion to obtain a measured diffusion rate, wherein the drug diffuses through a porous structure that is the same as the first porous structure or a different porous structure; and
correlating the at least one performance result of the first porous structure to the measured diffusion rate so as to form a correlation used to predict a measured diffusion rate of the drug through a second porous structure.

81. The method as in claim 80, further comprising:
performing the non-destructive test on at least a second porous structure;
obtaining from the non-destructive test at least one performance result for the at least a second porous structure; and
predicting, based on the correlation, a diffusion rate of the drug through the at least a second porous structure to obtain a predicted diffusion rate.

82. The method as in claim 81, further comprising identifying the at least a second porous structure as suitable for assembly with the therapeutic device.

83. The method as in claim 81, wherein the predicted diffusion rate corresponds to the diffusion rate of the drug through the at least one porous structure.

84. The method as in claim 81, further comprising measuring a diffusion rate of a drug through the second porous structure to obtain a second measured diffusion rate and comparing the second measured diffusion rate to the predicted diffusion rate.

85. The method as in claim 84, wherein measuring is performed prior to manufacturing the device with the second porous structure.

86. The method as in claim 84, wherein measuring is performed after manufacturing the device with the second porous structure.

87. The method as in claim 81, further comprising manufacturing the therapeutic device with the second porous structure without measuring a diffusion rate of a drug through the at least a second porous structure and comparing to the predicted diffusion rate.

88. The method as in claim 80, wherein the non-destructive test is performed on the first porous structure prior to assembling the at least one porous structure with the therapeutic device.

89. The method as in claim 80, wherein the non-destructive test is performed on the first porous structure after at least partially assembling the first porous structure with the therapeutic device.

90. The method as in claim 89, wherein the at least partially assembled therapeutic device includes a therapeutic agent contained in a reservoir.

91. A therapeutic device manufactured according to the method of claim 80.
